(19) United States
(12) Patent Application Publication
(10) Pub. No.: US 2025/0186087 A1
(43) Pub. Date: Jun. 12, 2025

(54) Bunion Correction Guide System and Methods

(71) Applicant: KI Orthopedics, Inc., Woodland Hills, CA (US)

(72) Inventors: Randall Allard, Golden, CO (US); Alireza Khosroabadi, Woodland Hills, CA (US); Todd Martens, Woodland Hills, CA (US); Thomas J. McLeer, Laguna Niguel, CA (US)

(21) Appl. No.: 18/974,699

(22) Filed: Dec. 9, 2024

Related U.S. Application Data
(60) Provisional application No. 63/608,255, filed on Dec. 9, 2023.
(60) Provisional application No. 63/659,124, filed on Jun. 12, 2024.

(51) Int. Cl. A61B 17/56; A61B 17/17

(52) U.S. Cl. CPC A61B 17/56; A61B 17/1775; A61B 2017/565

(57) ABSTRACT

The invention disclosed herein describes a bunion correction guide system or bunion guide system that is a percutaneous, minimally invasive system (MIS) to multi-directionally control or align the first metatarsal capital fragment (distal articular portion of the first metatarsal or metatarsal head) in four (4D) dimensions to its desired or proper anatomical position and cosmetic alignment by operating different mechanical actuation mechanisms.

### CROSS-REFERENCE TO RELATED APPLICATIONS

This application claims the benefit of U.S. Provisional Application No. 63/608,255 entitled “Bunion Correction Guide System and Methods” filed on Dec. 9, 2023, and U.S. Provisional Appl. No. 63/659,124, entitled “Bunion Correction Guide System and Methods” filed on Jun. 12, 2024, the disclosures of which are both incorporated by reference herein in their entireties.

### TECHNICAL FIELD

The invention relates to methods, devices, and systems to correct a bunion, or hallux valgus. More specifically, the invention relates to methods, devices and systems that will gradually correct and restore the toe deformity using multidirectional movement to achieve a normal anatomical positioning.

### BACKGROUND OF THE INVENTION

A bunion, or hallux valgus, is the tilting of the toe away from the mid-line of the foot. While bunions can theoretically occur in any toe, they typically occur in the big toe and only very occasionally in the little toe. Hallux valgus is typically characterized by a prominence made of bone that may be red, swollen and/or painful on the inside of the foot in and around the affected toe joint. Pain from a bunion can range from mild to severe, making it difficult to walk in different types of shoes.

Bunion treatments vary depending on the severity of pain and deformity related to the bunion. Bunion treatments may range from medications, wearable devices and appliances designed to correct bunions without surgery, and surgery. Medications, such as anti-inflammatory drugs or cortisone injections may be administered to ease pain and inflammation caused by joint deformities. However, such medications do not correct or reverse such ongoing joint deformities. Likewise, the wearable devices and appliances are designed to pull or push on one or more toes forcing the afflicted toe toward its proper orientation or positioning. While useful, the known devices tend to be bulky, uncomfortable and require consistent patient compliance. Probably the most extreme treatment for bunions is surgery. While pain and deformity are significantly reduced in the great majority of patients who undergo bunion surgery, bunion surgery in and its associated recovery can be lengthy, painful and costly.

### BRIEF DESCRIPTION OF THE SEVERAL VIEWS OF THE DRAWINGS

FIGS. 1A-1B depicts a top and isometric view of a first embodiment of a bunion correction guide system disposed over a foot;

FIGS. 2A-2B depicts a top and isometric view of a second embodiment of a bunion correction guide system disposed over a foot;

FIGS. 3A-3E depicts various views of a third embodiment of a bunion correction guide system disposed over a foot;

FIG. 4 depicts a isometric view of a fourth embodiment of a bunion correction guide system disposed over a foot;

FIG. 5 depicts an exploded isometric view of the bunion correction guide system of FIGS. 1A-1B;

FIG. 6 depicts an exploded isometric view of the bunion correction guide system of FIGS. 2A-2B;

FIG. 7 depicts an exploded isometric view of the bunion correction guide system of FIGS. 3A-3E;

FIG. 8 depicts an exploded isometric view of the bunion correction guide system of FIG. 4;

FIG. 9 depicts an exploded isometric view of the first actuation mechanism of the bunion correction guide system of FIGS. 1A-1B;

FIG. 10 depicts an exploded isometric view of the first actuation mechanism of the bunion correction guide system of FIGS. 2A-2B;

FIG. 11 depicts an exploded isometric view of the first actuation mechanism of the bunion correction guide system of FIGS. 3A-3E;

FIG. 12 depicts an exploded isometric view of the first actuation mechanism of the bunion correction guide system of FIG. 4;

FIGS. 13A-13F depicts different plan views of one embodiment of an angled base plate of the first actuation mechanism of FIG. 9;

FIGS. 14A-14H depicts different plan views of one embodiment of an angled base plate of the first actuation mechanism of FIG. 12;

FIGS. 15A-15G depicts different plan views of a tarsal base of the first actuation mechanism of FIG. 9;

FIGS. 15H-15I depicts a cross-sectional views of the tarsal base of the first actuation mechanism of FIG. 9;

FIGS. 16A-16F depicts different plan views of a tarsal base of the first actuation mechanism of FIG. 10;

FIG. 16G depicts a cross-sectional view of the tarsal base of FIGS. 16A-16F;

FIGS. 16H-16J depicts different plan views of a stabilization block of the tarsal base of FIGS. 16A-16F;

FIGS. 17A-17H depicts different plan views of a tarsal base of the first actuation mechanism of FIG. 12;

FIGS. 18A-18E depicts different plan views of a distraction screw of the first embodiment actuation mechanism of FIG. 9;

FIGS. 19A-19D depicts different plan views of a distraction screw and the distraction screw knob of the bunion guide system of FIG. 10;

FIGS. 20A-20E depicts different plan views of a distraction screw of the bunion guide system of FIG. 11;

FIGS. 21A-21F depicts different plan views of a distraction screw and ratchet wrench of the bunion guide system of FIG. 12;

FIGS. 22A-22E depicts different plan views of the ratchet wrench of FIGS. 21A-21F;

FIGS. 22F-22G depicts cross-sectional views of the ratchet wrench of FIGS. 22A-22E;

FIG. 22H depicts an isometric exploded view of the ratchet wrength of FIGS. 22A-22E;

FIGS. 23A-23E depict various views of an embodiment of a ratchet arm of the ratchet wrench of FIGS. 22A-22E;

FIGS. 24A-24F depict various views of an embodiment of a drive gear base of the ratchet wrench of FIG. 22A-22E;

FIG. 25 depicts isometric views a second actuation mechanism of the bunion correction guide system of FIGS. 1A-1B;

FIG. 26 depicts isometric views a second actuation mechanism of the bunion correction guide system of FIGS. 2A-2B;

FIG. 27 depicts isometric views a second actuation mechanism of the bunion correction guide system of FIG. 4;

FIGS. 28A-28H depicts different plan views of a rotation gear rack of the second actuation mechanism of FIG. 25;

FIGS. 29A-29F depicts different plan views of a rotation gear rack of the second actuation mechanism of FIG. 26;

FIGS. 30A-30F depicts different plan views of a rotation gear rack of the second actuation mechanism of FIG. 27;

FIGS. 31A-31E depicts different plan views of an arc gear knob of the second actuation mechanism of FIG. 25;

FIGS. 32A-32E depicts different plan views of an arc gear knob of the second actuation mechanism of FIG. 27;

FIGS. 33A-33H depicts different plan views of one embodiment of a rotation block of the second actuation mechanism of FIG. 25;

FIGS. 34A-34H depicts different plan views of one embodiment of a rotation block of the second actuation mechanism of FIG. 27;

FIGS. 34I-34J depicts different plan views of a rotation block of the second actuation mechanism of FIG. 27;

FIG. 35 depicts an isometric view of a third actuation mechanism of the bunion correction guide system of FIGS. 1A-1B;

FIG. 36 depicts an isometric view of a third actuation mechanism of the bunion correction guide system of FIGS. 2A-2B;

FIG. 37 depicts an isometric view of a third actuation mechanism of the bunion correction guide system of FIGS. 3A-3E;

FIG. 38 depicts an isometric view of a third actuation mechanism of the bunion correction guide system of FIG. 4;

FIGS. 39A-39G depicts different plan views of one embodiment of a displacement screw housing of the third actuation mechanism of FIG. 35;

FIGS. 40A-40H depicts different plan views of a displacement screw housing of the third actuation mechanism of FIG. 38;

FIG. 40I depicts a cross-section view of the displacement screw housing of FIGS. 40A-40H;

FIGS. 41A-41F depicts different plan views of a displacement screw of the third actuation mechanism of FIG. 35;

FIGS. 42A-42F depicts different plan views and a cross-section view of a displacement screw of the third actuation mechanism of FIG. 36;

FIGS. 43A-43E depicts different plan views of a displacement screw of the third actuation mechanism of FIG. 38;

FIGS. 44A-44H depicts different plan views of one embodiment of a top cap of the third actuation mechanism of FIG. 35;

FIG. 45 depicts an isometric view of a fourth actuation mechanism of the bunion correction guide system of FIGS. 1A-1B;

FIG. 46 depicts an isometric view of a fourth actuation mechanism of the bunion correction guide system of FIGS. 2A-2B;

FIG. 47 depicts an isometric view of a fourth actuation mechanism of the bunion correction guide system of FIGS. 3A-3E;

FIGS. 48A-48D depict various plan views of a fourth actuation mechanism of the bunion correction guide system of FIG. 4;

FIGS. 49A-49F depicts different plan views of one embodiment of a DMAA pin or screw of the fourth actuation mechanism of FIG. 45;

FIGS. 50A-50E depicts different plan views a knob of the fourth actuation mechanism of FIG. 45;

FIGS. 51A-51E depicts different plan views a knob of the fourth actuation mechanism of FIGS. 48A-48D;

FIG. 52 depicts an isometric view of a fixation guide arm assembly of the bunion correction guide system of FIGS. 1A-1B;

FIG. 53 depicts an isometric view of a fixation guide assembly of the bunion correction guide system of FIGS. 2A-2B;

FIGS. 54A-54B depicts isometric views of a fixation guide assembly and alignment column assembly of the bunion guide correction system of FIGS. 3A-3E;

FIGS. 55A-55E depicts various plan views of a guide arm of the fixation guide arm assembly of FIG. 52;

FIGS. 56A-56H depicts various plan views of a guide arm of the fixation guide arm assembly of FIGS. 54A-54B;

FIGS. 57A-57F depicts different plan views of one embodiment of a sleeve jig of the fixation guide arm assembly of FIG. 52;

FIGS. 58A-58E depicts different plan views of a sleeve jig of the fixation guide arm assembly of FIGS. 54A-54B;

FIGS. 59A-59C depicts different plan views of one embodiment of a drill sleeve of the fixation guide arm assembly of FIGS. 52 and 53;

FIGS. 60A-60B depict isometric views of an alignment column assembly of the fixation guide assembly of the bunion correction guide system of FIGS. 3A-3B;

FIGS. 61A-61H depict various views of a first alignment column of the alignment column assembly of FIGS. 60A-60B;

FIGS. 62A-62H depict various views of one embodiment of a second alignment column of the alignment column assembly of FIGS. 54A-54B;

FIG. 63A illustrates a flowchart for one embodiment for a method of correcting a bunion deformity;

FIG. 63B illustrates a flowchart for an alternative embodiment for a method of correcting a bunion deformity;

FIG. 64 illustrates a bunion deformity with various angular deformity measurements;

FIGS. 65A-65E illustrates the step of aligning the bunion correction guide system;

FIGS. 66A-66B illustrates the step of moving the capital fragment in a first direction and motion;

FIGS. 67A-67B illustrates the step of moving the capital fragment in a second direction and motion;

FIGS. 68A-68C illustrates the step of moving the capital fragment in a third direction and motion;

FIGS. 69A-69B illustrates the step of moving the capital fragment in a fourth direction and motion;

FIGS. 70A-70C illustrates the step of stabilizing the capital fragment in its final position;

FIG. 71A-71B depicts flowcharts with different embodiments of a method of fixation using the Fixation Guide Assembly of the bunion correction guide system of FIGS. 3A-3E;

FIGS. 72A-72J depicts the method of fixation using the Alignment Column Assembly and the Fixation Guide Assembly of the bunion correction guide system of FIGS. 3A-3E;

FIGS. 73A-73C depicts bunion guide sterile packaging kit; and

FIGS. 74A-74C depicts a method of loading and storing a bunion guide into a packaging kit;

### BRIEF SUMMARY OF INVENTION

In one embodiment, a method of correcting a hallux valgus using the bunion guide correction system comprises the steps of: aligning a bunion guide system to a first axis and a second axis on a portion of the outer surface of the foot, the foot includes a metatarsal having a capital fragment and a metatarsal body, the metatarsal body including a metatarsal body longitudinal axis; resecting the capital fragment from the metatarsal body; distracting the metatarsal body from the capital fragment in a first direction using a first actuation mechanism of the bunion guide system; rotating the capital fragment in a second direction using a second actuation mechanism of the bunion guide system; displacing the capital fragment in a third direction using a third actuation mechanism of the bunion guide system; rotating the capital fragment in a fourth direction using a fourth actuation mechanism of the bunion guide assembly to obtain a final corrected position; and stabilizing the capital fragment in the final corrected position.

In another embodiment, the bunion guide correction system comprises: an angled base plate; a first actuator mechanism, the first actuator mechanism comprises a tarsal base and a distraction screw, at least a portion of the distraction screw being fixed to the angled base plate, at least a portion of the distraction screw extending through a portion of the base, the base being movable relative to the distraction screw; a second actuator mechanism, the second actuator mechanism comprises an arc rack, a gear knob and a rotational block, at least a portion of the arc rack extends through the rotational block, the gear knob engages with the arc rack to allow the arc rack to be movable relative to the rotation knob; a third actuator mechanism, the third actuator mechanism comprises a housing and a translation screw, at least a portion of the translation screw being fixed to the angled base plate, at least a portion of the translation screw extending through the housing, the housing being movable relative to the translation screw; and a fourth actuator mechanism, the fourth actuator mechanism comprises a pin and knob, the pin is inserted through at least one opening on the housing of the third actuator mechanism, the pin engages with a portion of the arc rack of the second actuator mechanism to allow the arc rack to be movable relative to the angled base plate.

### DETAILED DESCRIPTION OF THE INVENTION

Therefore, a need remains for a bunion correction guide system and a method of use that is more effective in correcting bunion conditions because it can be performed as a minimally invasive procedure, thus reducing discomfort, scarring and recovery time in comparison to other bunion correction treatments. Furthermore, the bunion correction guide system also should provide repeatability from patient-to-patient and substantially proper anatomical alignment of the afflicted portion of bone or joint.

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without these specific details. In other instances, well-known structures are not shown in detail in order to avoid unnecessary obscuring of the present invention.

Thus, all the implementations described below are exemplary implementations provided to enable persons skilled in the art to make or use the embodiments of the disclosure and are not intended to limit the scope of the disclosure, which is defined by the claims. As used herein, the word “exemplary” or “illustrative” means “serving as an example, instance, or illustration.” Any implementation described herein as “exemplary” or “illustrative” is not necessarily to be construed as preferred or advantageous over other implementations.

The following description references systems, methods, and apparatuses for orthopedic surgery involving a foot or lower extremities. However, those possessing an ordinary level of skill in the relevant art will appreciate that other extremities, joints, other metatarsals (second metatarsal through the fifth metatarsal) and parts of the musculoskeletal system are suitable for use with the foregoing systems, methods and apparatuses. Likewise, the various figures, steps, procedures and work-flows are presented only as an example and in no way limit the systems, methods or apparatuses described to performing their respective tasks or outcomes in different time-frames or orders. The teachings of the present invention may be applied to any orthopedic surgery, such as on the hand as well as other upper and lower extremities and may be implemented in other treatments sites that have similar anatomical considerations.

The invention disclosed herein describes a bunion correction guide system or bunion guide system that facilitates a percutaneous, minimally invasive surgical technique. The bunion guide system) is designed to multi-directionally control or align the first metatarsal capital fragment (distal articular portion of the first metatarsal or metatarsal head) to its desired or proper anatomical position using different mechanical actuation mechanisms. Accordingly, the invention disclosed herein further discloses stabilizing the first metatarsal capital fragment in its desired or proper anatomical position via one or more fixation screws once the bunion correction guide system has facilitated the desired or final anatomical positioning or alignment. With the bunion correction guide system multi-directional or multi-planar capabilities, it can improve hallux valgus corrections into a more consistent, repeatable, and efficient procedure with reduced recovery time.

The bunion correction guide system or the bunion guide system is desirably disposed over a patient's foot or is positioned on a portion of the superior surface of a patient's foot 55. The bunion guide system's positioning on the foot surface aids in stability during the use or control of the various actuation mechanisms; reduction of forces during use or control of the actuation mechanisms; facilitates more accurate alignment of head of the first metatarsal; and reduces the surgical working coverage area. Accordingly, the bunion correction guide comprises a plurality of fixed or modular actuation mechanisms that may be controlled, adapted or directed at treating mild to moderate hallux valgus angle deformities by allowing successful correction of the deformity by: normalizing the tension of the tendons and muscles; establishing a congruous MTP joint, maintaining or increasing the first MTP joint range of motion; reducing the intermetatarsal angle (IMA); realigning the sesamoids underneath the metatarsal head to restore the ability to bear weight and distribute load properly; and/or realigning the hallux to a rectus position. Accordingly, the ultimate goal is the elimination of pain, restoration of a congruous metatarsophalangeal joint, realignment of the hallux into its proper anatomical position (including the rotation of sesamoids), and preservation of joint motion.

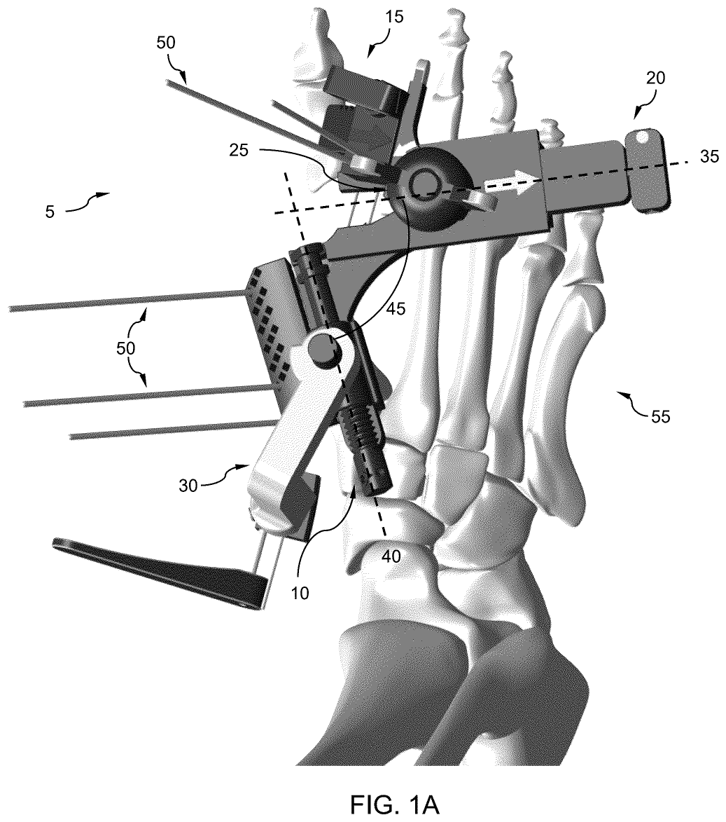

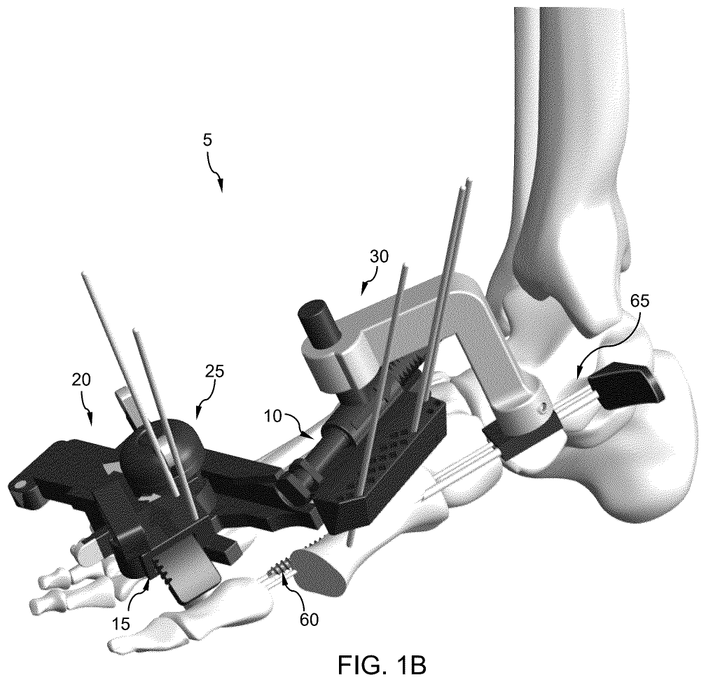

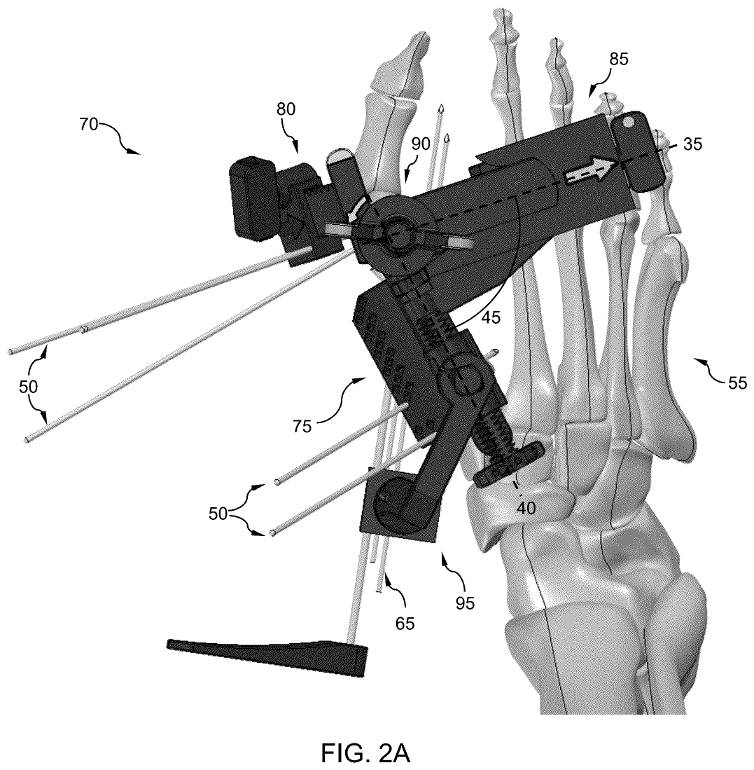

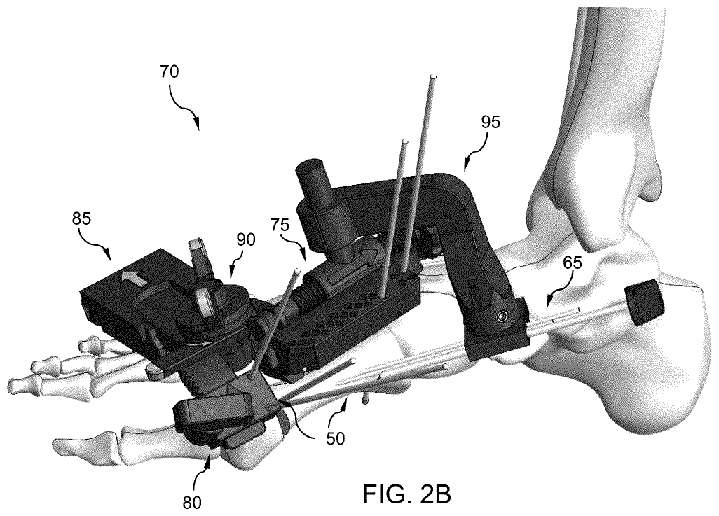

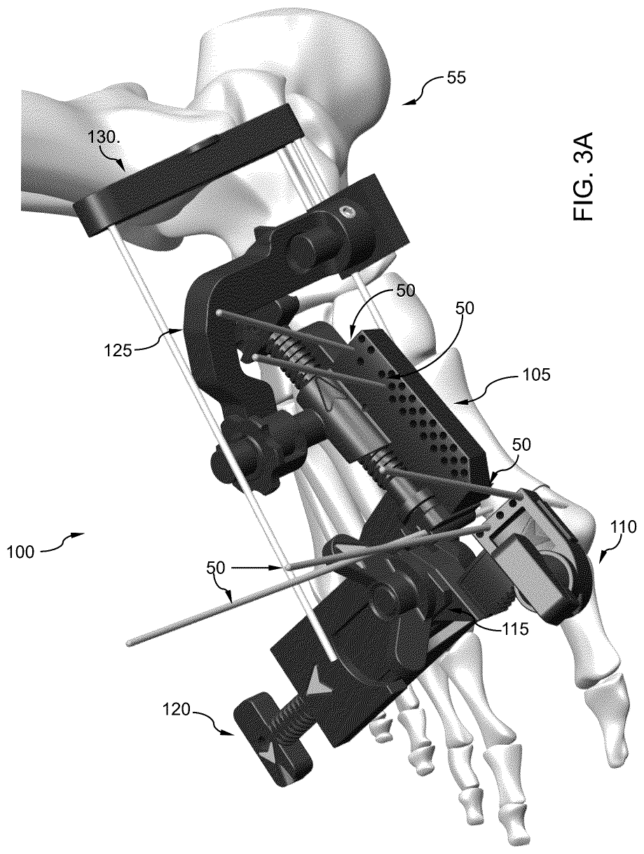

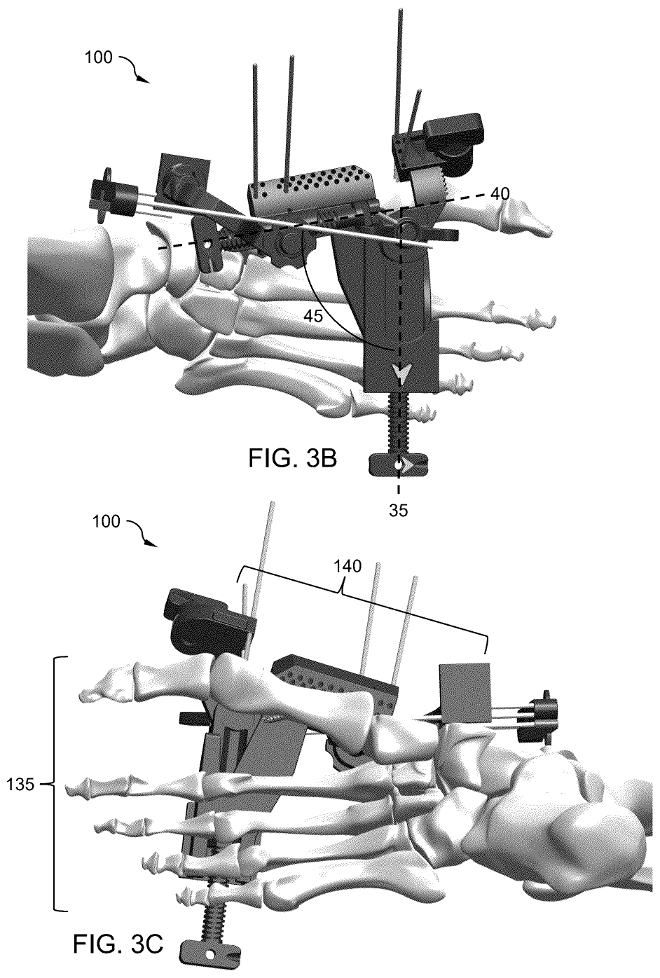

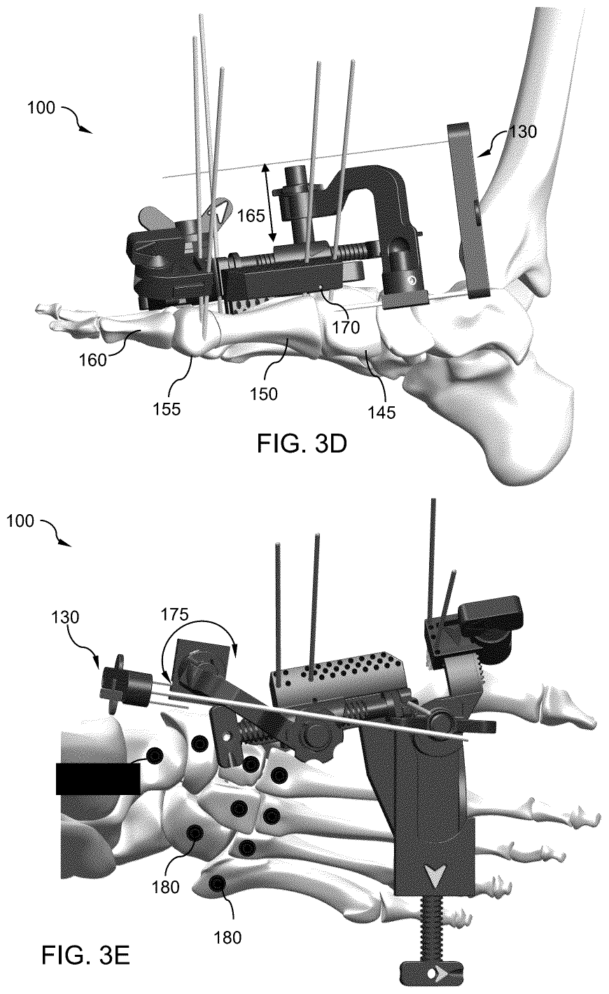

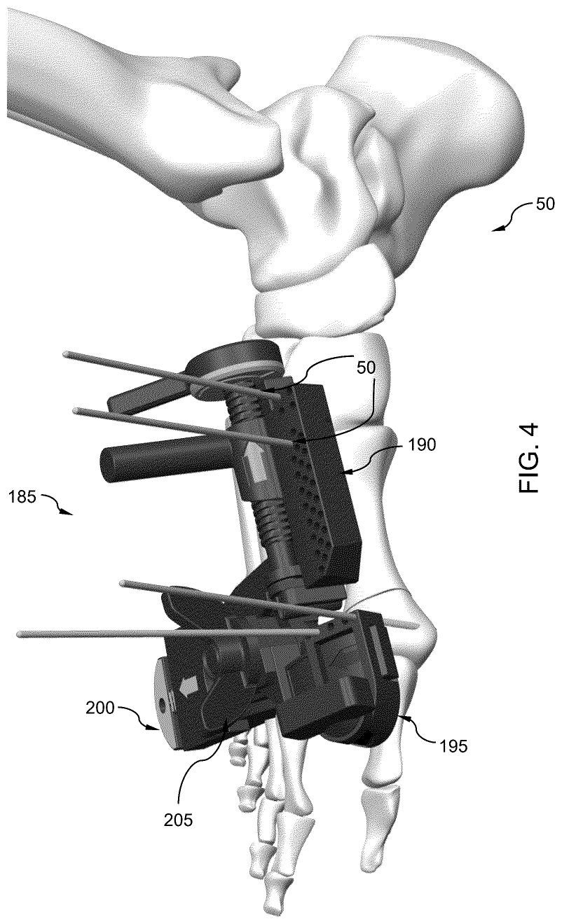

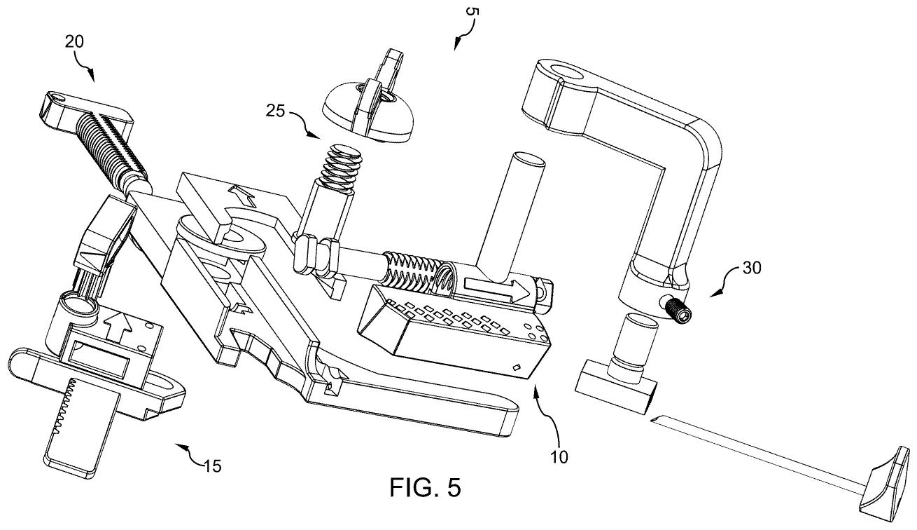

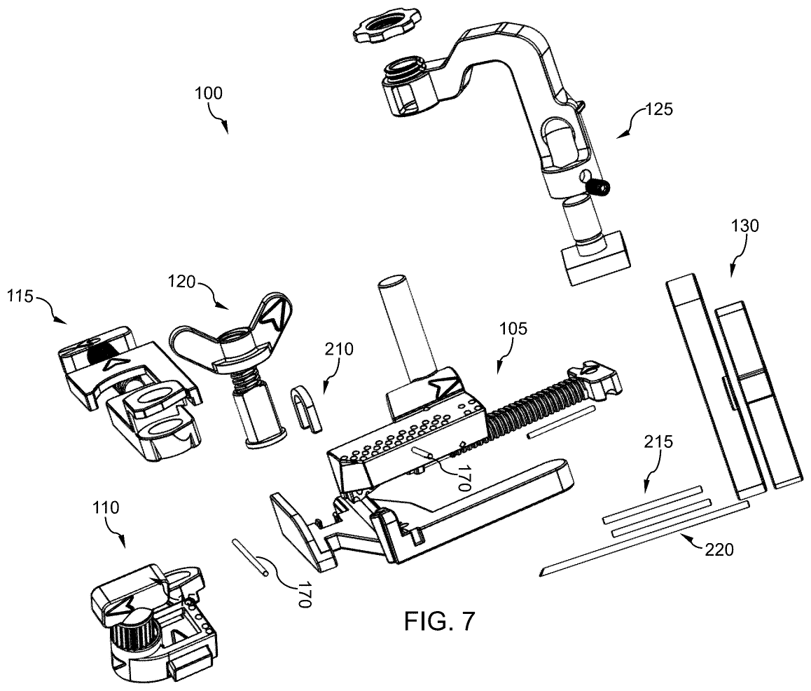

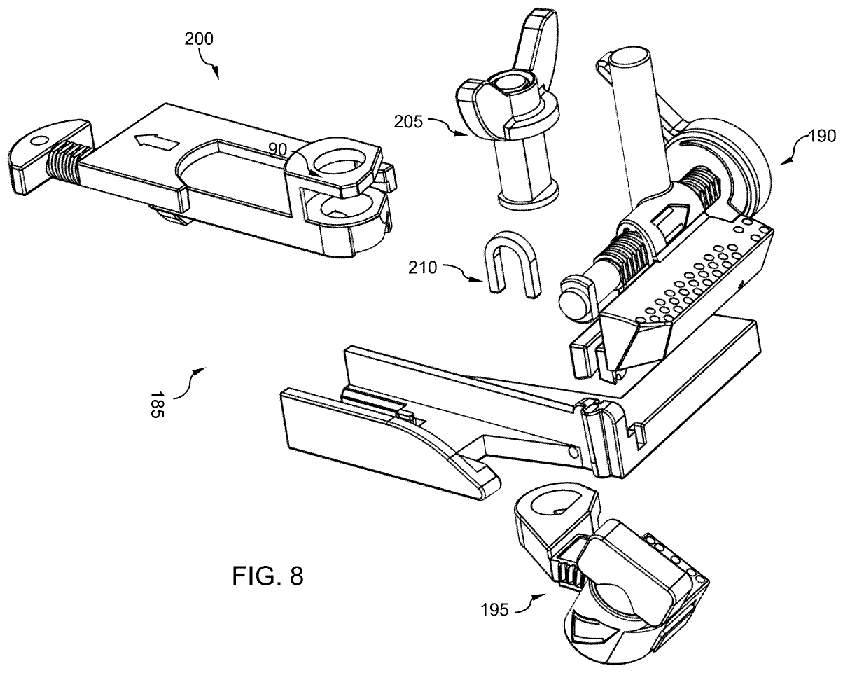

FIGS. 1A-1B, 2A-2B, 3A-3E, 4 and 5-8 depict isometric views and exploded views of a first embodiment 5, a second embodiment 70, a third embodiment 100, and a fourth embodiment 185 of a bunion guide system, respectively. The bunion guide system 5, 70, 100, 185 may comprise at least four different actuator mechanisms having at least four different motions and a fixation guide arm assembly 30, 95, 125. The bunion guide system 5, 70, 100, 185 may further comprise an alignment column assembly 130. The bunion guide system 5, 70, 100, 185 further comprises one or more anchoring pins 50, one or more radiopaque markers or pins 170, one or more guide wires or pins 65, and/or any combination thereof. The bunion guide system 5, 70, 100, 185 further comprises one or more fixation screws 60.

The at least four actuator mechanisms comprise a first actuator mechanism 10, 75, 105, 190, a second actuator mechanism 15, 80, 110, 195, a third actuator mechanism 20, 85, 115, 200, and a fourth actuator mechanism 25, 90, 120, 205. Each of the first actuator mechanisms 10, 75, 105, 190, a second actuator mechanism 15, 80, 110, 195, a third actuator mechanism 20, 85, 115, 200, and a fourth actuator mechanism 25, 90, 120, 205 are engaged to the angled base plate 235, 250, 265, 280 to allow specific uniaxial or uniplanar movement or motion, thus allowing movement in one axis or plane, respectively. Each of the actuator's mechanisms may be modular and removably connected or fixed into a single system. Each of the different embodiments of the actuator mechanisms presented with the different bunion guide system embodiments 5, 70, 100, 185 can easily be adapted to fit, engage or be combined with any of the bunion guide system embodiments 5, 70, 100, 185.

The first actuator mechanism 10, 75, 105, 190, a second actuator mechanism 15, 80, 110, 195, a third actuator mechanism 20, 85, 115, 200, and a fourth actuator mechanism 25, 90, 120, 205 enable the bunion guide system to control the motion of the capital fragment 155 and/or great toe in 4 dimensions (4D) to achieve the final positional and cosmetic alignment of the great toe. Each of the first actuator mechanism 10, 75, 105, 190, a second actuator mechanism 15, 80, 110, 195, a third actuator mechanism 20, 85, 115, 200, and a fourth actuator mechanism 25, 90, 120, 205 are modular and/or fixed, and when assembled together onto the angled base plate 235, 250, 265, 280 in a first longitudinal axis 40 and a second transverse axis 35 to form an inherent angle 45, D, to ensure that the movement or mobilization of the capital fragment 155 (e.g., the head of the first metatarsal) is coaxially aligned and/or substantially coaxially aligned with the toe joints (proximal phalange 160 and the body of the first metatarsal 150). Otherwise, without the inherent angle, lengthening or shortening of the attached muscles and/or tendons to the first metatarsal may occur, thus reducing ability to produce or sustain tension forces.

The motions of the first actuator mechanism 10, 75, 105, 190, a second actuator mechanism 15, 80, 110, 195, a third actuator mechanism 20, 85, 115, 200, and a fourth actuator mechanism 25, 90, 120, 205, respectively, include distraction of the capital fragment 155 (distraction-compression of the capital fragment) from the body of the first metatarsal 150; rotation of the capital fragment 155; displacement or translation of the capital fragment 155; and the rotation or de-rotation of proximal articular set angle (PASA) or the distal metatarsal articular angle (DMMA) of the capital fragment 155 for final cosmetic alignment of the Great Toe to its desired anatomical position.

Furthermore, the bunion guide systems 5, 70, 100, 185, have a desired low profile to be able to be positioned over the foot 55. The bunion guide system 5, 70, 100, 185, comprises a guide system width 135 that may be equal to and/or substantially equal to a width of a patient's foot 55. Accordingly, the bunion guide systems 5, 70, 100, 185 comprises a guide system length 140 that may be equal to and/or substantially equal to the length of a patient's foot 55. At least a portion of the guide system length 140 may extend onto the cuneiform bones 145. Also, at least a portion of the bunion guide systems 5, 70, 100, 185 is intended to anchored to at least one of the cuneiform bones 145, it may be anchored to other cuneiform positions 180 of the cuneiform bones 145 to correct other metatarsals, the 2^{nd} metatarsal to the 5^{th} metatarsal.

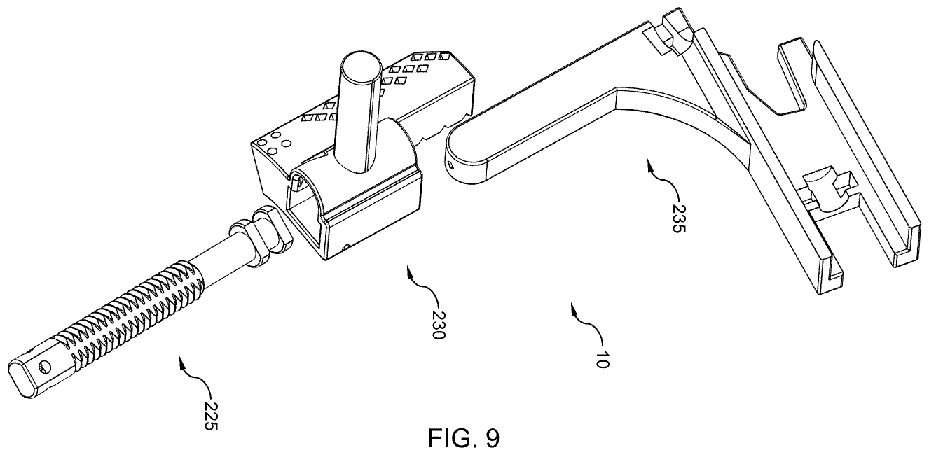

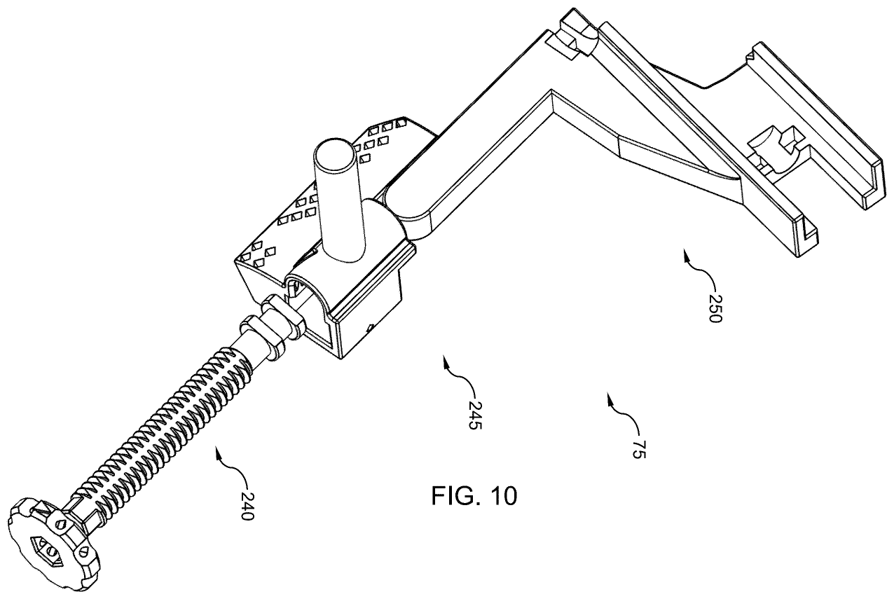

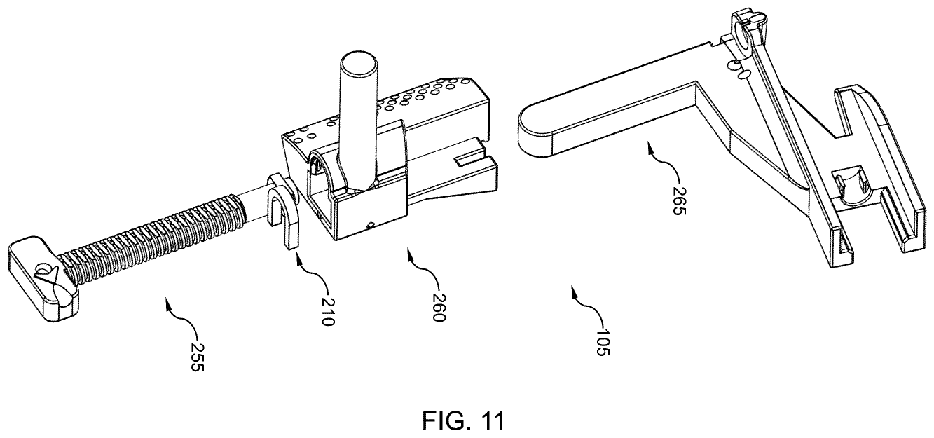

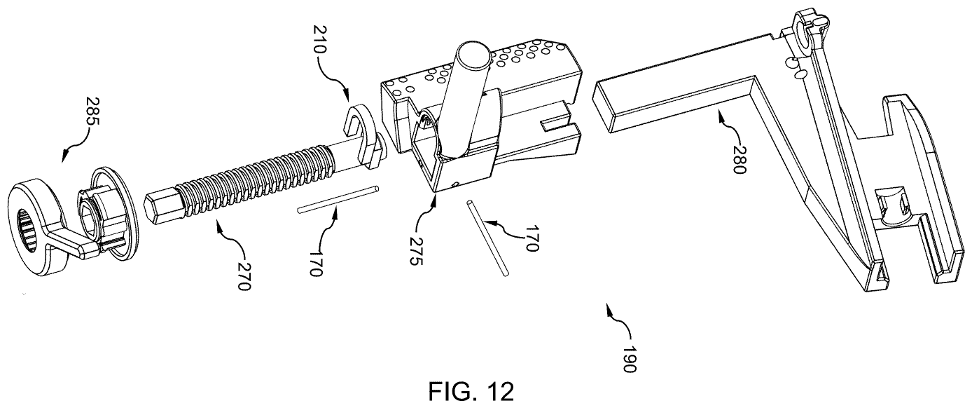

FIGS. 9-12 illustrates exploded isometric views of a first actuator mechanism 10, 75, 105, 190 of the bunion guide system 5, 70, 100, 185. The first actuator mechanism 10, 75, 105, 190 of the bunion guide system 5, 70, 100, 185 comprises an angled base plate 235, 250, 265, 280, a tarsal base 230, 245, 260, 275, and a distraction screw 225, 240, 255, 270. The first actuator mechanism 10, 75, 105, 190 may further comprise radiopaque pins or markers 170.

The first actuator mechanism 10, 75, 105, 190 is a mechanical linear actuator that converts rotational motion into linear motion. Its operation relies on the movement or rotation of distraction screw relative to the tarsal base along the first metatarsal body longitudinal axis. Alternatively, by rotating the distraction screw, the tarsal base moves relative to the distraction screw and angled base plate along the first metatarsal body longitudinal axis. The movement allows the distraction of the first metatarsal head from the body of the first metatarsal to a desired distance. The desired distance may comprise 0.25 mm to 5 mm; distance may include 0.5 mm to 3 mm; distance may include 1 mm to 3 mm; and/or the distance may be at least 1 mm or greater. The rotation may comprise clockwise or counterclockwise rotation. In one embodiment of the first actuator mechanism, the actuator mechanism facilitates distraction-compression of the capital fragment. The direction comprises movement substantially along the longitudinal axis of the first metatarsal towards the posterior direction and/or anterior towards the posterior direction. The first actuator mechanism comprises the angled base plate, the tarsal base and the distraction screw. The first actuator mechanism further comprises a securing element.

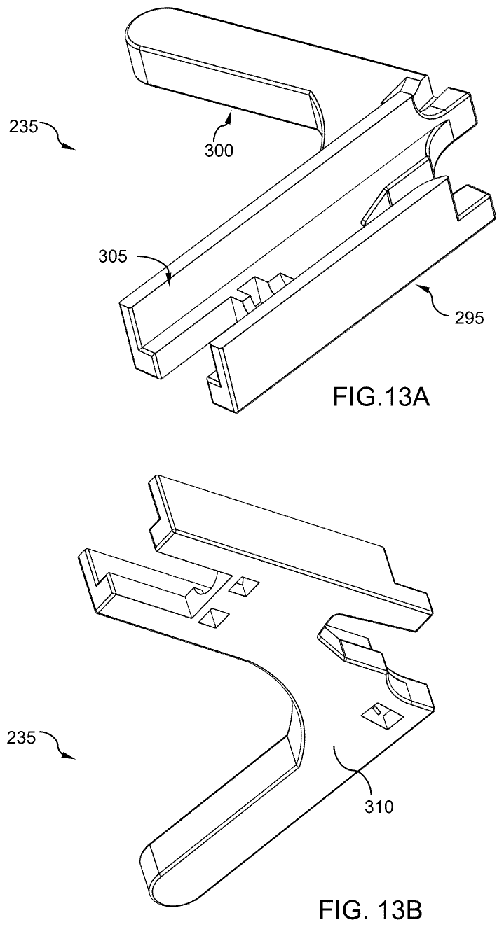

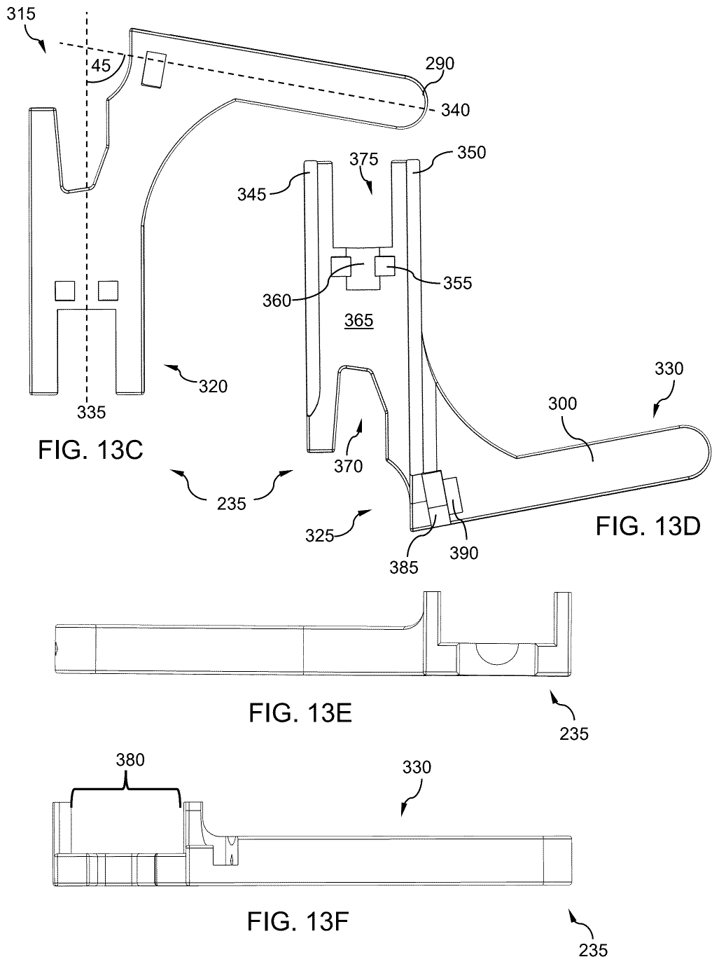

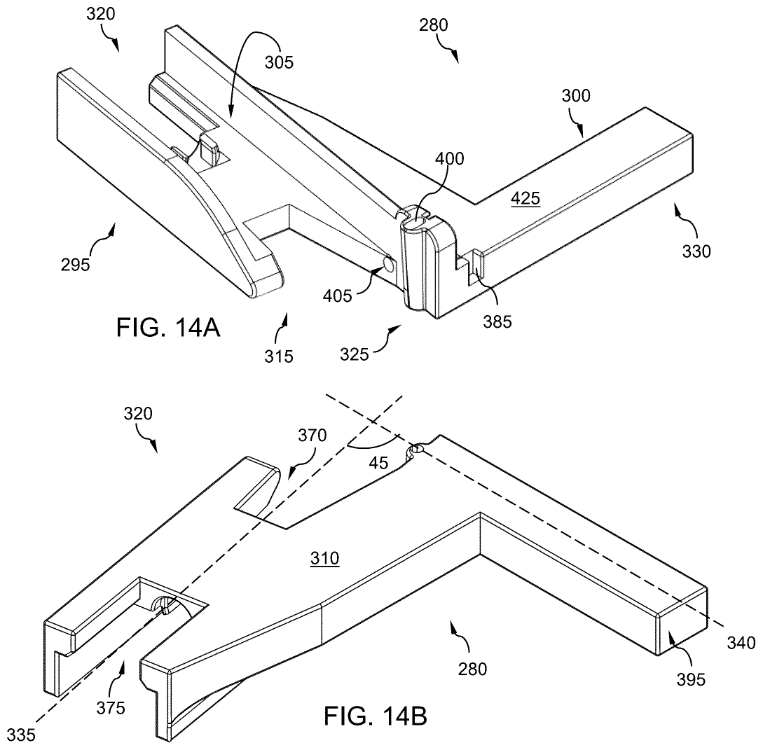

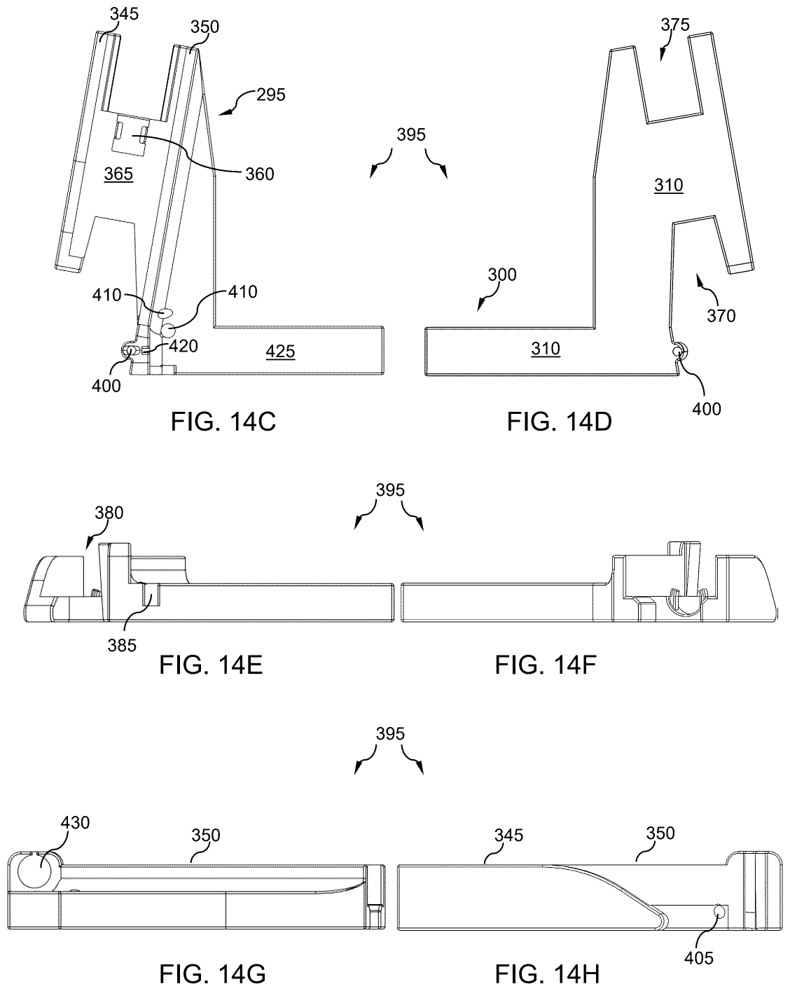

FIGS. 13A-13F and 14A-14H illustrate multiple plan views of different embodiments of an angled base plate 235, 280 of the first actuation or actuator mechanism 10, 75, 105, 190. The angled base plate 235, 250, 265, 280 comprises a first portion 290 and a second portion 295. The first portion 290 is coupled or affixed to the second portion 295. The first portion 290 is coupled to the second portion 295 at an angle 45, D. Angle 45, D, may comprise at least 75 degrees or greater and/or 80 degrees or greater.

The first portion 290 comprises a first portion longitudinal axis 335, a first or guide slot 370, a second slot 375 and a track, channel or rail 305. The first portion 290 further comprises a first end 315 and a second end 320. The guide slot or first slot is positioned adjacent to the first end 315. The guide slot 370 comprises a slot width and a slot length that creates a “viewing window.” The slot width and slot length may be sized and configured to accommodate the viewing of the first end or distal end of the metatarsal that includes at least a portion of the metatarsal head (e.g., capital fragment) and the metaphalangeal joint and/or any surgical instrumentation. This viewing window comprising the slot width and slot length of the first slot may guide the physician to perform the necessary osteotomy to distract the capital fragment from the body of the first metatarsal. The first slot is positioned at, near or adjacent to the first end 315 of the first portion 290 and the second slot is positioned at, near or adjacent to the second end 320 of the first portion 290.

The first portion 290 of the angled base plate 235, 250, 265, 280 may further comprise a track 305. The track 305 comprises a first track wall 345, a second track wall 350, a track surface 365, a track channel 380 and a track length. The track channel and/or the track width 380 is sized and configured to receive a portion of a displacement or translational screw housing 1015 of the third actuation mechanism 20, 85, 115, 200. Alternatively, the track and/or the track width 380 is sized and configured to receive a portion of the width of the displacement screw housing 1015. The displacement screw housing 1015 can move or slide relative to the angled base plate 235, 250, 265, 280. The track length extends between the first end 315 to the second end 320 of the first portion 290. The track surface 365 is disposed between the first track wall 345 and second track wall 350. The first 345 and second track wall 350 extend upwardly from the track surface 365. Alternatively, the first track wall 345 and second track wall 350 extend perpendicular from the track surface 365.

The track surface 365 may further comprise a recess 360. The recess 360 is sized and configured to receive a portion of the translation screw 1020 of the third actuator mechanism 20, 85, 115, 200. The recess 360 is sized and configured to receive a portion a second end or tail end 1140 of the translation screw 1020. The recess 360 may be positioned or disposed near or adjacent to the first end 315 or the second end 320 of the first portion 295. Once the second end or tail end 1140 of the translation screw 1020 is disposed within the recess 360, this allows the translational screw 1020 to be fixed relative to the angled base plate 235, 250, 265, 280. Rotation of the translational screw 1020 forces the translational housing 1015 to move relative to the translation screw 1020 and the angled base plate 235, 250, 265, 280. The direction of movement is from medial to lateral direction or along the first portion longitudinal axis, the first portion longitudinal axis may be substantially transverse to the second portion longitudinal axis. The track surface 365 may further comprise one or more openings 355. Each of the one or more openings 355 are positioned adjacent to the recess 360 of the track surface 365. In one embodiment, the track surface 365 may comprise a recess 360, a first opening and a second opening 355. The recess 360 is positioned between the first opening and second opening.

The second portion 300 comprises a first end 325 and a second end 330. The first end 325 of the second portion 300 is coupled to the first end 315 of the first portion 295 at an orientation or angle, D. The first end of the second portion comprises a recess. The recess is sized and configured to receive a portion of a distraction screw second end and/or tail end. At least a portion of the longitudinal member of the second portion is disposed within or inserted into a tunnel of the tarsal base. Alternatively, at least a portion of the second portion is inserted into the tunnel of the tarsal base. At least a portion of the second portion is inserted into the bottom portion of the tunnel of the tarsal base. The tarsal base may move or slide relative to the angled base plate.

The second portion may further comprise a first recess. The first recess is sized and configured to receive a portion of the distraction screw of the first actuator mechanism. The recess is sized and configured to receive a portion a second end or tail end of the distraction screw. The recess may be positioned or disposed near or adjacent to the first end of the second portion. Once the second end or tail end of the distraction screw is disposed within the recess, this allows the distraction screw to be fixed relative to the angled base plate. The first recess may extend from a top surface of the angled base plate towards the bottom surface of the angled base plate. Alternatively, the first recess may extend upwardly from the top surface of the angled base plate. The first recess may extend perpendicular to the top surface of the angled base plate.

The second portion may further comprise a second recess or a retention element opening. The second recess or retention opening is sized and configured to receive a portion of a retention element. The retention element will be disposed over a portion of the second end or tail end of the distraction screw and inserted into the second recess or the retention element opening to secure the distraction screw to the angled base plate, as well as reduce movement or migration during activation of the first actuator mechanism.

The second portion may further comprise one or more openings. The one or more openings may be positioned near or adjacent to the first end of the first portion of the angled base plate. The one or more openings are sized and configured to receive a portion of one or more anchoring pins. The one or more openings comprise a longitudinal opening axis. Each of the longitudinal opening axis may be different or the same. Each of the one or more openings are spaced apart. In another embodiment, the second portion comprises a first opening having a first opening longitudinal axis and a second opening with a second longitudinal axis. The first opening is spaced apart from the second opening. The first longitudinal axis of the first opening is orthogonal to the longitudinal axis of the first portion. The second longitudinal axis of the second opening is oblique to the longitudinal axis of the first portion. The second longitudinal axis of the second opening is oriented at an angle.

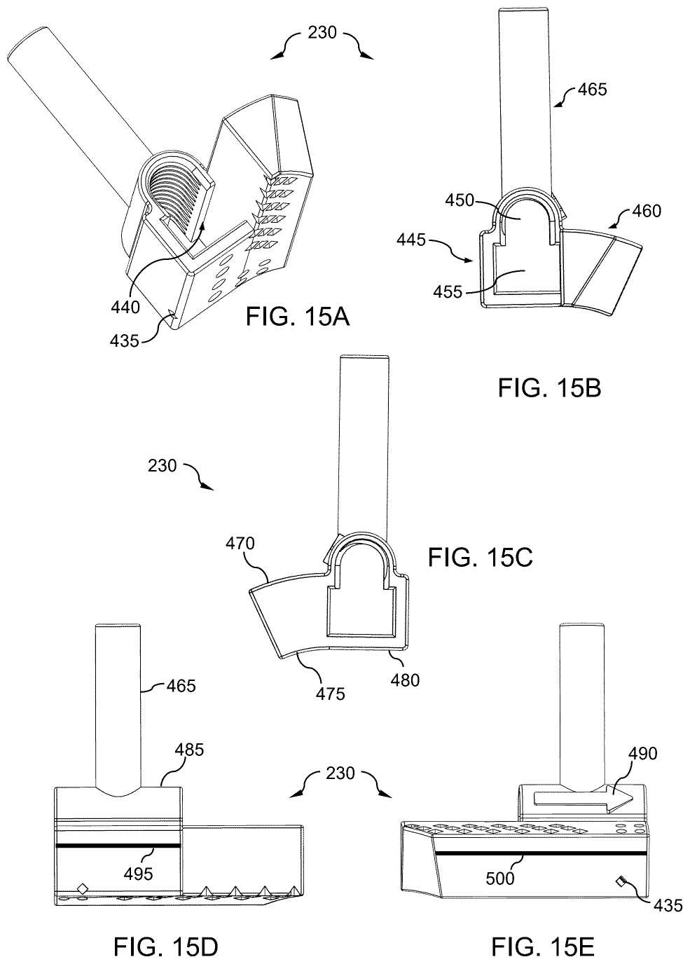

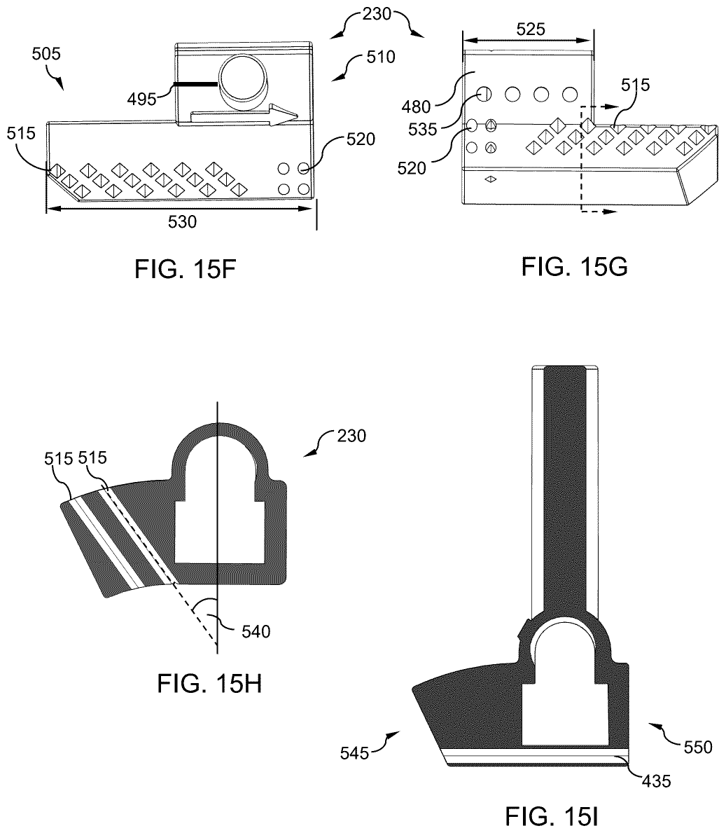

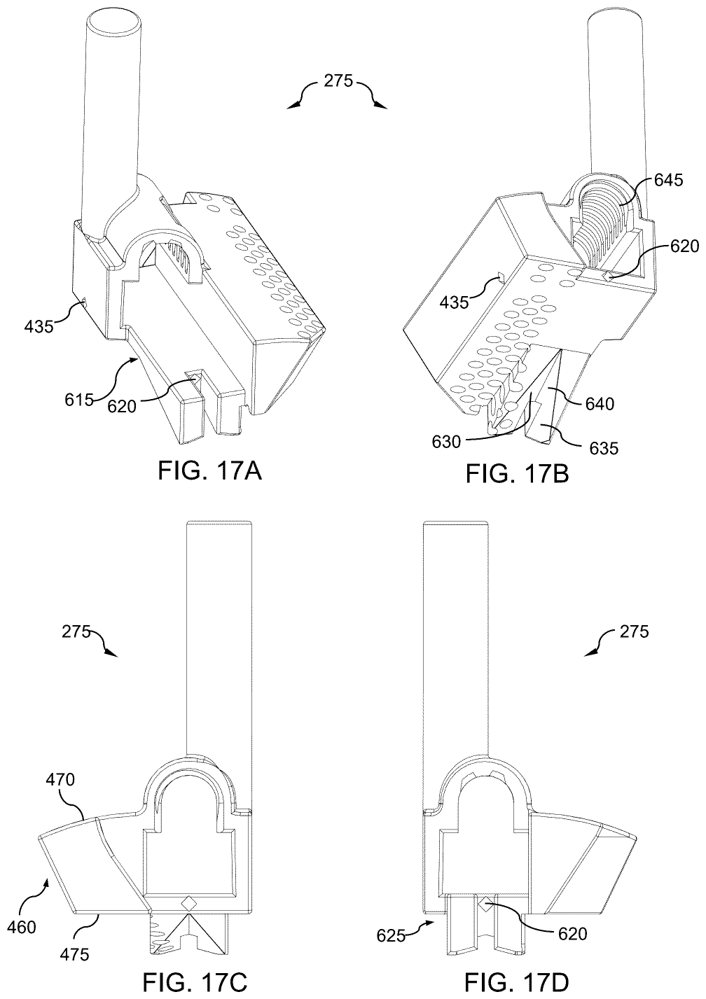

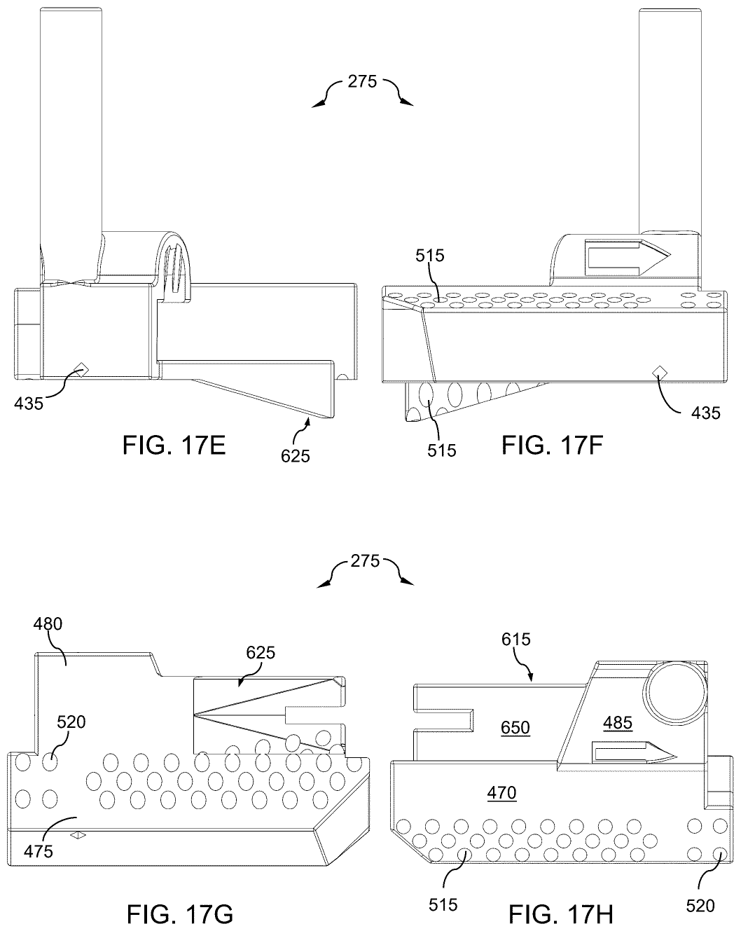

FIGS. 15A-15I, 16A-16G and 17A-17H illustrate different plan views of different embodiments of a tarsal base 230, 275 of the first actuation mechanism 10, 75, 105, 190. The tarsal base 230, 245, 260, 275 comprises a stem or post, a tunnel housing, and an anchor pin body or an anchor pin body extension. The tunnel housing is coupled to the anchor pin body. The tunnel housing is fixed to the anchor pin body.

The tunnel housing of the tarsal base comprises a tunnel or passageway, a top surface, a bottom surface, a first end and a second end. The tunnel or passageway further comprises a top or first portion and a bottom portion or a second portion and a tunnel or passageway length. The top portion of the tunnel or passageway is sized and configured to receive a portion of the distraction screw and/or the body of the distraction screw. A portion of the tunnel or passageway and/or a top portion of the tunnel or passageway may further comprise threads. The first portion or the top portion of the tunnel or passageway comprises a shape, the shape is a semi-circle, convex, arch or arcuate shape. The tunnel or passageway may extend from a first end to a second end of the tarsal base or the tunnel housing.

The bottom portion of the tunnel is sized and configured to receive a portion of the 2nd portion of the angled base plate and/or the longitudinal member of the 2nd portion of the angled base plate. The bottom portion of the tunnel allows the tarsal base to move relative to the tarsal base plate while the physician rotates the distraction screw. The movement of the tarsal base is parallel or substantially parallel to the longitudinal axis of the first metatarsal. More specifically, the angled based plate is fixed to the bone to a patient and the distraction screw is fixed to the angled base plate to allow the tarsal base to move relative to the angled base plate to a desired distraction distance while the physician rotates the distraction screw. The rotation may occur clockwise or counterclockwise. The distraction distance may comprise at least 0.25 mm; the distraction distance may comprise at least 0.5 mm; the distraction distance may comprise at least 1 mm; the distraction distance may comprise 1.5 mm; the distraction distance may comprise at least 2 mm; and/or the distraction distance may comprise a range of 1 mm to 5 mm. A width of the first portion of the tunnel housing may be smaller than the width of the second portion of the tunnel housing.

The tarsal base and/or the tunnel housing comprises a radiopaque marker opening. The radiopaque marker opening is sized and configured to receive a radiopaque pin, wire or marker. The radiopaque marker opening extends from a side of the tarsal base and/or the tunnel housing towards the center of the tarsal base. The radiopaque marker opening extends from a side of the tarsal base and/or the tunnel housing towards the anchor pin body extension. The radiopaque marker opening of the tunnel housing is transverse to the first radiopaque marker opening of the anchor pin body extension. The radiopaque marker opening of the

The anchor pin body extension comprises an extension length, and extension width and/or an extension shape, a top surface and a bottom surface. The top surface of the anchor body extension comprises a curved surface, an arch or arcuate surface. The bottom surface may comprise a flat, planar surface, an arch or arcuate, or concave surface. The anchor body extension length is longer than the tunnel or passageway length and/or length of the tunnel housing. The top surface shape of the anchor pin body extension may have the same shape as the bottom surface shape of the anchor pin body extension. The top surface of the anchor pin body extension may have a different shape than the bottom surface shape of the anchor pin body extension.

The anchor pin body extension further comprises a plurality of holes or anchor openings. The plurality of holes may extend from a top surface of the body extension through the bottom surface of the body extension. The plurality of holes or anchor openings comprise an inner diameter or inner width. The inner diameter or width of the plurality of holes or anchor openings are sized and configured to receive a portion of one or more drill wires and/or anchor pins. The plurality of holes or anchor openings comprise an angled trajectory or orientation, a, due to the arch or arc shape of the body extension. The angle of the anchor openings may comprise 0 degrees to 60 degrees; the angle may comprise 15 degrees to 60 degrees; the angle may comprise 15 degrees to 45 degrees. The angle may comprise less than or equal to 60 degrees or less than or equal to 45 degrees. The angle may comprise greater than or equal to 15 degrees or 30 degrees. The drill wires may comprise K-wires. The K-wires may comprise threaded K-wires.

In another embodiment, the anchor pin body extension comprises a first plurality of openings with a first plurality of openings longitudinal axis and a second plurality of openings with a second plurality of openings longitudinal axis. The first plurality openings are spaced apart from the second plurality of openings. Each of the first plurality of openings extends from a top surface through the bottom surface of the anchor pin body extension. Each of the first plurality of openings longitudinal axis comprise a first plurality of orientations. Each of the first plurality of orientations of the first plurality of longitudinal axis may be the same or different. The first plurality orientations and/or the second plurality of orientations may comprise 0 degrees to 60 degrees; the orientation angle may comprise 15 degrees to 60 degrees; the orientation angle may comprise 15 degrees to 45 degrees. The orientation angle may comprise less than or equal to 60 degrees or less than or equal to 45 degrees. The first plurality of openings is disposed near the first end of the tarsal base or the first end of the anchor pin body extension and extend towards the second end of the tarsal base or the second end of the anchor pin body extension. The first plurality of openings is positioned over the body of the first metatarsal. The first plurality of openings is positioned to align along, follow or be parallel the longitudinal axis of the body of the first metatarsal. Each of the first plurality of openings are positioned into a plurality of rows, each of the plurality of rows of the plurality of openings are parallel to each other.

Accordingly, the first plurality of openings may be positioned to align along, follow and/or be parallel to a longitudinal axis of any of the bodies of the second metatarsal, third metatarsal, fourth metatarsal, and/or fifth metatarsal. The first plurality of openings may be positioned over the bodies of the second metatarsal, third metatarsal, fourth metatarsal, and/or fifth metatarsal. The first plurality of openings may be positioned over the bodies of the metatarsal bones, the metatarsal bones may include a first, a second, a third, a fourth, a fifth metatarsal bones and/or any combination thereof.

Each of the second plurality of openings extends from a top surface through the bottom surface of the anchor pin body extension. Each of the second plurality of openings longitudinal axis comprise a second plurality of orientations. Each of the second plurality of orientations of the second plurality of longitudinal axis may be the same or different. The first plurality orientations and/or the second plurality of orientations may comprise 0 degrees to 60 degrees; the orientation angle may comprise 15 degrees to 60 degrees; the orientation angle may comprise 15 degrees to 45 degrees. The orientation angle may comprise less than or equal to 60 degrees or less than or equal to 45 degrees. The second plurality of openings are disposed near or adjacent to the second end of the tarsal base and/or second end of the anchor pin body extension. The second plurality of openings are positioned to be distal to the body of the first metatarsal. The second plurality of openings are positioned over the cuneiform bones, the navicular bone and/or the cuboid bone. The cuneiform bones may comprise the medial cuneiform, the intermediate cuneiform and/or the lateral cuneiform.

The tarsal base and/or the anchor pin body extension may comprise a first opening or passageway. The first opening or passageway extends from a first end of the anchor pin body extension through the second end of the anchor pin body extension. The first opening is disposed near or adjacent to the bottom surface of the of the tarsal base and/or the bottom surface of the anchor pin body extension. The first opening or passageway follows, aligns and/or is parallel to the longitudinal axis of the body of the first metatarsal. The first opening or passageway extends the length of the anchor pin body extension. The first opening is sized and configured to receive a portion of a radiopaque marker, pin or wire. The radiopaque marker, pin or wire should comprise a material to allow radiographic visualization of the targeted areas for proper alignment over the foot during surgical procedures.

The tarsal base and/or the anchor pin body extension may comprise a second opening or passageway. The second opening or passageway may be sized and configured to receive a portion of a radiopaque marker, pin or wire. The second opening or passageway may extend from a first side of the tarsal base towards the second side of the tarsal base. The second opening or passageway may extend from a first side of the anchor pin body extension towards the second side or towards the tunnel housing. Alternatively, the second opening or passageway may extend from a first side of the anchor pin body extension towards the center and/or towards the tunnel housing of the tarsal base. The second marker opening may be disposed near the bottom surface of the tarsal base and/or the bottom surface of the anchor pin body extension. The second opening or passageway may be transverse to the first marker opening. The second opening or passageway may be parallel to the first marker opening. The second passageway may intersect with the first passageway. The second passageway may not intersect with the first passageway. The second passageway may be spaced apart from the first passageway.

The stem or post of the tarsal base comprises a vertical axis, a length and an outer diameter. The stem or post of the tarsal base engages with a portion of the fixation guide assembly. More specifically, the stem or post of the tarsal base engages with a guide arm of the fixation guide arm assembly. The engagement of the guide arm with the stem or post of the tarsal base allows movement relative to the tarsal base along the vertical axis or length and the rotational axis. The outer diameter of the stem or post is sized and configured to be disposed or inserted into an opening of the guide arm of the fixation guide assembly allowing or enabling the guide arm to be movable along the length of the post or stem and/or rotatable relative to the tarsal base.

The stem or post extends from a top surface of the tarsal base. The stem or post extends perpendicular to the top surface of the tarsal base. Alternatively, the stem or post extends at an angled orientation from the top surface of the tarsal base. The angled orientation may comprise 0.5 degrees to 20 degrees; the angled orientation may comprise 0.5 degrees to 15 degrees; the angled orientation may comprise 0.5 degrees to 10 degrees; and/or the angled orientation may comprise 0.5 degrees to 5 degrees.

In another embodiment, the stem or post extends from a top surface of the tunnel housing. The stem or post extends perpendicular to the top surface of the tunnel housing. The stem or post extends at an angled orientation from the top surface of the tunnel housing. The length of the stem or post comprises a range of 25 mm to 45 mm; the length of the stem or post comprises a range of 30 mm to 40 mm; the length of the stem or post comprises at least 30 mm; and/or the length of the step or post comprises at least 35 mm.

The tarsal base may further comprise a platform. The platform extends from the tunnel housing towards the first end of the tarsal base. The platform comprises a platform top surface and a platform bottom surface. The platform top surface is flat, planar. At least a portion of the platform bottom surface extends at an angled orientation from the bottom surface of the tarsal base. At least a portion of the bottom surface extends downwardly or inferiorly and at an angled orientation from the bottom surface of the tarsal base.

At least a portion of the platform bottom surface comprises a recessed saddle. The recessed saddle is sized and configured to receive a portion of a metatarsal or a body of the metatarsal. The metatarsal may comprise a first, second, third, fourth, fifth metatarsal and/or any combination thereof. In one exemplary embodiment, the recessed saddle is sized and configured to receive a portion of a first metatarsal and/or a portion of the body of the first metatarsal. The recessed saddle comprises a first saddle wall and a second saddle wall. The first saddle wall is oriented at a first angle and the second saddle wall is oriented at a second angle. The first saddle wall and the second saddle wall intersect to form a substantially triangular shape. The first saddle wall is a mirror image of the second saddle wall to form a substantially triangular shape.

The tarsal base may comprise one or more indicators. In one exemplary embodiment, the tarsal base comprises a first indicator, a second indicator and a third indicator. The first indicator comprises a directionality indicator. The second indicator and third indicator comprises an imaging indicator (not shown). The imaging indicator may comprise a radiolucent or radiopaque material. The imaging indicator may comprise one or more drill wires or anchor pins.

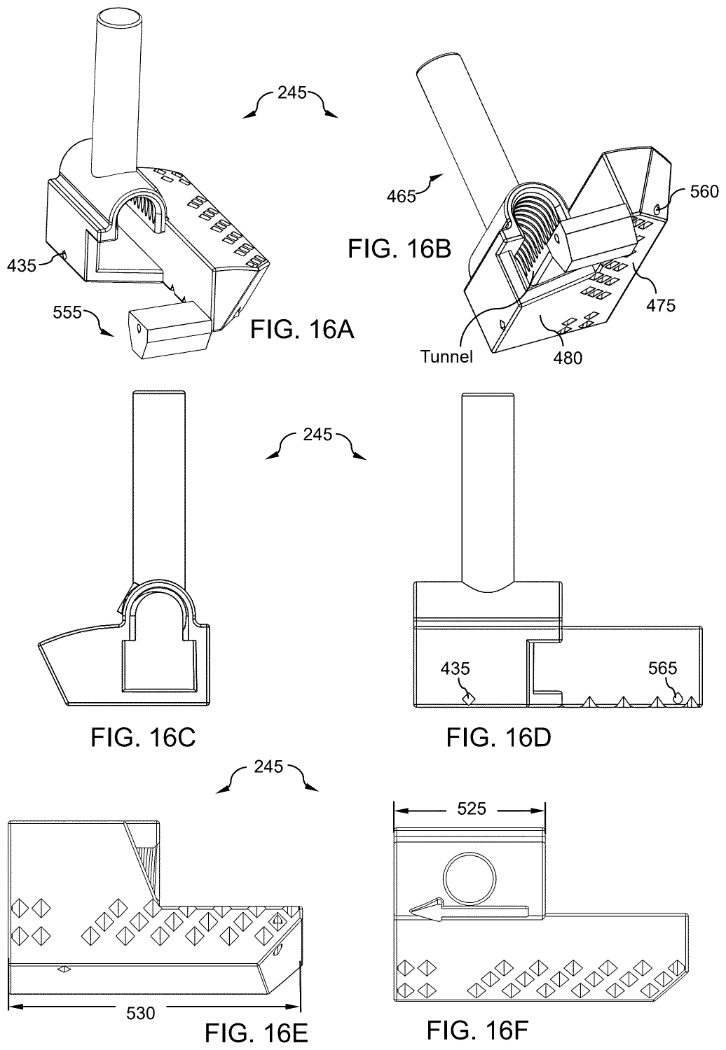

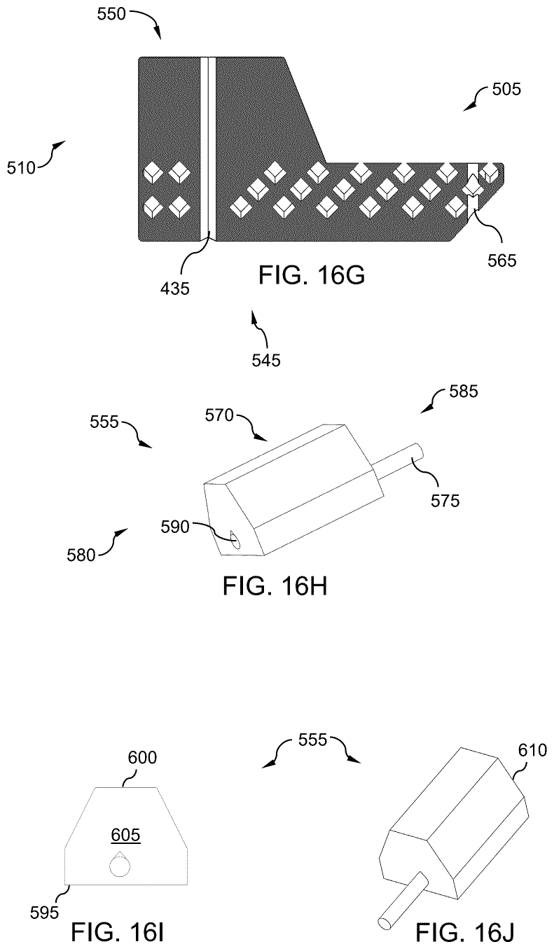

FIGS. 16H-16J illustrate different plan views of a stability block. The stability block is used to facilitate the balancing of bunion guide system on the different morphologies of a patient's foot surface. The stability block may also help to provide a more planar, balanced surface for optimized operation of the bunion guide system. Furthermore, the stability block can be used to minimize the forces that the bunion guide transmits to the anchor pins and surrounding actuators, as well as eliminating any undesired inertia forces caused by any linked actuators of the bunion guide system.

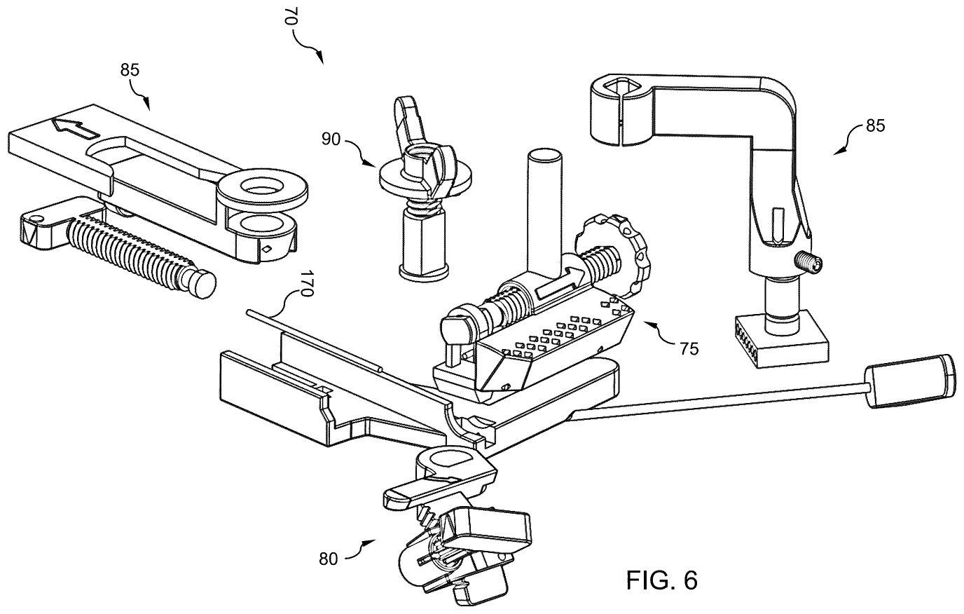

The stability block may comprise a block and a dowel pin. The block comprises a height, a width, a length, and a dowel pin opening. The dowel pin opening extends the entire length of the block. The dowel pin opening is sized and configured to receive a portion of the dowel pin. A portion of the dowel pin is disposed into the dowel pin opening of the block to secure or couple to the block. A front end of the dowel pin extends beyond a front or back surface of the block or beyond the dowel pin opening on the block. The back end of the dowel pin may be flush or inset from a front or back surface of the block. The stability block is coupled to a portion of the tarsal base as shown in FIGS. 6J-6K. The stability block is coupled to a medial side of the tarsal base. The stability block is coupled to the anchor extension body of the tarsal base. The bottom surface of the stability block may contact or engage with a skin surface of a patient. At least a bottom portion of the stability block extends below the bottom surface of the tarsal base. The length of the stability block may be optimized to help guide the physician or surgeon during fixation. The stability block may provide visual feedback during imaging and provide a trajectory of the fixation screw during the fixation step—it can be a hard stop that the surgeon collides into the stability block forcing the surgeon to adjust the trajectory. Alternatively, the stability block may be removed for more degrees of freedom during the fixation step.

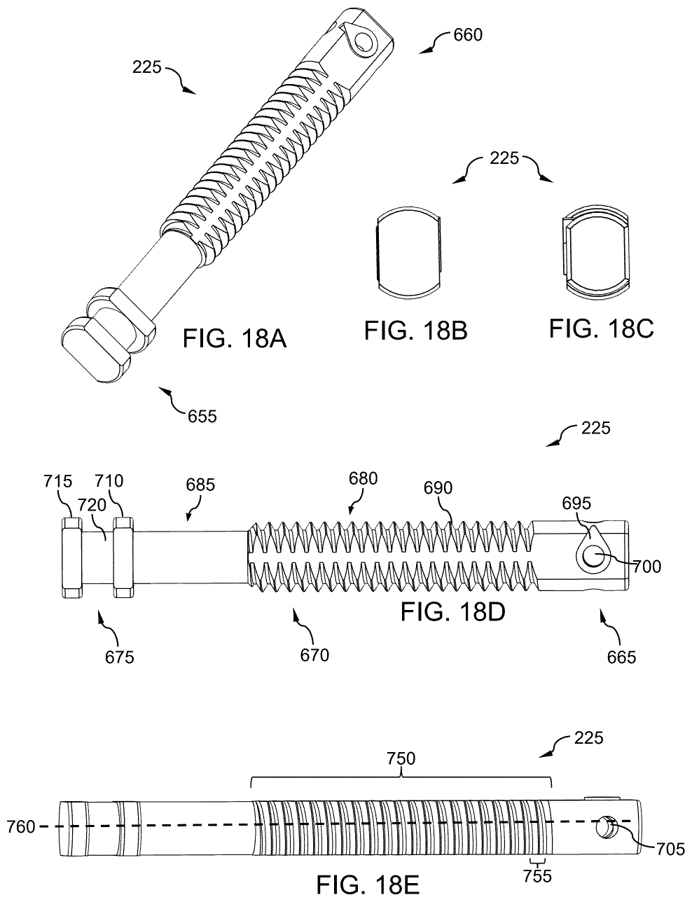

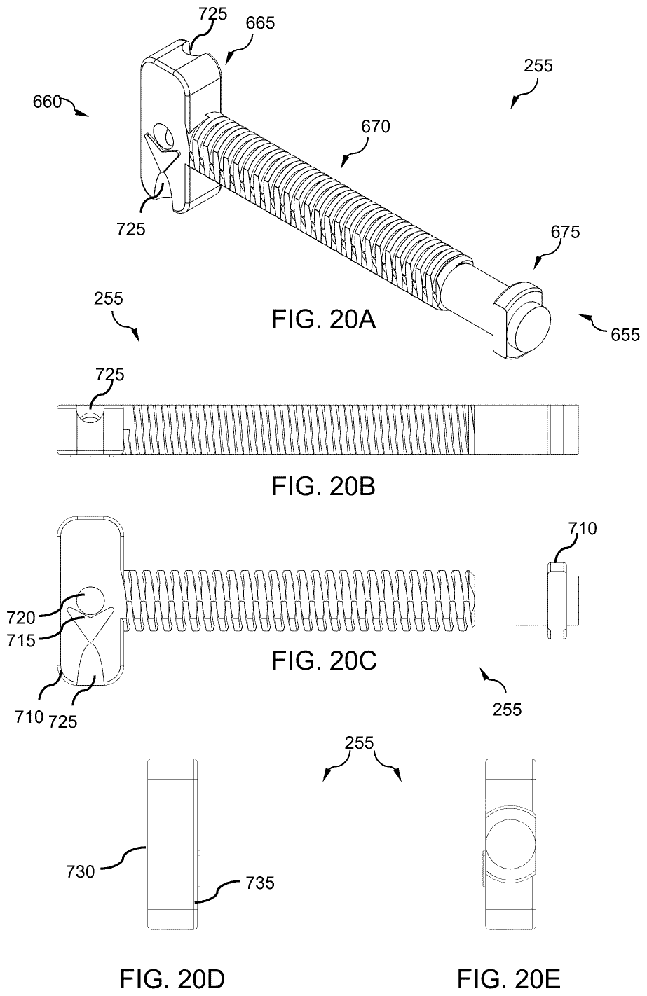

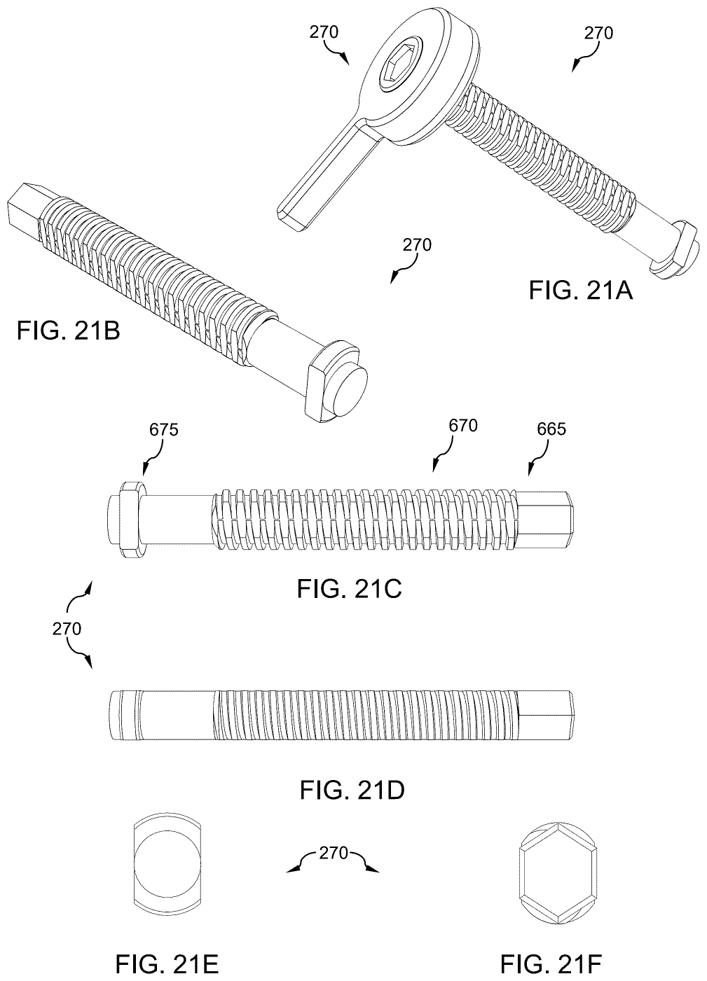

FIGS. 18A-18E, 19A-19D, 20A-20E and 21A-21F illustrates different plan views of different embodiments of a distraction screw 225, 240, 255, 270 of the first actuation mechanism 10, 75, 105, 190. Once the tarsal base of the first actuation mechanism is fixed or anchored onto the body of the first metatarsal and the rotational block of the second actuation mechanism is fixed to the capital fragment or head of the first metatarsal, the rotation of the distraction screw moves the distraction screw linearly relative to the tarsal base to move, urge, pull or slides the second portion of the angled base plate relative to the tarsal base. Such rotational-to-linear movement of the angled base plate maneuvers or manipulates the body of the metatarsal and/or the capital fragment along with the skin, soft tissues, the metaphalangeal joint and the phalanges along the longitudinal axis of the body of the first metatarsal. The distraction screw comprises a head, a shaft body and a tail. The distraction screw further comprises a first end and a second end.

The head is disposed at the second end of the distraction screw. The head may comprise a cross-sectional shape. The cross-sectional shape may include a circular, an oval, and/or a polygonal shape. The polygonal shape may include triangle, a square, a rectangle, a pentagon, and/or a hexagon. In one exemplary embodiment, the cross-sectional shape of the head rectangular and/or rounded rectangular shape. In another exemplary embodiment, the cross-sectional shape may comprise a hexagon.

The head may comprise one or more openings. The one or more openings may be sized and configured to receive a wire or pin. The wire or pin maybe inserted through the one or more openings to help the physician leverage additional force while rotating the distraction screw. The head may further comprise one or more indicators. The one or more indicators may be used to help direct the physician on the rotational direction of the distraction screw.

At least a portion of the head may be sized and configured to be disposed within a knob, wheel or ratchet wrench to provide additional support for rotational forces. At least a portion of the head may be sized and configured to be disposed within an opening of the knob, wheel or ratchet wrench. The knob, wheel and/or ratchet wrench may be coupled to a portion of the head of the distraction screw.

The shaft body may comprise a first portion and a second portion. The shaft body is disposed between the head and the tail of the distraction screw. The first portion of the shaft body may comprise threads. The threads are disposed onto the shaft body and are positioned between the head and the tail of the distraction screw. The threads comprise a major diameter, a minor diameter, a pitch, a thread angle, thread length and a helix angle. The pitch of the threads may comprise a low pitch or a high pitch. The smaller the pitch, the higher resolution, the slower adjustment speed and/or lower translation distance. The thread length equals the total translation or travel distance. The thread length or travel distance may be equal to greater than 2 mm. The thread length may be equal to or less than 6 mm. The threads may further comprise the travel distance per rotation. The travel distance per rotation of the screw may comprise 0.25 mm or greater per rotation.

The tail is disposed at the first end of the distraction screw. In one embodiment, the tail comprises a first flange, a flange channel and a second flange. The flange channel is disposed between the first flange and the second flange. The tail of the distraction screw is sized and configured to be disposed into a recess of the angled base plate and/or the recess of the second portion of the angled base plate. The flange channel is sized and configured to receive a portion of the retention element. The retention element may comprise a “U” clip.

In another embodiment, the tail comprises at least one flange and a disc. The disc is disposed onto the flange. The disc is sized and configured to be disposed within the recess of the angled base plate and/or the recess of the second portion of the angled base plate. The second portion of the shaft body is sized and configured to receive a portion of the retention element. The retention element may comprise a “U” clip. The retention element may contact or abut against a portion of the flange surface.

The tail of the distraction screw comprises an indicator and an opening. The tail is disposed at the second end. The opening is sized and configured to receive a portion of a drill wire if necessary. The indicator on the tail of the distraction screw may comprise a directionality indicator to inform the physician the clockwise rotation of the screw. The indicator may further comprise a color indicator to help the physician differentiate between the different actuation mechanisms.

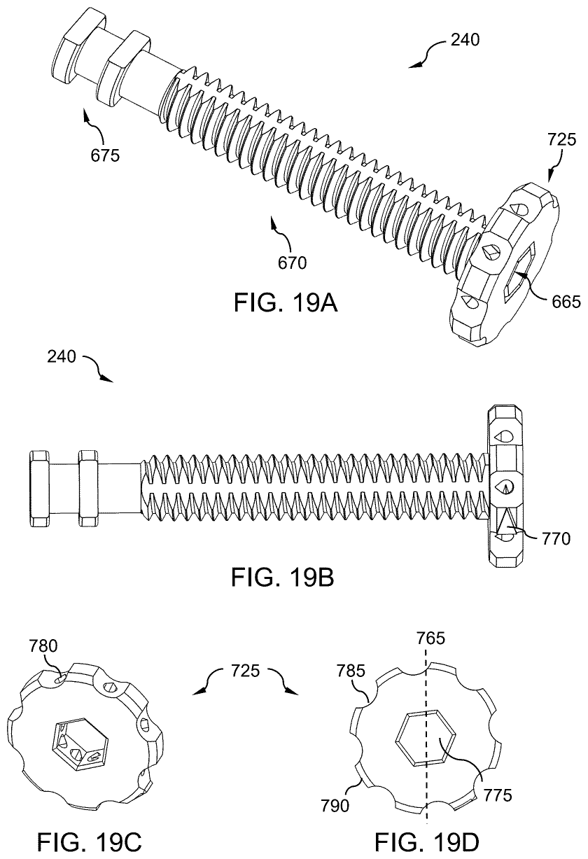

FIGS. 19C-19D illustrate different views of one embodiment of a distraction screw wheel or knob. A distraction screw wheel may be removably coupled to the second end of the distraction screw. The distraction screw wheel or knob may comprise a body and a tail opening. The body of the distraction screw wheel may comprise scalloped edges, knurling, or teeth that surround the periphery of the body to provide a more ergonomic design. The distraction screw knob can provide greater torque while turning or threading of the distraction screw.

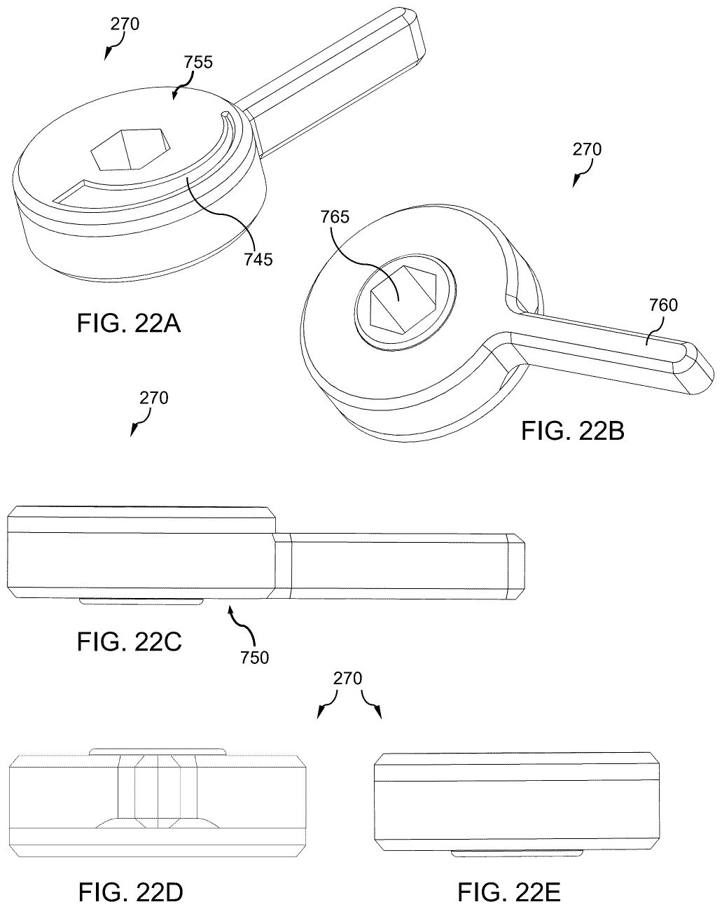

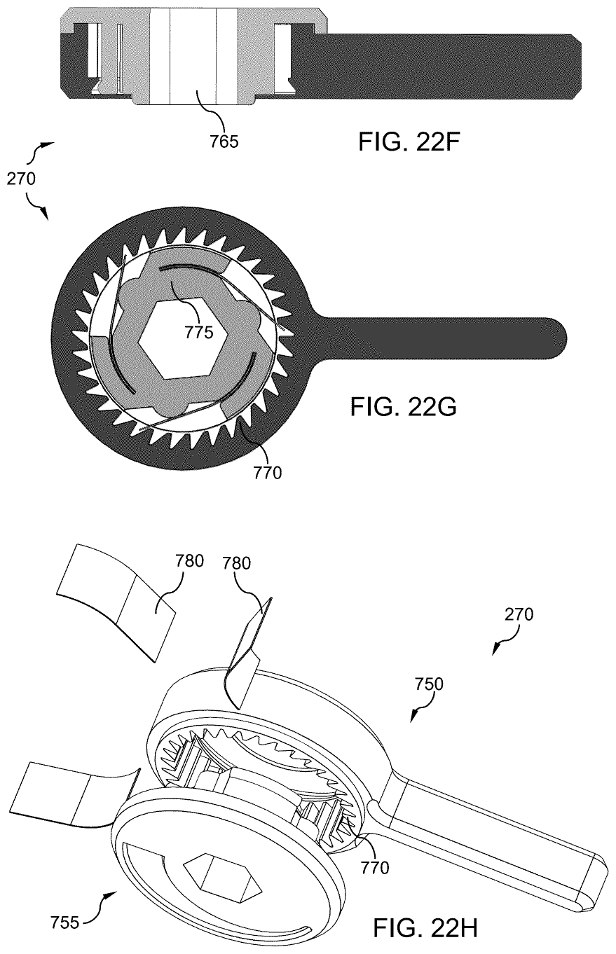

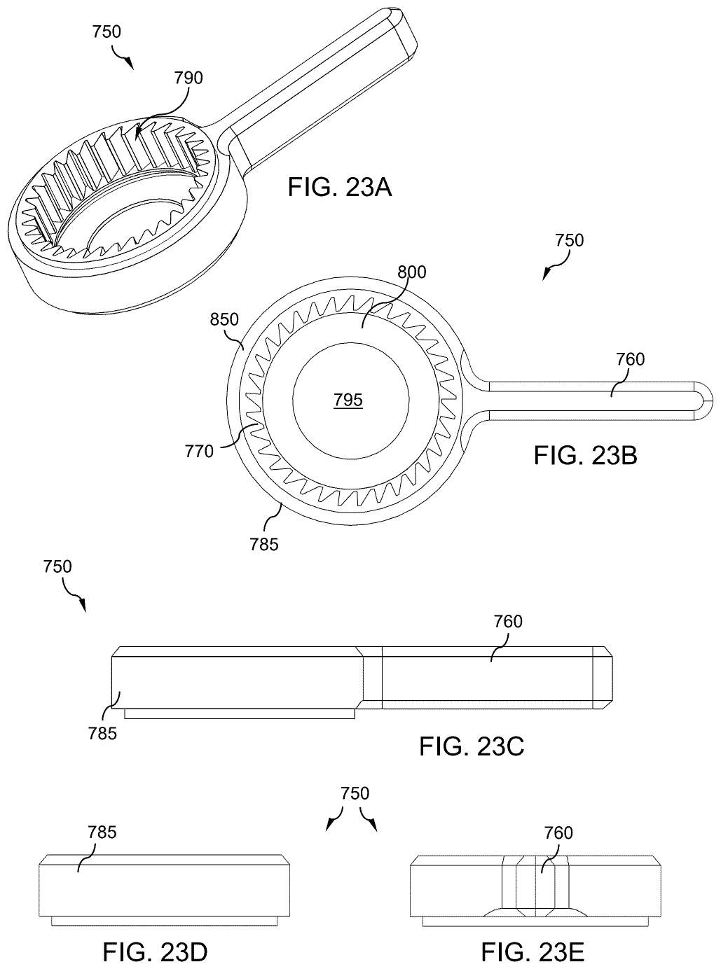

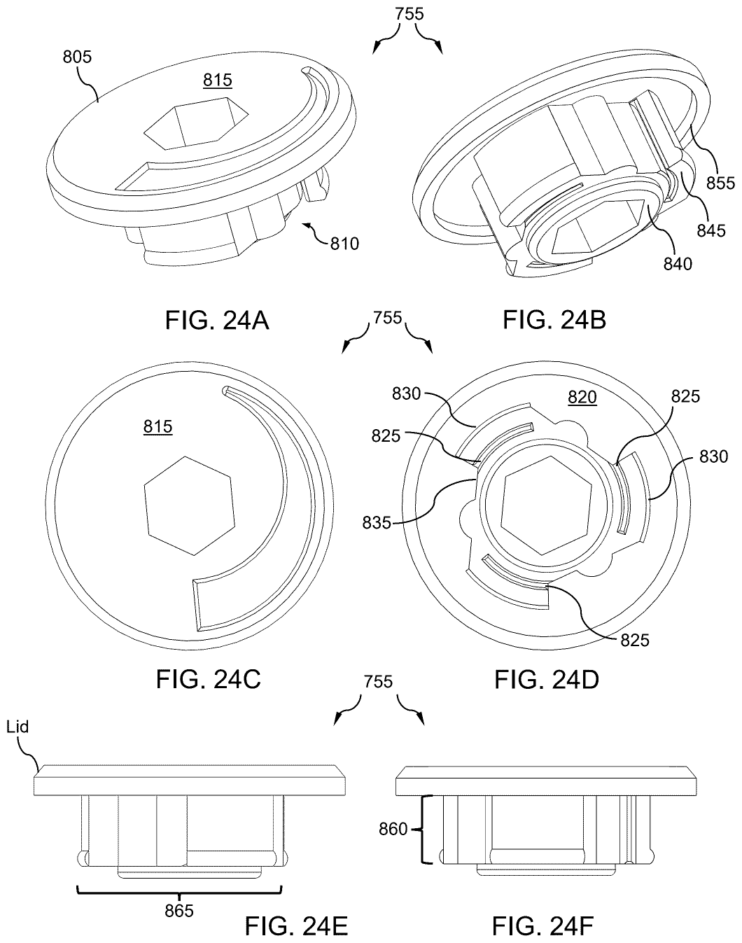

FIGS. 22A-22G, 22H, 23A-23E and 24A-24F depict various views of one embodiment of a distraction screw ratchet wrench. The distraction screw ratchet wrench comprises a ratchet arm, a drive gear base and a plurality of leaf springs. The distraction screw ratchet is designed to move only in one direction (either clockwise or counterclockwise). The drive gear base comprises a plurality of leaf springs, which the leaf springs of a spring-loaded, lever that engages with the gear teeth disposed within the ratchet arm. The gear's teeth are asymmetrically shaped (each coming to a point, with one edge being shorter and steeper than the other), which allows them to move past the leaf springs only when the drive gear base rotates in the desired direction. When the drive gear base moves in the desired direction, the gear teeth can move smoothly past the leaf springs, making a soft clicking sound. If the gear teeth begins to move in the opposite direction, the leaf springs engage with the gear teeth and locks the drive gear base in place to prevent it from moving allowing the production of torque. The distraction screw wrench is removably coupled to the second end of the distraction screw allowing the surgeon to have increased torque while rotating the distraction screw of the first actuator mechanism while distracting the head of the first metatarsal from the body of the first metatarsal.

FIGS. 25-27 illustrate isometric views of different embodiments of the second actuation mechanism. The second actuation mechanism may enable or allow the rotation of the capital fragment around the longitudinal axis of the first metatarsal. The second actuation mechanism allows or enables the physician to rotate the gear knob (i.e., pinion gear) so that the gear knob engages with the teeth on the arc gear rack, which causes the arch gear rack to move relative to the gear knob and the rotation block. Since the arc gear rack comprises an arcuate shape, the arc gear rack moves along a segment of a circle or an arc length, the arc length having a measurement in degrees. The second actuation mechanism comprises a rotation block, an arc gear knob and an arc gear rack.

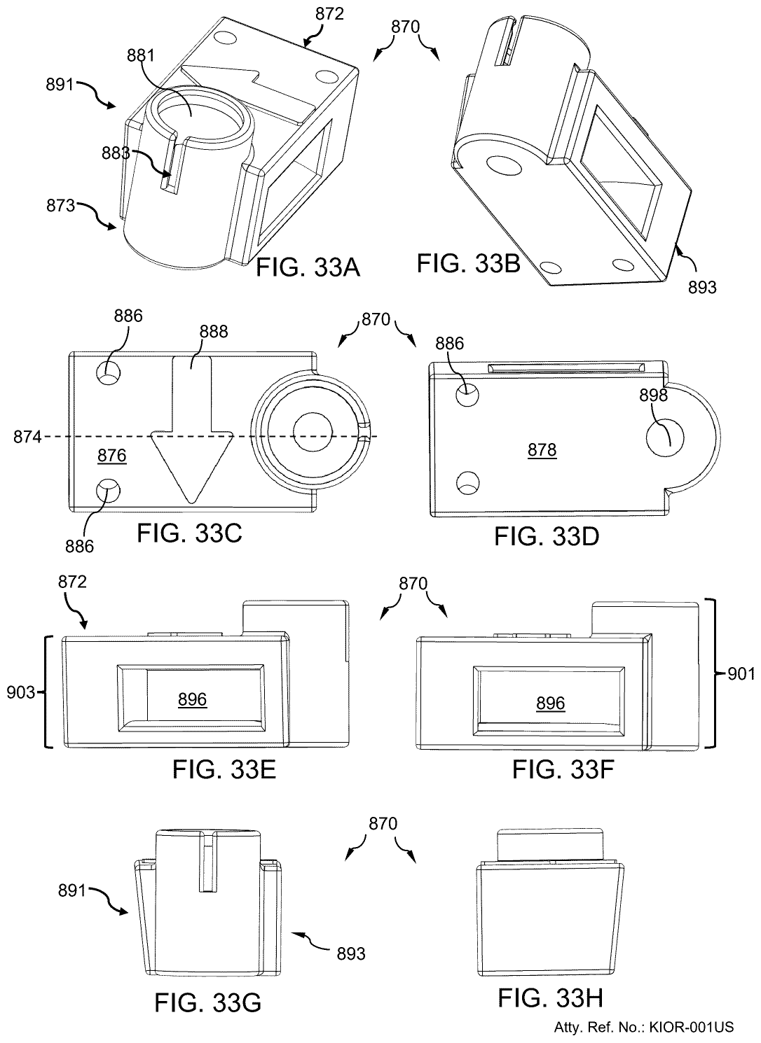

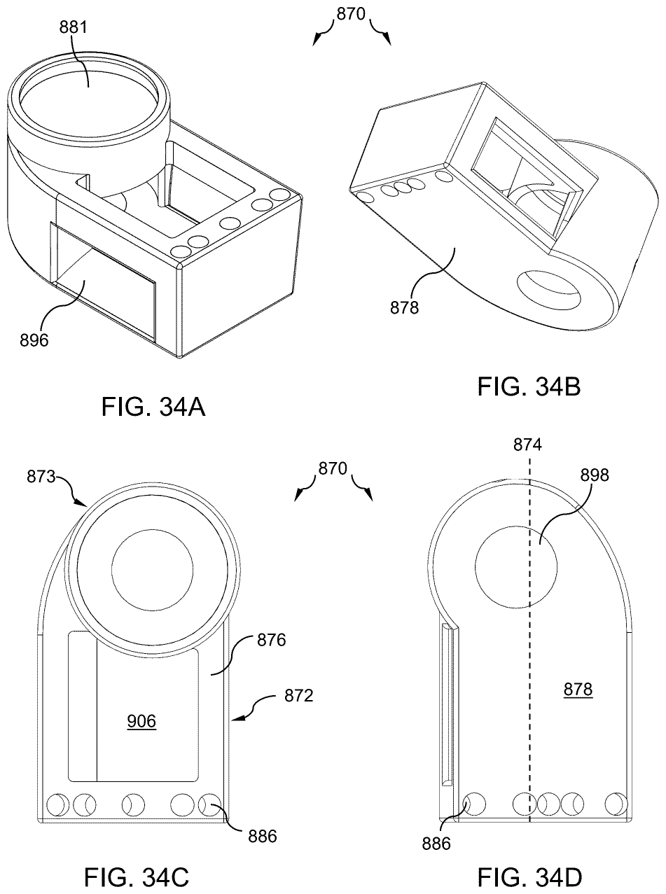

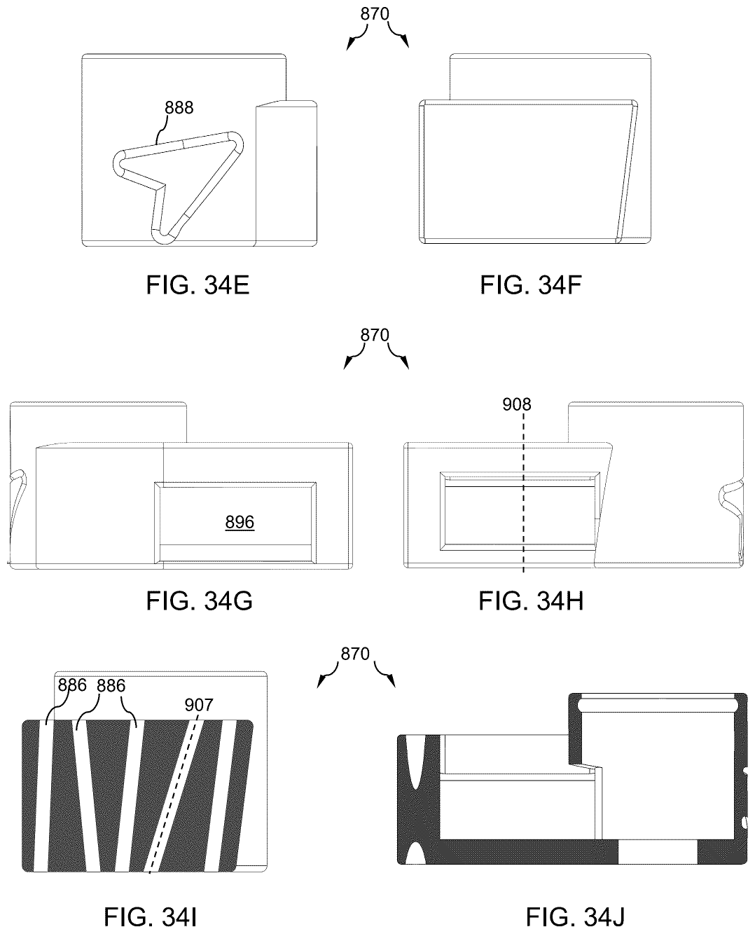

FIGS. 33A-33H and 34A-34J illustrate various plan views of different embodiments of a rotation block of the second actuation mechanism. The rotation block comprises one or more openings, a cylinder or bore and a block housing. The block housing comprises a passageway or window and one or more openings. The passageway extends from a first side through the second side. The passageway is disposed transverse to the longitudinal axis of the rotational block. The passageway includes a length and width. The passageway being sized and configured to receive a portion of the rotational arc rack. The passageway being sized and configured to receive a portion of the arc rack segment.

The cylinder comprises a bore. The bore is sized and configured to receive a portion of the rotational gear knob. The bore extends from a top surface of the cylinder towards the bottom surface. The cylinder extends from a bottom surface of the block housing upwardly and away from the top surface of the block housing. The cylinder comprising a cylinder height, the cylinder height being larger than the than a block housing height.

The block housing comprises a longitudinal axis first passageway or window, a second passageway or window, and one or more openings. The first passageway extends from a first side through the second side of the block housing. The first passageway is disposed transverse to the longitudinal axis of the rotational block. The first passageway includes a first length and first width. The first passageway being sized and configured to receive a portion of the rotational arc rack. The passageway being sized and configured to receive a portion of the arc rack segment. The second passageway extends from a top surface towards the bottom surface. The second passageway allows the viewing of the directional indicator for the second actuator mechanism. The second passageway follows the axial axis of the rotation block or the block housing.

The cylinder or bore housing comprises a bore. The bore is sized and configured to receive a portion of the rotational gear knob. The bore extends from a top surface of the cylinder or bore housing towards the bottom surface. The cylinder or bore housing extends from a bottom surface of the block housing upwardly and away from the top surface of the block housing. The cylinder or bore housing comprising a cylinder height, the cylinder height being larger than the than a block housing height.

The rotation block may further comprise one or more openings. The one or more openings may be sized and configured to receive a portion of anchoring pins or wires. Each of the one or more openings comprise a opening diameter and/or a longitudinal axis. Each of the longitudinal axis of the one or more openings may be oriented at an angle. Each of the longitudinal axis of the one or more openings may be angled relative to the axial axis of the rotation block. Alternatively, each of the longitudinal of the one or more openings may be orthogonal to the longitudinal axis of the rotation block and/or follow or be parallel to the axial axis of the rotation block. Each of the orientation angles of the longitudinal axis of the one or more openings of the rotation block are the same. Each of the orientation angles of the longitudinal axis of the one or more openings of the rotation block are different. At least one of the orientation angles of the longitudinal axis of the one or more openings of the rotation block are the same. At least one of the orientation angles of the longitudinal axis of the one or more openings of the rotation block are different.

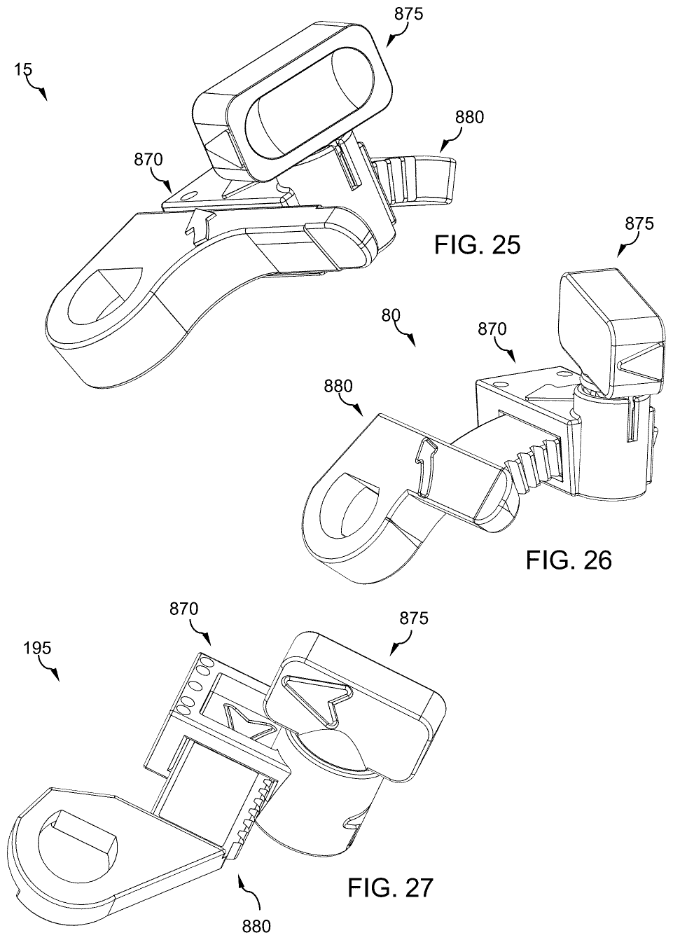

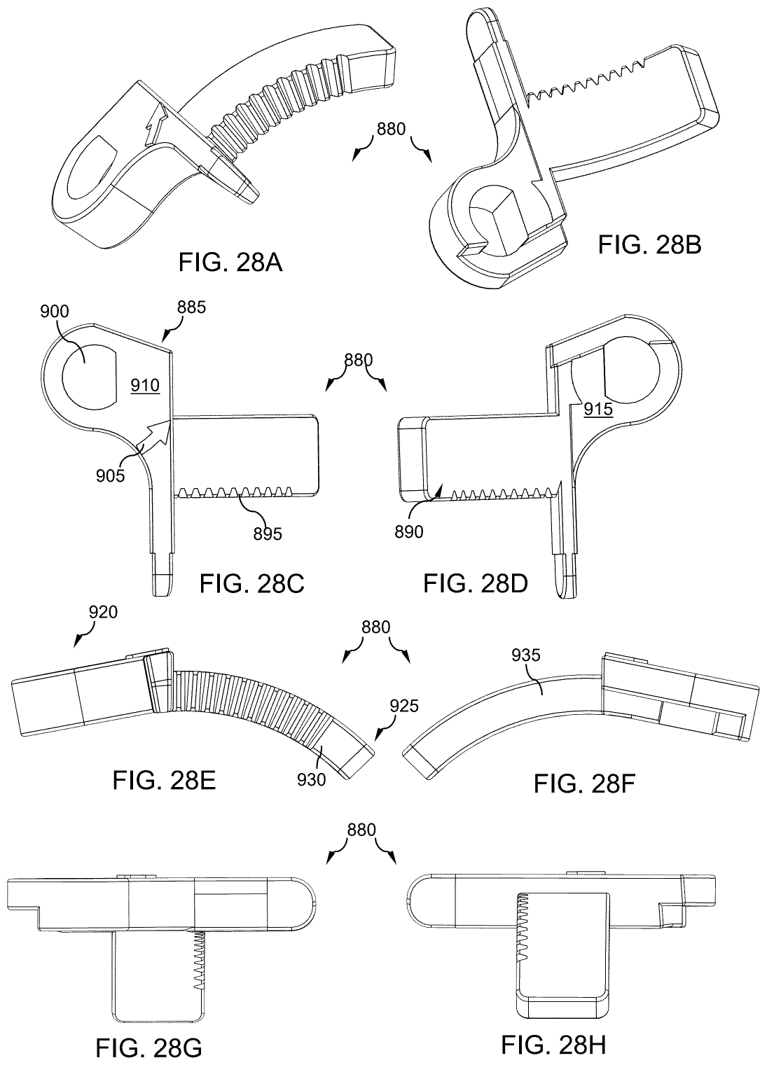

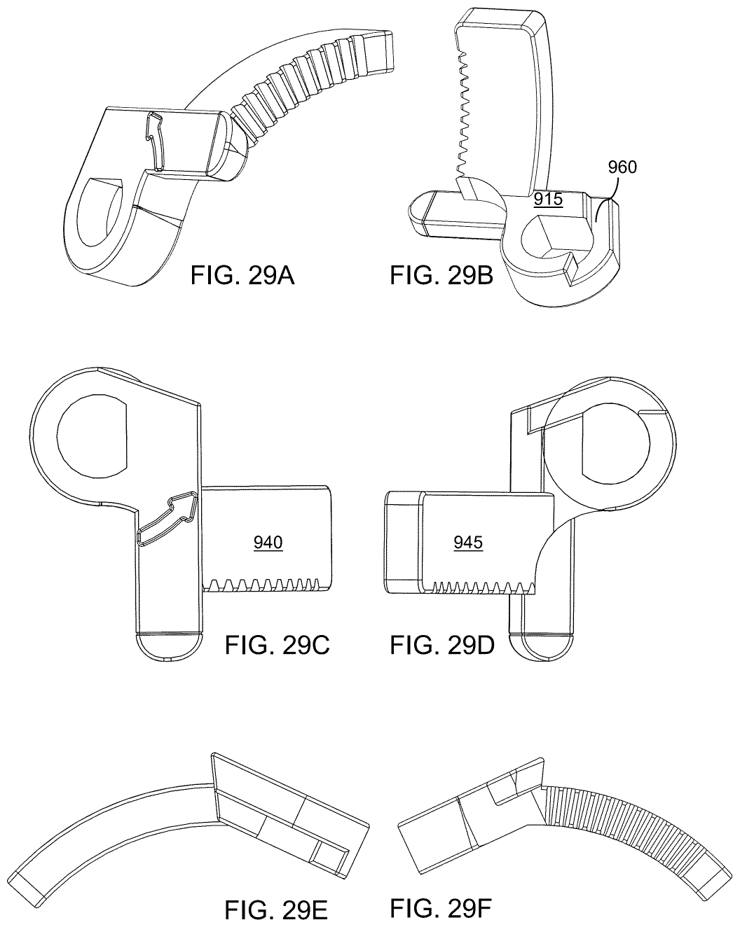

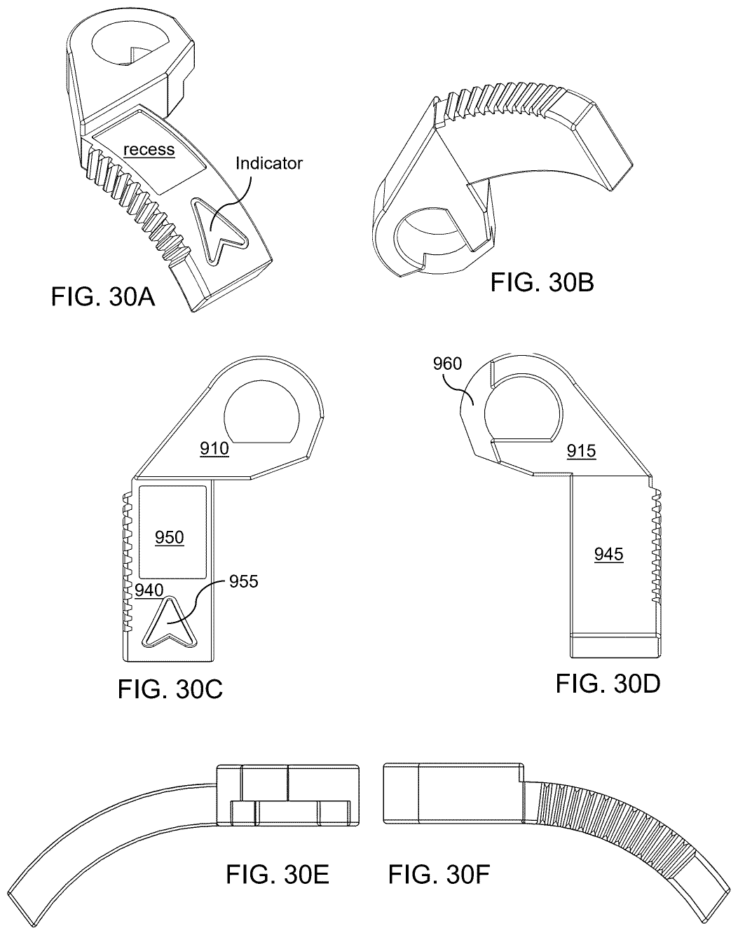

FIGS. 28A-28H, 29A-29F and 30A-30F and illustrate various plan views of different embodiments of the rotation gear rack of the second actuation mechanism. The rotation gear rack comprises a rotation base and the arc segment. The base comprises an opening, an indicator and/or a shape. At least a portion of the base is sized and configured to be disposed within the screw housing channel of the displacement screw housing of the 3rd actuation mechanism. Accordingly, at least a portion of the base engages with the screw housing to allow rotation of the 4th actuation mechanism relative to the 2nd actuation mechanism after the 2nd actuation mechanism is fixed to the capital fragment and adjoining phalanges. The shape of the base comprises a “d” or “p” shape and/or substantially similar “d” or “p” shape. The opening is sized and configured to receive a portion of the rotational gear knob.

The arc segment is coupled to the rotation base. A first end of the arc segment is coupled to the rotation base. The arc segment extends from a surface of the rotation base. The arc segment further comprises a top surface, a bottom surface, a first side, a second side and arc length and teeth. The arc teeth are disposed onto a first or second side surface. The arc length is a fraction of the circumference of a circle, and it is a measure given in degrees. The arc length may comprise 0 degrees to 45 degrees; 0 degrees to 30 degrees; and/or 0 degrees to 15 degrees. The arc segment may further comprise at least one indicator.

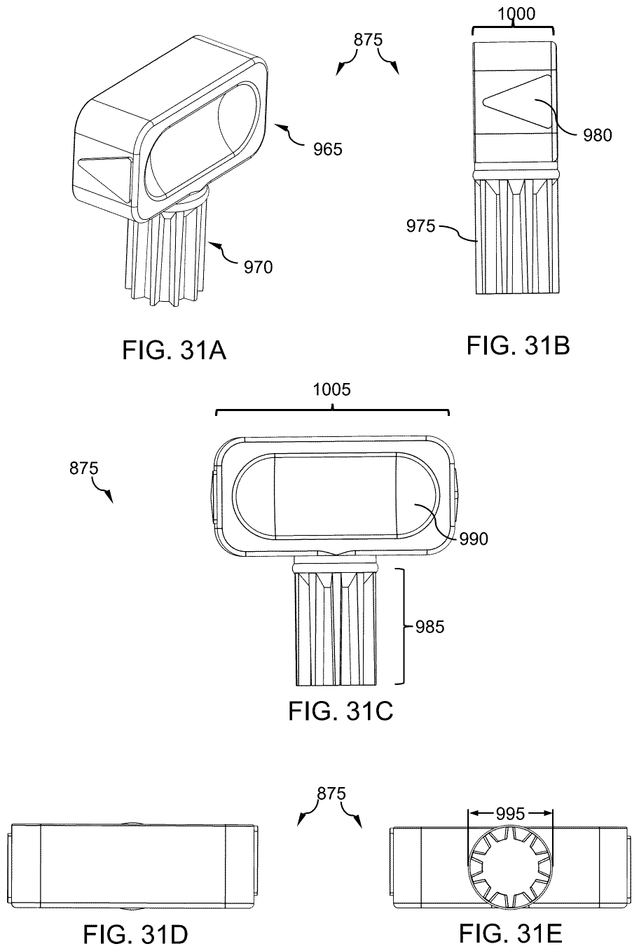

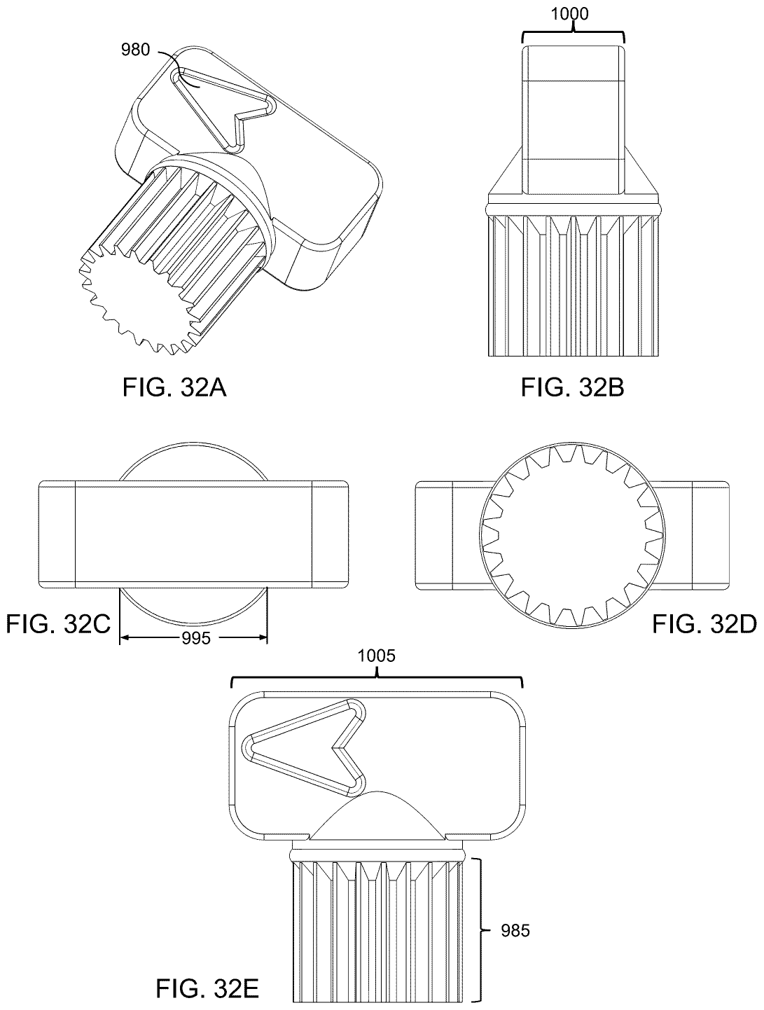

FIGS. 31A-31E and 32A-32E illustrate various plan views of different embodiments of a rotation gear knob of the second actuation mechanism. The rotation gear knob comprises a head and a gear. The head comprises a top surface, a bottom surface, a first side surface, and/or a second side surface. The head further comprises an indicator and a shape. The shape of the head may comprise a circle, an oval, and/or a polygon. The polygon shape may include a triangle, a rectangle, a square, pentagon or hexagon. In one exemplary embodiment, the head comprises a polygon shape, the polygon shape is a rectangle. The head may further comprise filleted or rounded corners or edges. The head may further comprise a recess or depression. The recess extends from a first or second side surface toward the first or second side surface. The recess may be sized and configured to receive a portion of an average physician's thumb or finger.

The gear comprises an outer diameter, a height, and/or a plurality of teeth. The plurality of teeth engages with the arc segment teeth to enable the movement or rotation of the capital segment (and also along with the adjoining phalanges and metaphalangeal joints) around the longitudinal axis of the body of the first metatarsal. The gear further comprises a rotational ratio, the rotational ratio equals one rotation for the total arc length. Therefore, a one-quarter turn of the rotation gear knob may equal one-quarter of total arc length of the arc segment.

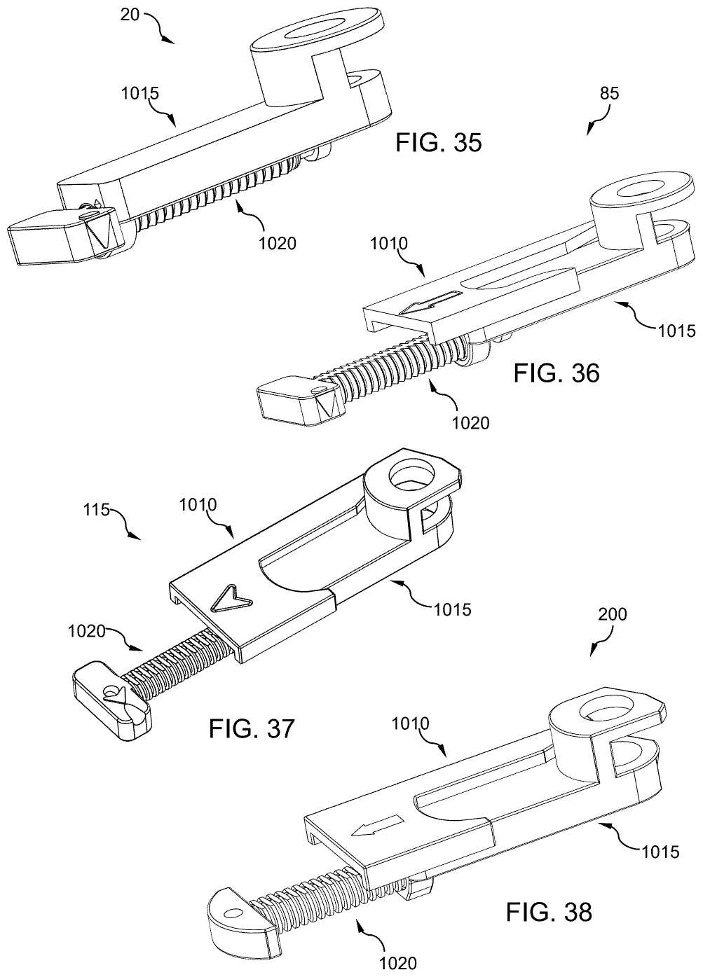

FIGS. 35-38 illustrate various views of different embodiments of a third actuation mechanism 20, 85, 115, 200. The trajectory placement is transverse and/or substantially transverse to the longitudinal axis of the metatarsal body. The third actuator mechanism is also a mechanical linear actuator that converts rotational motion into linear motion. Its operation relies on the rotation of the translation screw to move the translation housing or slide to the angled base plate. The translation slide or housing engages with the second and fourth actuator mechanism allowing the movement of the head of the first metatarsal from medial to lateral direction. Alternatively, the movement of the head of the first metatarsal displaces in the direction towards the second metatarsal. In one embodiment of the third actuator mechanism, the actuator mechanism facilitates translation of the capital fragment to move, translate or displace from the longitudinal axis of the first metatarsal body toward the second metatarsal or toward the trajectory placement from lateral to medial direction.

Furthermore, once the tarsal base of the first actuation mechanism is fixed onto the body of the first metatarsal and the rotational block of the second actuation mechanism is fixed to the capital fragment or head of the first metatarsal, the rotation of the translation screw moves the translation housing linearly to move, urge, pull or slide head of the first metatarsal (e.g., capital fragment) from the medial to lateral direction. Since the third actuation mechanism engages with the second actuation mechanism and the fourth actuator mechanism, all three mechanisms also move relative to the angled base plate. Such rotational-to-linear movement of the translational screw of the third actuation mechanism maneuvers or manipulates the distracted and rotated capital fragment (may also move with the skin, soft tissues), the metaphalangeal joint and/or the phalanges along the planned trajectory toward the second metatarsal. The third actuation mechanism comprises a translation screw and a translation screw housing, and translation slide cover.

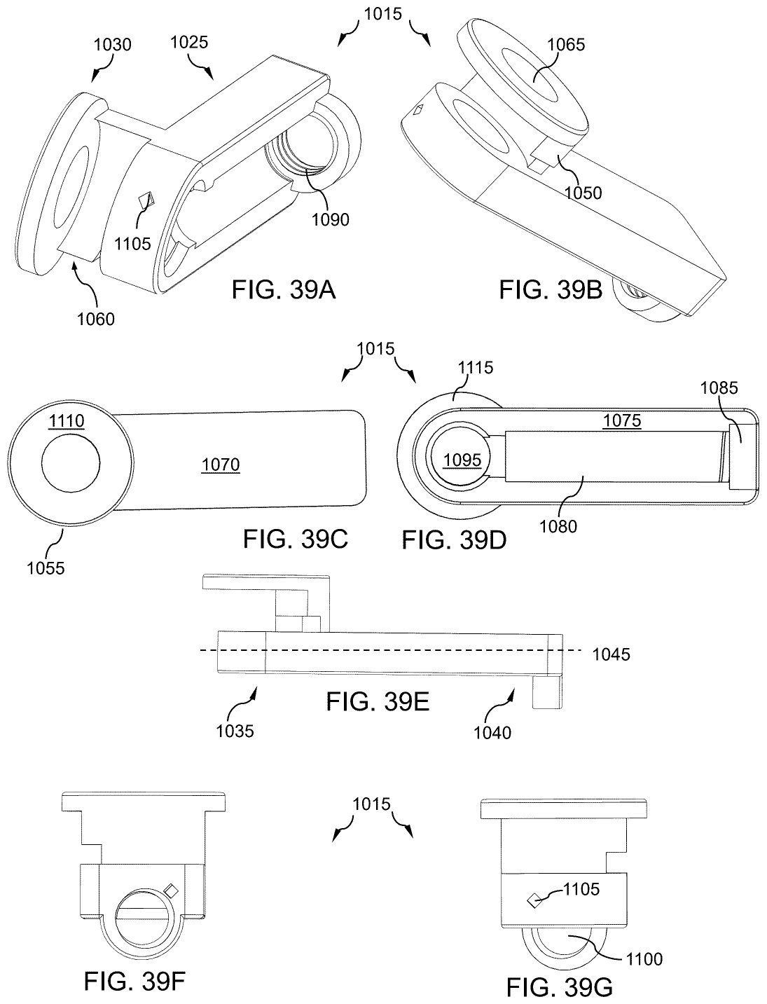

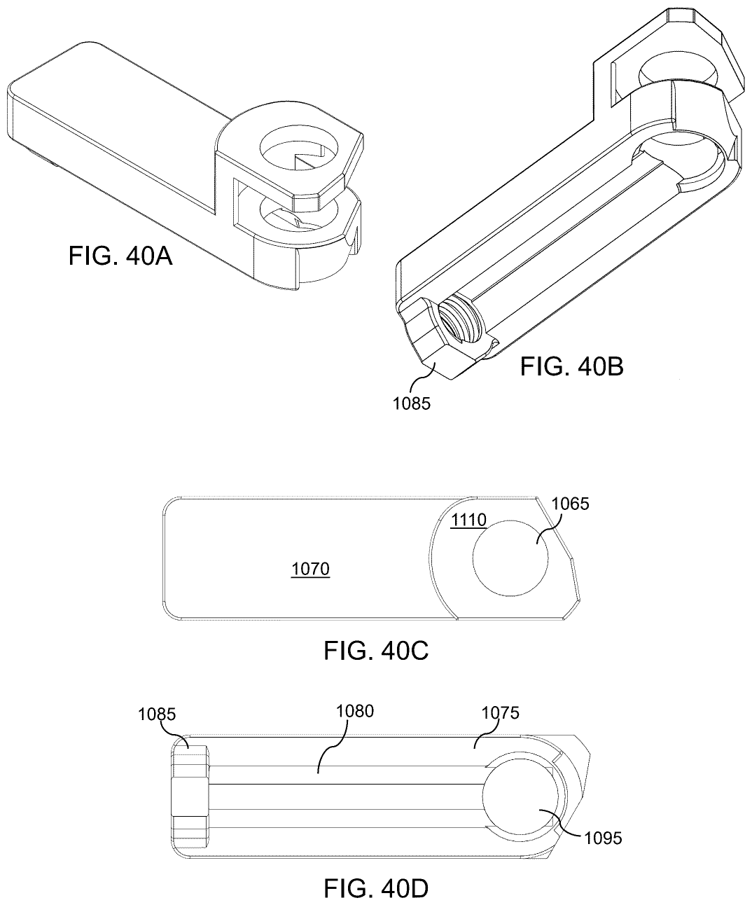

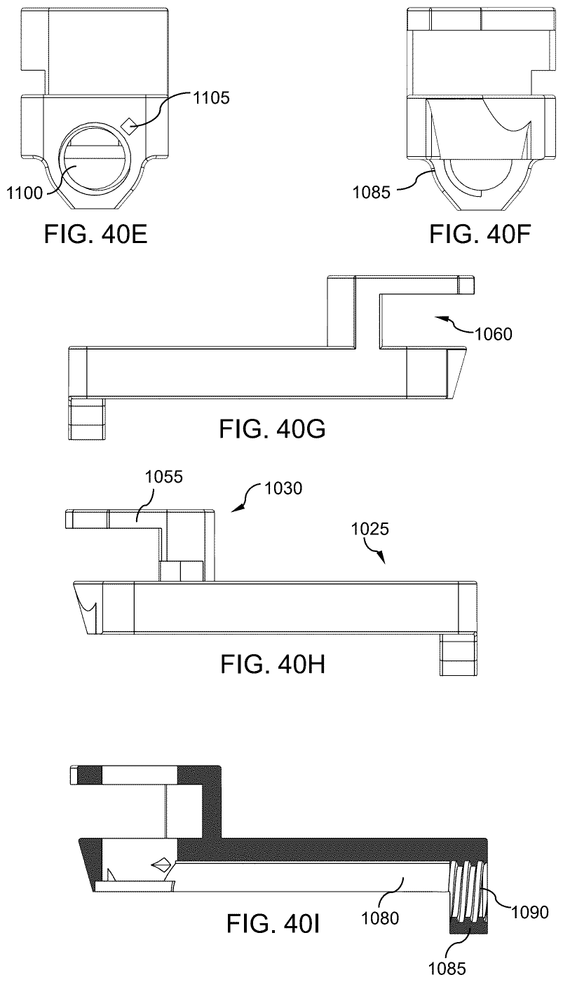

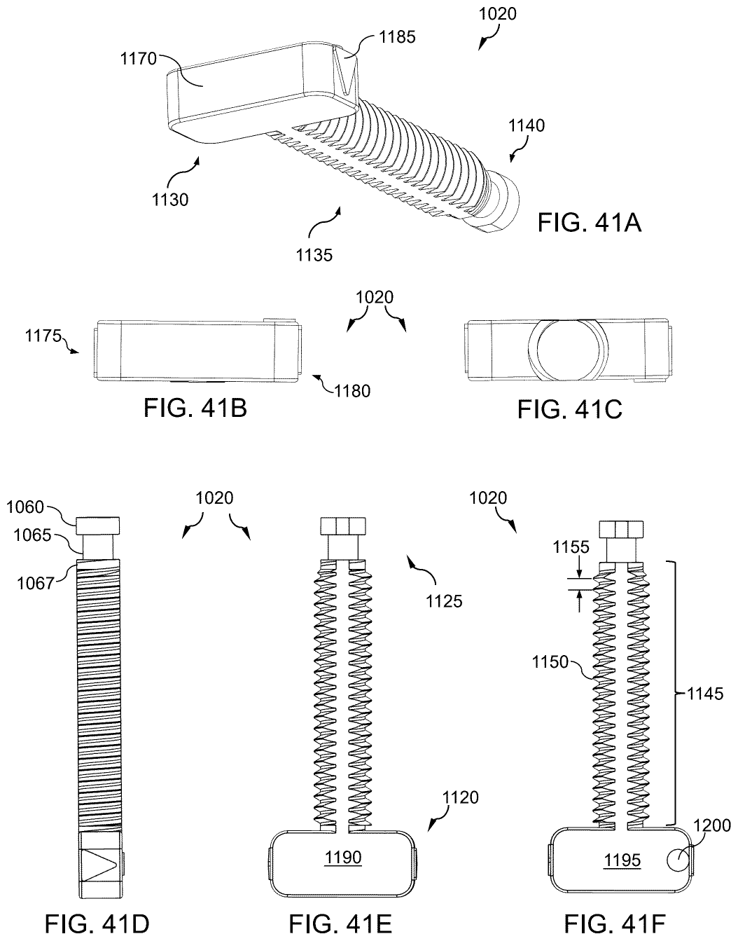

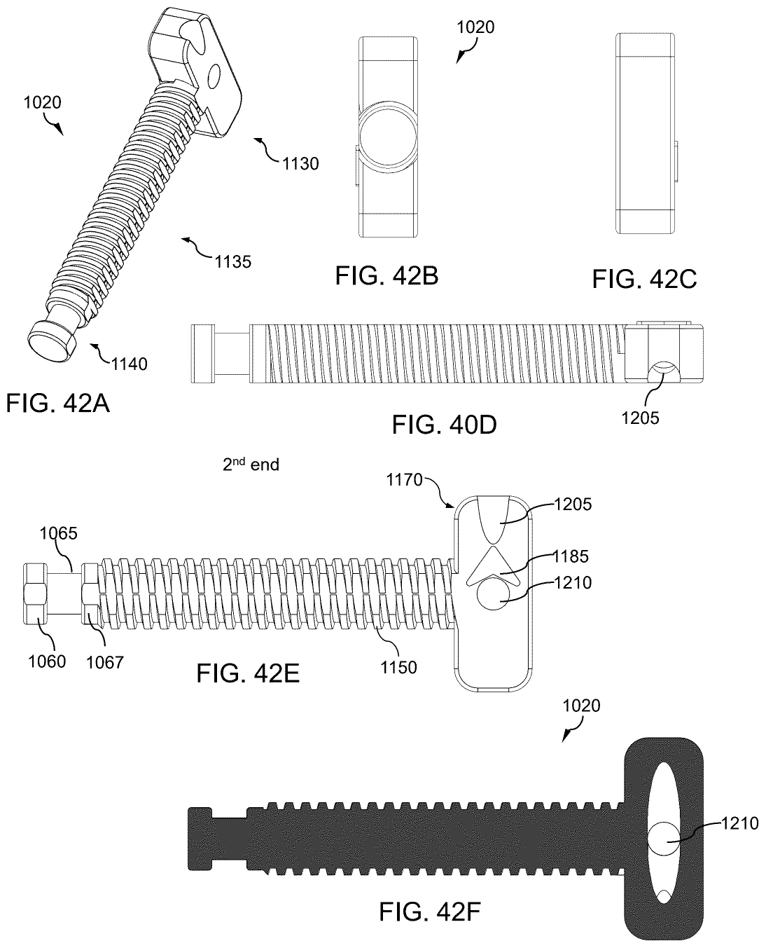

FIGS. 39A-39G and 40A-40I illustrates several plan views of different embodiments of a translation or displacement screw housing 1015 of the third actuator mechanism 20, 85, 115, 200. The translation or displacement screw housing comprises a longitudinal axis, a translation screw base or screw platform, and a translation screw cap or housing canopy, and a screw wall or ring. The translation screw base or screw platform comprises a first end, a second end, a top surface, a bottom surface, a first opening and a second opening. The bottom surface of the screw base comprises a recess. The recess extends from a second end of the translation screw platform or base towards the first end of the translation screw platform or base. The recess includes a recess length, the recess length is shorter than a translation screw base or platform length. The recess of the screw base is sized and configured to receive a portion of the translation or displacement screw of the third actuation mechanism. At least a portion of the recess may comprise threads. At least a portion of the recess at the second end comprises threads. At least a portion of the ring wall or screw wall extends from a bottom surface of the translation screw base or platform towards the inferior direction. The ring wall or screw wall comprises an opening, the opening includes threads. The threaded opening is sized and configured to receive a portion of the translation screw.

The first through-hole or opening is disposed adjacent to the first end of the translation screw base or platform. The first through-hole or opening extends from a bottom surface of the screw base through the top surface of the translation screw base or platform. The first through-hole or opening is sized and configured to receive a portion of the PASA pin of the fourth actuation mechanism. A first opening axis is transverse to the longitudinal axis of the translation screw base or platform. The second through-hole or opening extends from a first end of the translation screw base or platform through the second end of the translation screw base or platform. The second through-hole or opening axis extends parallel to the longitudinal axis of the translation screw base or platform.

The translation screw canopy comprises a translation screw wall and a cap. The canopy is disposed at or adjacent to the first end of the translation screw housing or platform. The translation screw wall extends upwardly from a top surface of the screw base or platform. The translation screw wall extends perpendicular to the top surface of the translation screw base. At least a portion of the translation screw cap is disposed onto a top surface of the screw wall. At least a portion of the translation screw cap extends horizontally from the translation screw wall. The translation screw cap is spaced apart from the top surface of the screw base to create a channel or cavity. The translation screw wall is disposed between the translation screw cap and the top surface of the screw base. The translation screw cap comprises an DMAA pin opening or through-hole or a third opening. The DMAA pin opening or through-hole extends from a top surface of the screw cap to the bottom surface of the screw cap. The DMAA pin opening of the screw cap is coaxially aligned with the first opening of the translation screw base or platform. The first opening and the DMAA pin opening is sized and configured to receive a portion of the PASA or DMAA pin.

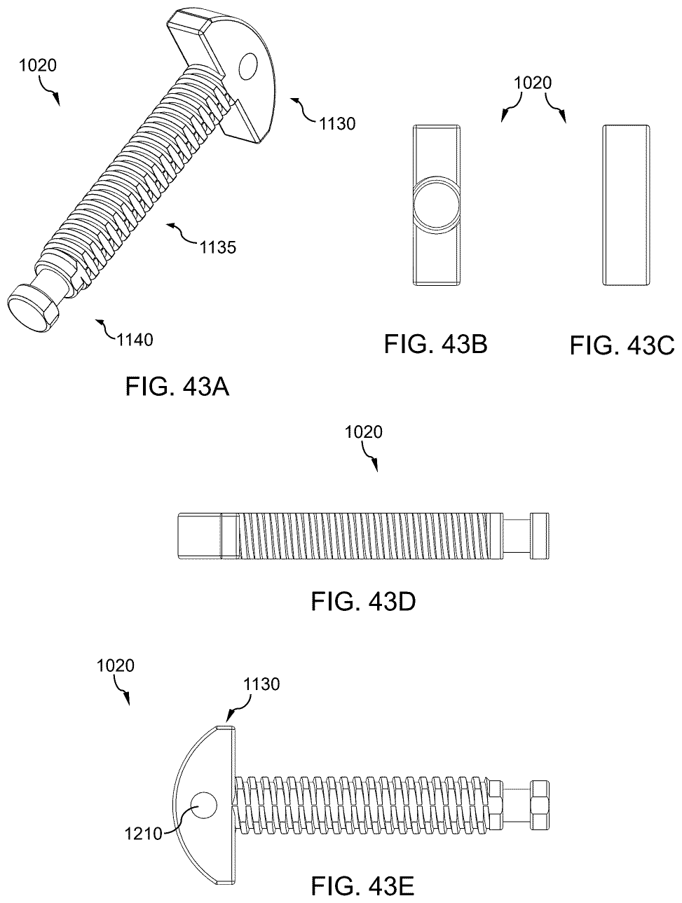

FIGS. 41A-41F, 42A-42F and 43A-43E illustrates different plan views of different embodiments of a displacement or translation screw 1020 of the third actuator mechanism 20, 85, 115, 200. The translational screw 1020 comprises a head 1130, a shaft body 1135 and a tail 1140

The head is disposed at the first end. The head comprises a top surface, a bottom surface, a first side surface, and/or a second side surface. The head further comprises an indicator and a shape. The shape of the head may comprise a circle, semi-circle an oval, and/or a polygon. The polygon shape may include a triangle, a rectangle, a square, pentagon or hexagon. In one exemplary embodiment, the head comprises a polygon shape, the polygon shape is a rectangle. In another exemplary embodiment, the head comprises a semi-circle shape. The head may further comprise filleted or rounded corners or edges. The head may further comprise a recess or depression (not shown). The recess extends from a first or second side surface toward the first or second side surface. The recess may be sized and configured to receive a portion of an average physician's thumb or finger. The head may further comprise an indicator. The indicator disposed onto a first side or second side surface. The indicator comprises a color and directionality indicator. The head of the displacement screw is adjacent to the second end of the displacement screw housing.

The shaft body is disposed between the head and the tail of the translation screw. The shaft body may comprise threads. The threads are disposed onto the shaft body and are positioned between the head and the tail of the displacement or translation screw. The threads comprise a major diameter, a minor diameter, a pitch, a thread angle, thread length and a helix angle. The pitch of the threads may comprise a low pitch or a high pitch. The smaller the pitch, the higher resolution, the slower adjustment speed and/or lower translation distance. The thread length equals the total translation or travel distance. The thread length or travel distance may be equal to or less than 50 mm. The thread length or travel distance may be equal to or less than 40 mm. The threads may further comprise the travel distance per rotation. The travel distance per rotation of the screw may comprise at least 5 mm or greater per rotation.

The tail of the translation screw is disposed at the second end. The tail comprises a flange and a shaft portion. The flange is spaced apart from a second end of the shaft body. The shaft portion between the second end of the shaft body and the flange. The tail is sized and configured to be disposed within the recess of the displacement screw housing. In another embodiment, the tail comprises a first flange, a second flange, and a shaft portion or segment. The shaft segment or portion is disposed between the first flange and the second flange. The shaft segment includes a segment diameter, the segment diameter of the shaft segment is smaller than a first or second flange diameter or width. The tail of the translation screw is disposed into the recess of the first portion of the angled base plate. The translation screw is not slidable relative to the angled base plate.

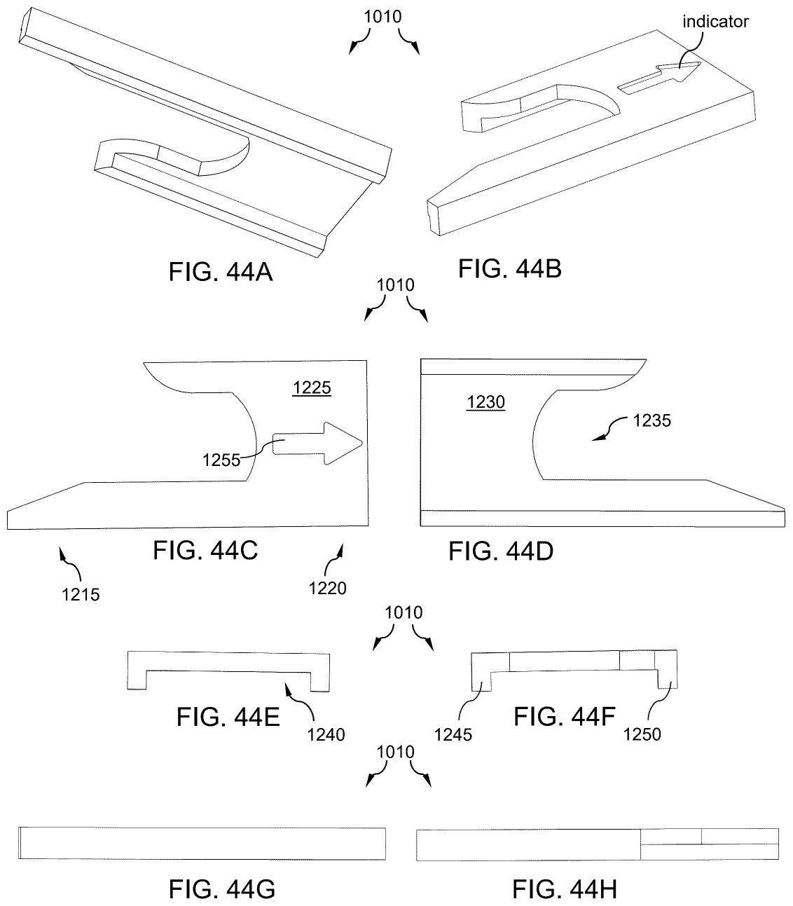

FIGS. 44A-44H illustrate various plan views of one embodiment of a top cap 1010. The top cap comprises a top surface, a bottom surface, a front surface and a back surface. The top surface comprises an indicator. The indicator comprises a directionality and/or a color indicator. The bottom surface comprises a track. The top cap track width is larger than the than the track of the second portion of the angled base plate. The top cap track is sized and configured to receive a portion of the second portion track of the angled base plate. The top cap track is disposed over a portion of the second portion track of the angled base plate. The top cap track extends from a first end to a second end.

The top cap further comprises a guide slot. The guide slot of the top cap may align or substantially align with the guide slot of the angled base plate. The guide slot comprises a slot width and a slot length. The slot width and slot length may be sized and configured to accommodate the viewing of the first end of the metatarsal that includes at least a portion of the metatarsal head (e.g., capital fragment) and the metaphalangeal joint. This viewing window comprising the slot width and slot length may guide the physician to perform the necessary osteotomy to distract the capital fragment from the body of the first metatarsal.

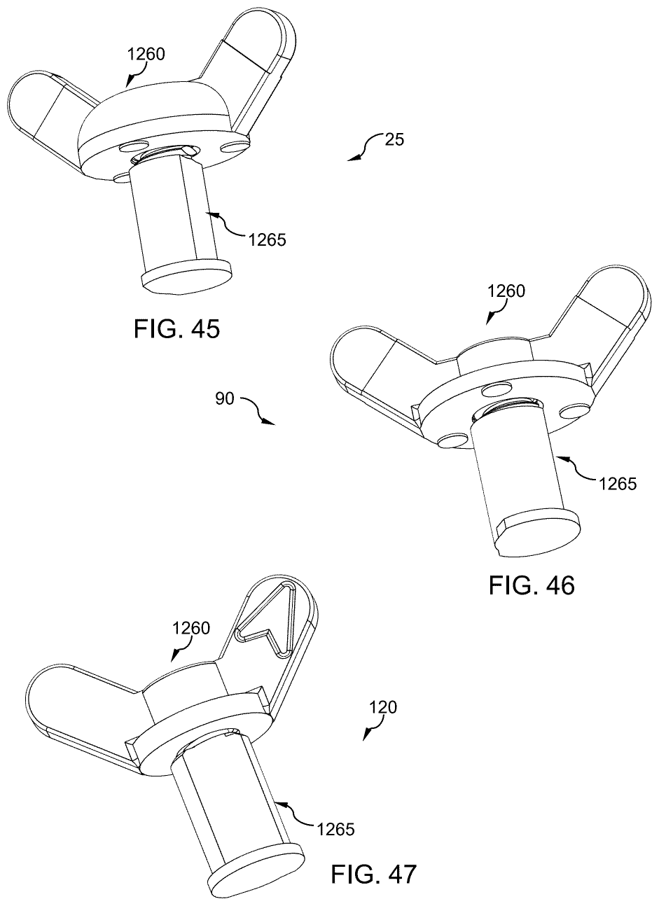

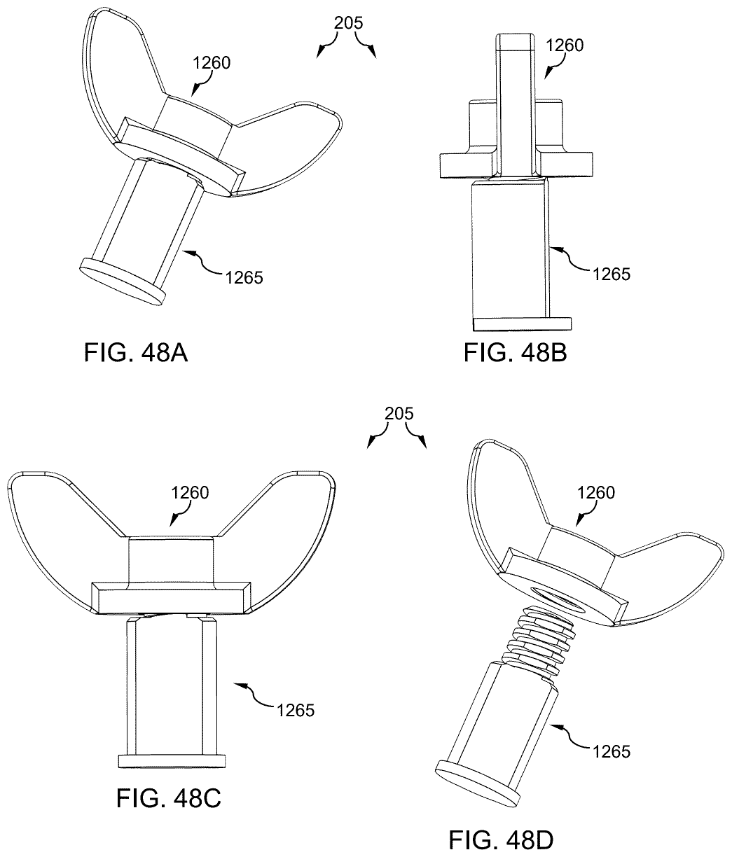

FIGS. 45-47 and 48A-48D illustrate various views of different embodiments of a fourth actuation mechanism 25, 90, 120, 205. The fourth actuation mechanism may enable or allow the inversion adduction correction of the capital fragment for the final cosmetic alignment of the Great Toe. The fourth actuation mechanism comprises a PASA or DMAA pin and a wingnut. The fourth actuation or actuator mechanism is inserted through the opening of the canopy, the opening of the platform or base of the translation housing or slide and the opening of the rotation gear rack. The opening of the rotation gear rack comprises an opening shape that matches or substantially matches the shaft body of the DMAA pin to allow the DMAA pin to engage with the rotation gear rack and prevent free movement or rotation within the opening of the rotation gear rack forcing the turning or rotation of the rotation gear rack of the third actuator mechanism that fixes the capital fragment. The fourth actuator mechanism can freely rotate within the opening of the canopy and the platform or base of the translation housing. The opening shape of the rotation gear rack comprises a “D” shape. The rotation of the wingnut may comprise clockwise or counterclockwise rotation. The flat

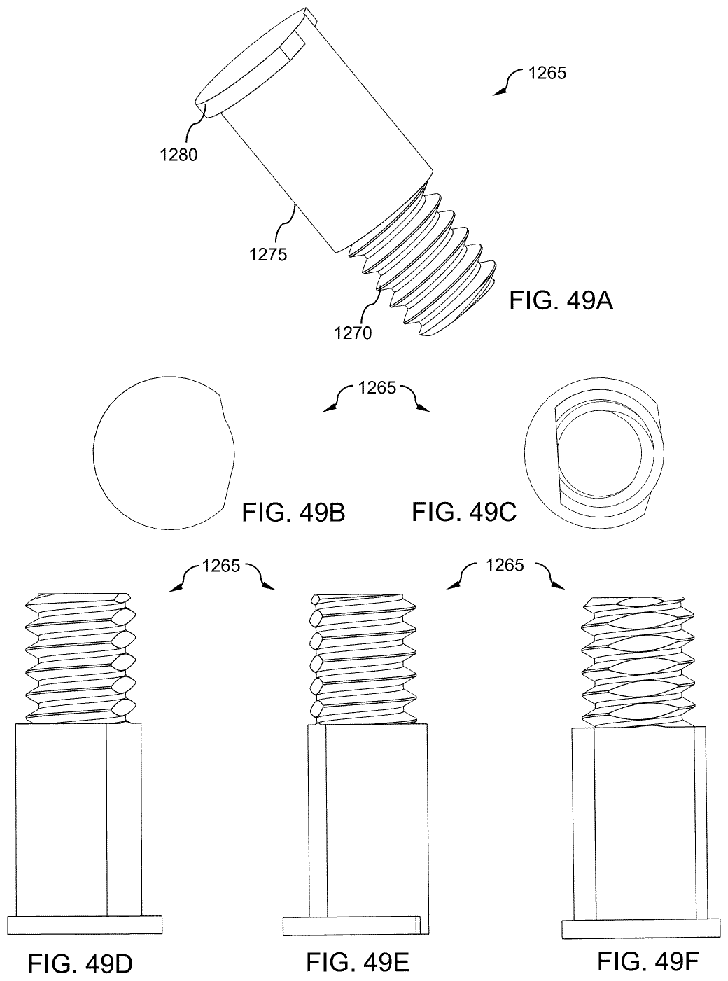

FIGS. 49A-49F illustrate several plan views of a PASA or DMAA pin of the fourth actuation mechanism 25, 90, 120, 205. The PASA or DMAA pin comprises a flange, and a shank body. The shank body comprises a first portion and a second portion. The first portion comprises a first portion shape, the first portion shape is a “D” shape or semi-circle. The second portion comprises a threaded portion. The PASA or DMAA pin is inserted into the first and second through-holes of the translation screw housing until the flange hits or contacts a bottom surface of a portion of the recess on the displacement screw housing base. The threaded portion is sized and configured to be disposed with the wingnut. The threaded portion engages with the wingnut.

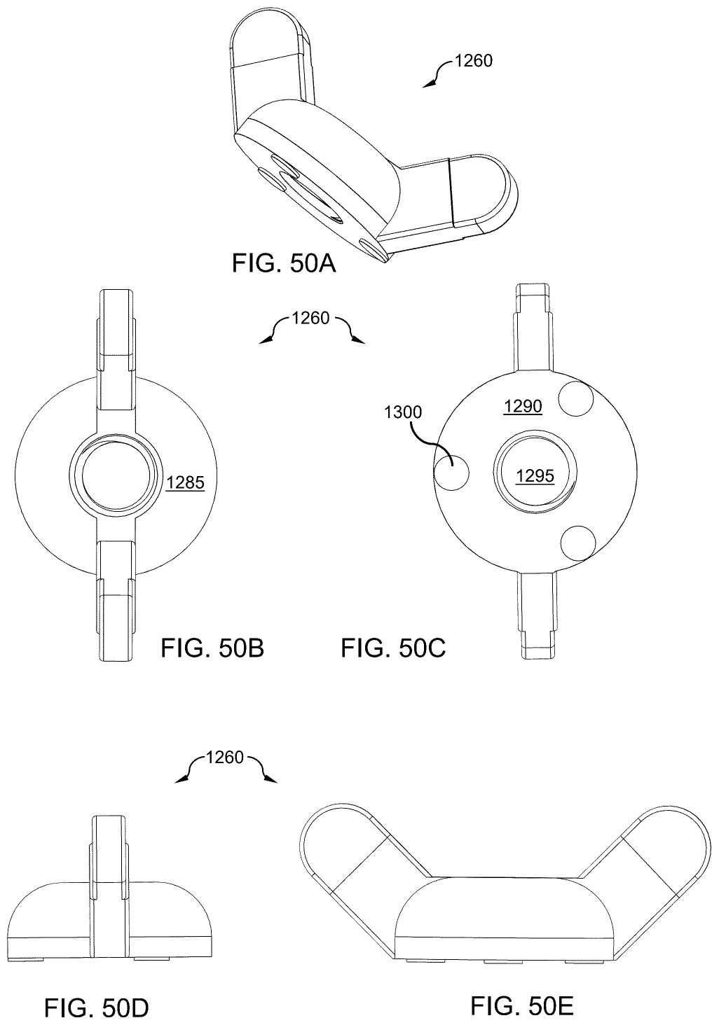

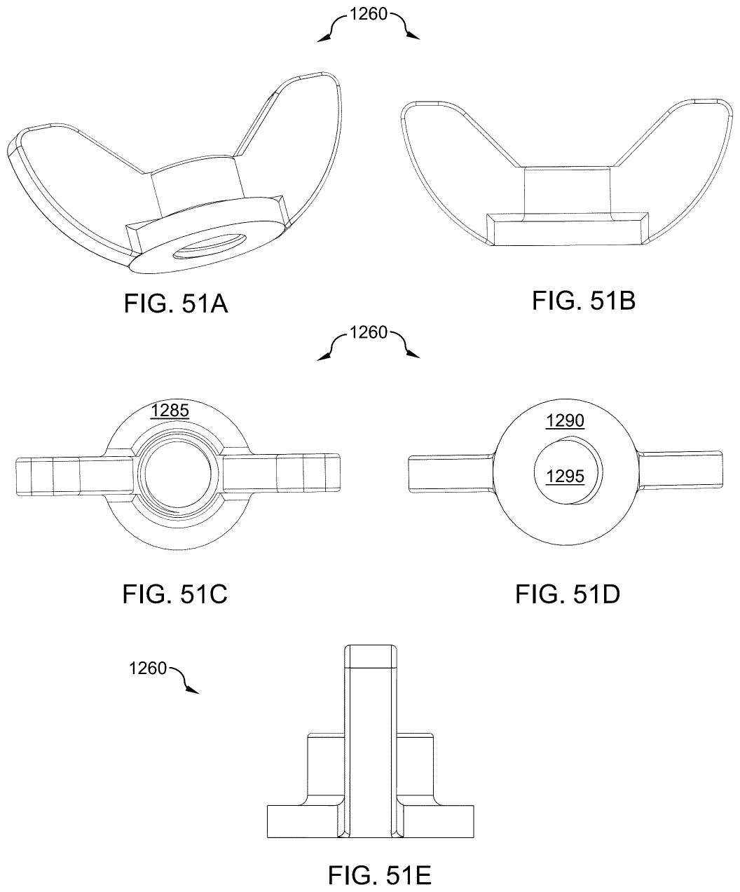

FIGS. 50A-50E and 51A-51E illustrate several plan views of different embodiments of a DMAA knob or wingnut 1260 of the fourth actuation mechanism 25, 90, 120, 205. The wingnut may comprise a head, a first wing, a second wing and a threaded opening. The first and second wing are disposed onto the head 180 degrees apart and/or diametrically opposed. The first and second wing allow the physician to rotate the wingnut easily without tools. The threaded opening is sized and configured to receive the threaded portion of the PASA or DMAA pin. The head of the wingnut further comprises a plurality of pads. The plurality of pads is disposed onto a bottom surface of the head of the wingnut. The plurality of pads is disposed circumferentially onto a bottom surface of the head of the wingnut.

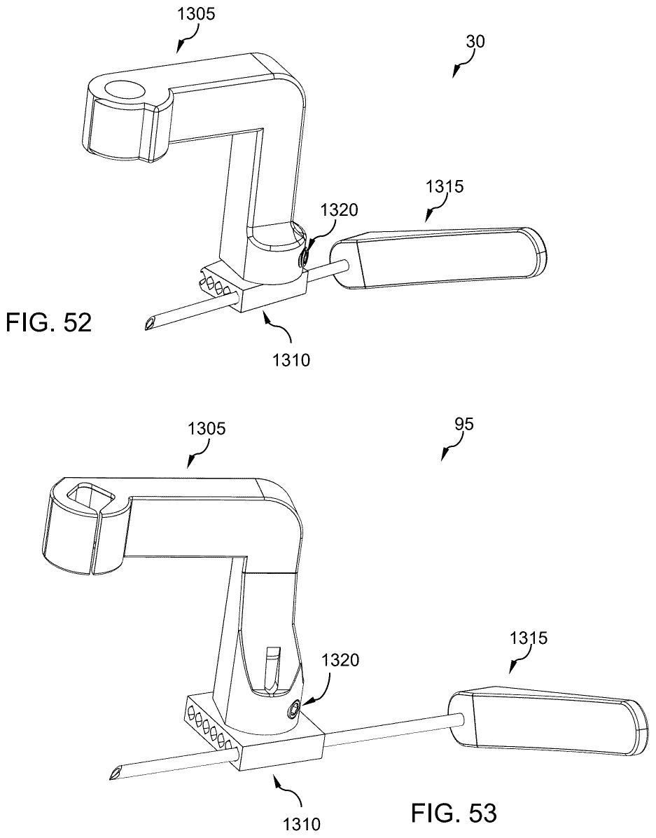

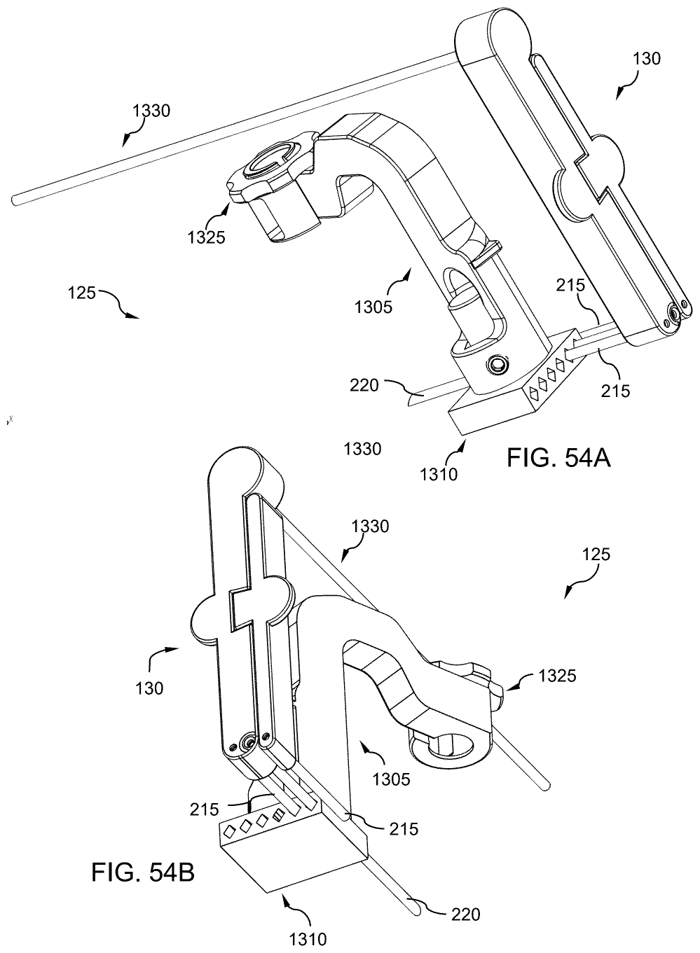

FIGS. 52-53 and 54A-54B illustrate isometric views of different embodiments of the fixation screw guide arm assembly 30, 95, 125. The fixation guide arm assembly may allow the physician to achieve surgical stabilization of the capital fragment with internal medially placed one or more fixation screws by targeting the trajectory of the fixation. The fixation guide assembly comprises a guide arm, a drill sleeve, a sleeve base, and a set screw. The fixation guide assembly may further comprise an alignment columns assembly. The fixation guide may further comprise one or more guide sleeves.

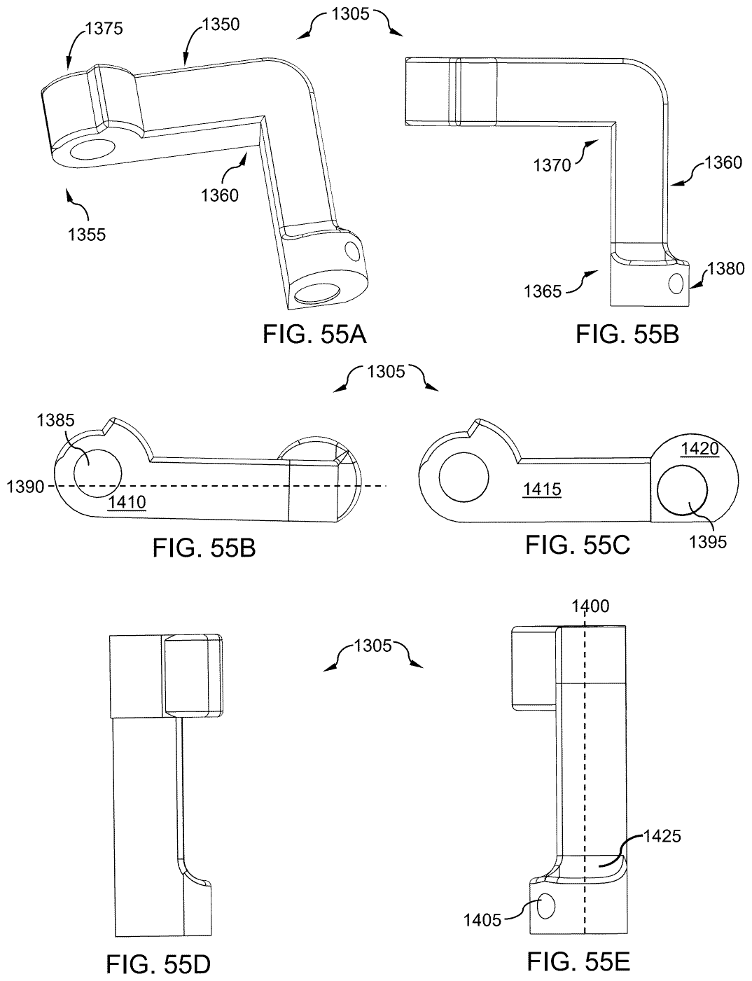

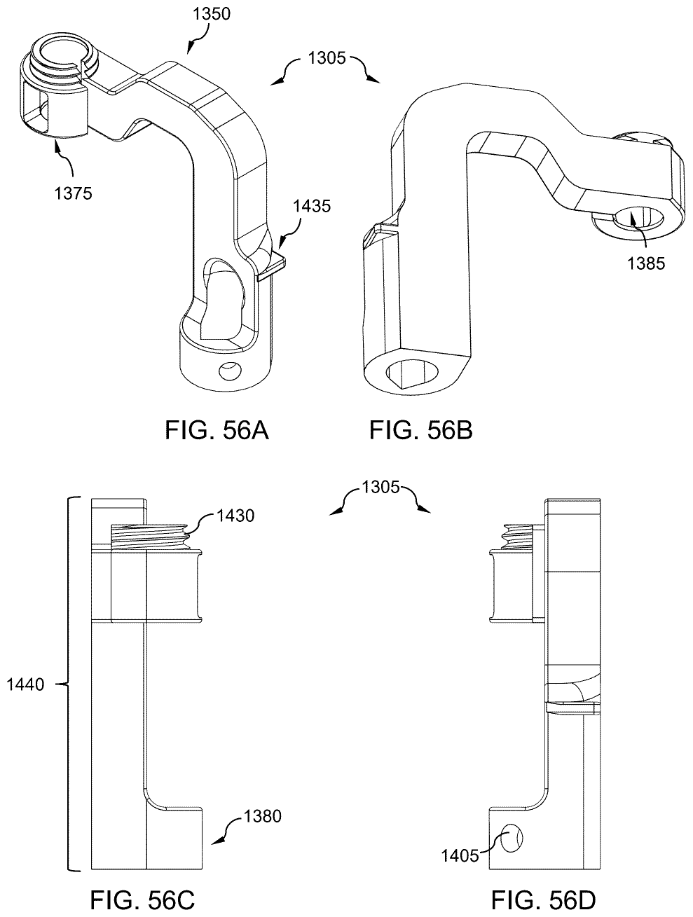

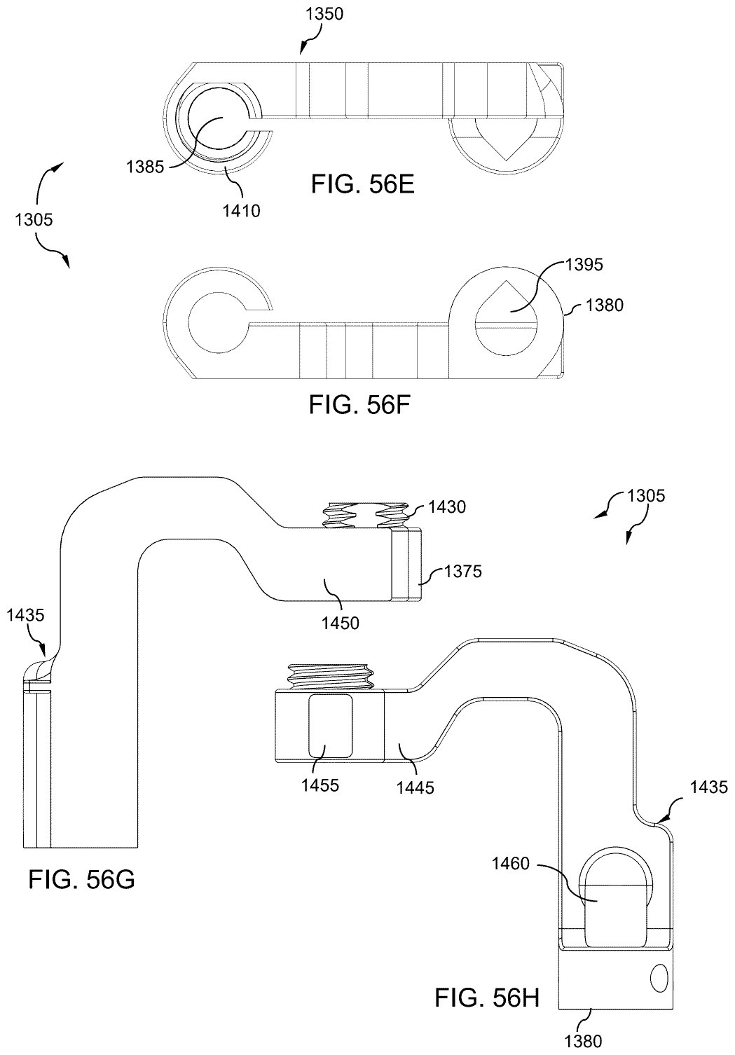

FIGS. 55A-55E and 56A-56H illustrate various plan views of different embodiments of a guide arm 1305. The guide arm comprises a support member, a first base and a second base. The support member comprises an “L” shape. The support member includes a first portion or member and a second portion or member. The first portion includes a first portion longitudinal axis, and the second portion includes a second portion longitudinal axis. The first portion or member is coupled to the second portion or member. The first portion or first member is disposed or coupled to the second portion or member perpendicular to the first portion longitudinal axis. Alternatively, the first portion or member extends perpendicular to or substantially perpendicular to the second portion or member.

The first base is coupled to the first portion or member of the support member. The first base comprises a top surface, a bottom surface and a first opening. The first base may further comprise a threaded post. The first opening extends through the top surface to the bottom surface of the first base. The first opening is sized and configured to receive a portion of the stem of the tarsal base of the first actuation mechanism. This allows the guide arm to move or rotate around the longitudinal axis of the stem of the tarsal base at a range of 0 degrees to 180 degrees; 0 degrees to 150 degrees; 0 degrees to 100 degrees; 0 degrees to 75 degrees; 0 degrees to 60 degrees; and/or 0 degrees to 45 degrees. Alternatively, the guide arm can move relative to the tarsal base of the first actuation mechanism. The first base of the first portion or member may comprise a cylindrical shape and/or a “C” shape. The first base may further comprise a window. The window extends from a first side surface towards a second side surface. The window is used to view the stem or post of the tarsal base as it slides along the vertical axis of the first opening.

In one embodiment, the first base further comprises a threaded post. The threaded post extends from a top surface of the first base. The threaded post extends upwardly from the top surface of the first base. The first opening extends through the threaded post. The threaded post comprises a tapered shape and/or is tapered. The first opening of the first base is sized and configured to receive the receive the threaded knob. The stem or post of the tarsal base is slidable along the vertical or axial axis of the first opening of the first base. When the threaded knob is screwed onto the threaded post of the first base of the first portion or first member of the guide arm, a portion of the first base will move towards the stem or post of the tarsal base and engage or grip the post of the tarsal base tightly, thus preventing or reducing movement. Alternatively, when the threaded knob is screwed onto the threaded post of the first base, the first base moves inwardly from a first position, the first position includes a first opening with a first diameter to a second position, the second position allows first opening of the first base to change to a second diameter, the second diameter being smaller than the first diameter, and allow gripping or clamping of the stem or post of the tarsal base. In another embodiment, when the threaded knob is screwed onto the threaded post of the first base, the threaded knob forces the first base to an open position and a closed position.

The second base is coupled to the second portion or member of the support member. The second base comprises a top surface, a bottom surface, a outer surface, second opening and a bore. The bore of the second base extends from the bottom surface of the second base towards the top surface of the second base. The bore includes a bottom surface and an inner diameter. The inner diameter of the bore is sized and configured to receive dowel or pin of the sleeve jig. The second opening is disposed transversely to the longitudinal axis of the second portion longitudinal axis onto the outer surface of the second base. The second opening is in communication with the bore. The second opening is threaded. The second opening is sized and configured to receive the set screw to fixate or lock the sleeve base or jig relative to the guide arm and prevent rotational movement. The second portion or member of the support member may further comprise a tab. The tab may extend outwardly from a surface of the second portion of the support member. The tab may extend transversely from a surface of the second portion of the support member. The tab may be disposed between a first end and a second end of the second portion of the support member. The tab is sized and configured to receive a portion of a physician's or surgeon's hand to help remove the guide arm from the stem or post of the tarsal base.

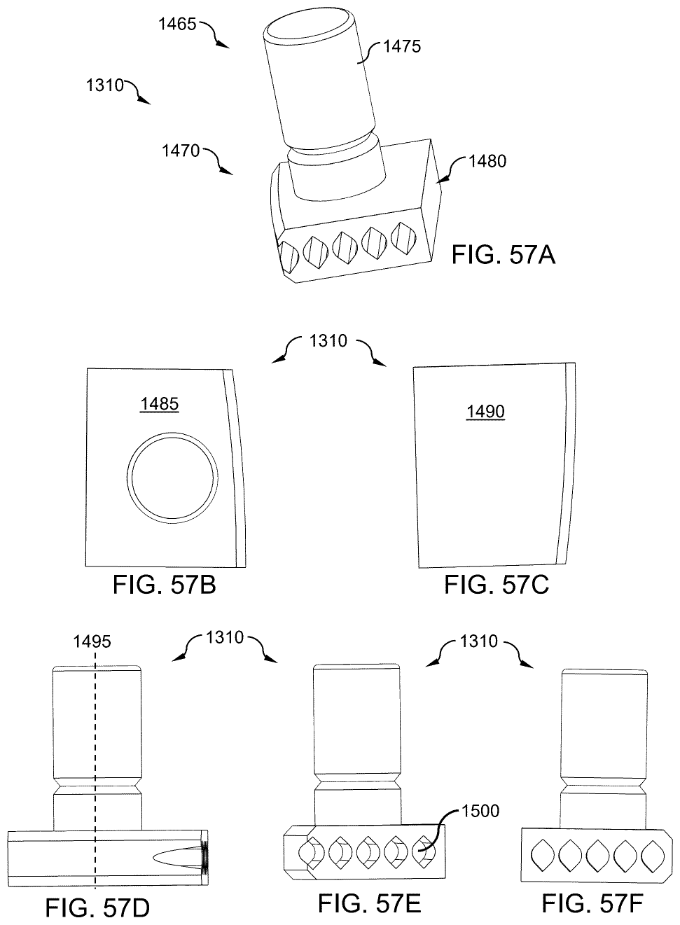

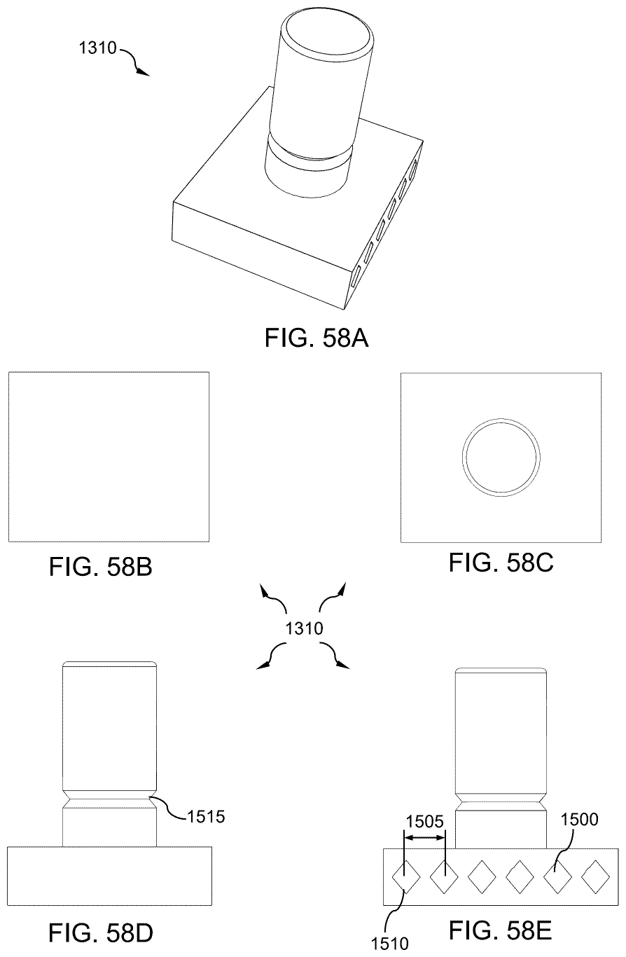

FIGS. 57A-57F and 58A-58E illustrate various plan views of different embodiments of a guide sleeve jig or guide sleeve base 1310 for the fixation guide arm assembly 30, 95, 125. The sleeve jig comprises a sleeve base or platform and a sleeve pin. The sleeve base comprises a top surface, a bottom surface, a first side, a second side, a shape, and a plurality of openings. The shape may comprise a circle, oval or polygonal shape. The polygonal shape may include a triangle, square, rectangle, pentagon, and/or hexagon. In one exemplary embodiment the shape is rectangular or substantially rectangular. The plurality of openings are disposed perpendicular to the longitudinal axis of the sleeve jig. The plurality of openings extends from a first side through the second side of the sleeve base. The plurality of openings are sized and configured to receive the drill sleeve and/or guide sleeves. Each of the plurality of openings are spaced apart from each other. The spacings may comprise at least 2 mm and/or the spacings may comprise at least 4 mm.

The sleeve dowel or pin is disposed onto a top surface of the sleeve base and extends upwardly. The sleeve dowel or pin comprises an outer surface or outer diameter. The dowel pin is solid or hollow. The dowel or pin outer diameter or outer diameter surface is sized and configured to be disposed into the first opening of the first base of the guide arm. The sleeve dowel or pin further comprises a trough or channel. The trough or channel surrounds the outer surface or outer diameter of the dowel or pin. The trough or channel is sized and configured to receive a portion of the set screw. The insertion of the sleeve dowel or pin into the bore of the second base of the guide arm allows the physician to rotate the sleeve jig until a desired trajectory is found, then the sleeve jig may be fixed or locked to the guide arm with the set screw.

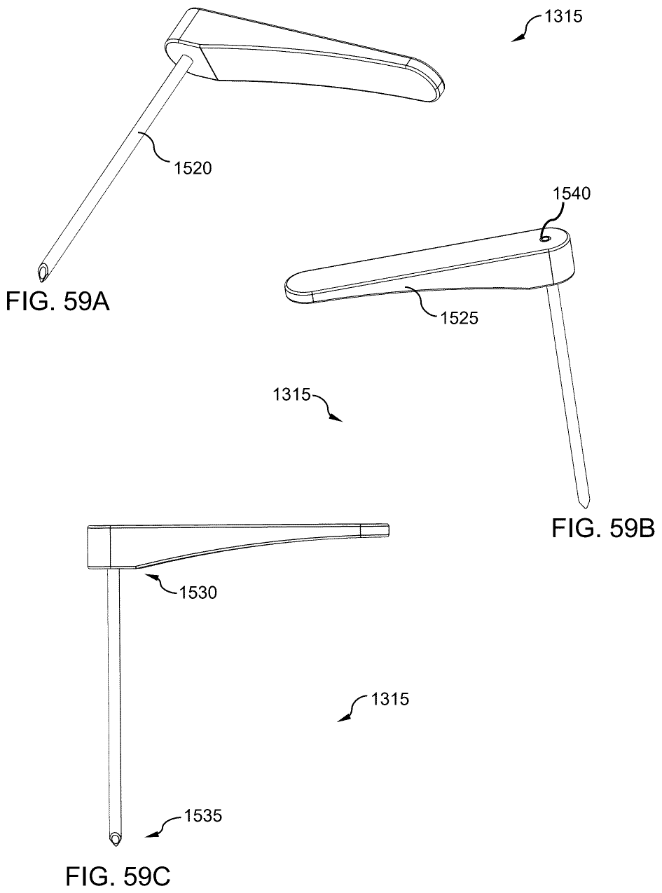

FIGS. 59A-59C illustrate various plan views of one embodiment of a drill sleeve of the fixation guide arm assembly 30, 95. The drill sleeve comprises a cannula and a handle. The cannula comprises a first end, a second end, and a body. The body includes a hollow, cylindrical tube. The handle is coupled to the handle at the first end of the cannula. The second end of the cannula may comprise beveled or sharp tip to allow easier introduction into the tissues and bone of the foot. The body of the cannula further comprises a lumen. The lumen extends from the first end through the second end. The lumen is sized and configured to receive one or more fixation screws. The body of the cannula may comprise a material, the material may include a polymer or plastic.

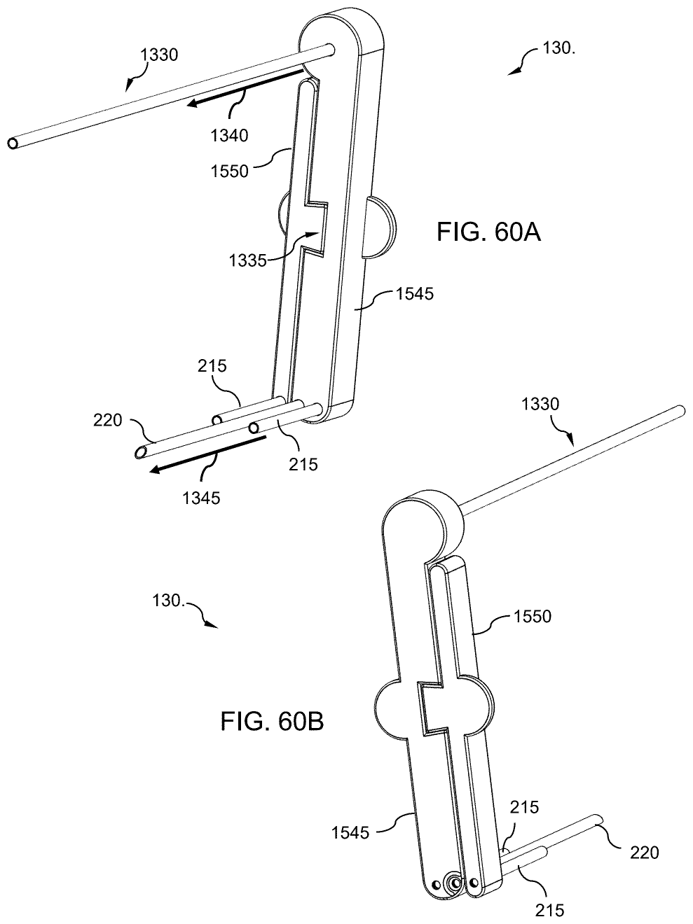

FIGS. 60A-60B depict isometric views of one embodiment of an alignment column assembly 130 of the fixation guide assembly 125. The alignment column assembly comprises a first column, a second column, an alignment rod, and a drill sleeve. The alignment column assembly may further comprise one or more guide sleeves. The first column is coupled or removably coupled to the second column. The first column slidably engages with the second column. The coupling may comprise a dove-and-tail coupling or joint. The alignment rod is removably connected to the first column. The alignment rod extends from a first end of the first column. The alignment rod extends perpendicularly or transversely from a first end of the first column. The alignment rod comprises an alignment rod length. The alignment rod and/or the alignment rod length extends from a medial side of the foot and across a portion of the body of the first metatarsal to view the desired trajectory of the fixation screws into the capital fragment. One or more guide sleeves and/or a drill sleeve extend from a second end of the first and second columns. One or more guide sleeves and/or a drill sleeve extend perpendicular and/or transversely from a second end of the first and second columns.

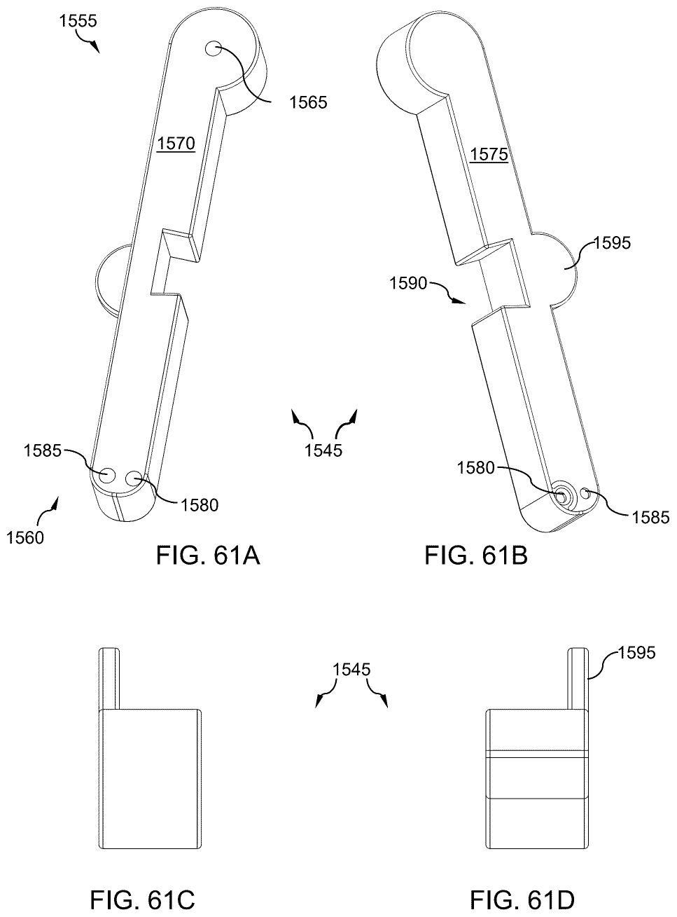

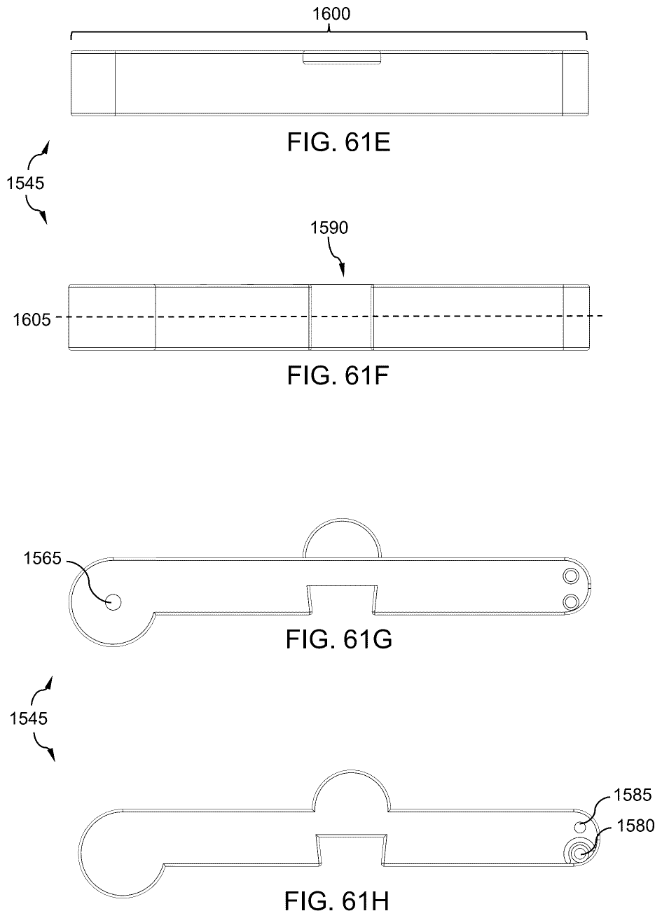

FIGS. 61A-61H depict various views of one embodiment of a first column 1545. The first column comprises first end, a second end, a pin socket and a tab. The first column further comprises a first opening, a second opening and a third opening. The pin socket is disposed between the first end and a second end. The pin socket is sized and configured to receive a pin element or engages with the pin element of the second column to create a dove-tail joint. The tab is disposed between the first end and the second end. The first opening is disposed at or adjacent to the first end. The first opening extends from a first or front surface toward the second or back surface. The first opening is sized and configured to receive a portion of the alignment rod. The second and third opening are disposed at or adjacent to the to the second end. The second and third opening are spaced apart. The spacings may comprise at least 2 mm or greater and/or at least 4 mm or greater. The first column may further comprise an indicator. The indicator may be disposed adjacent to the second and/or third opening. The second and/or third openings are sized and configured to receive a portion of one or more drill sleeves and/or guide sleeves. The second and/or third opening extend from the first or front surface through the second or back surface.

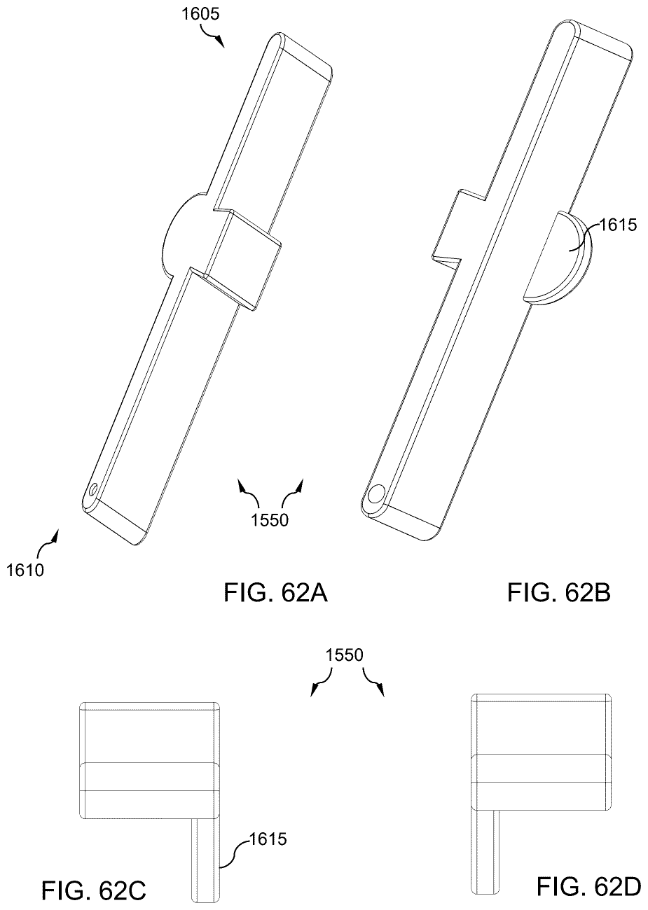

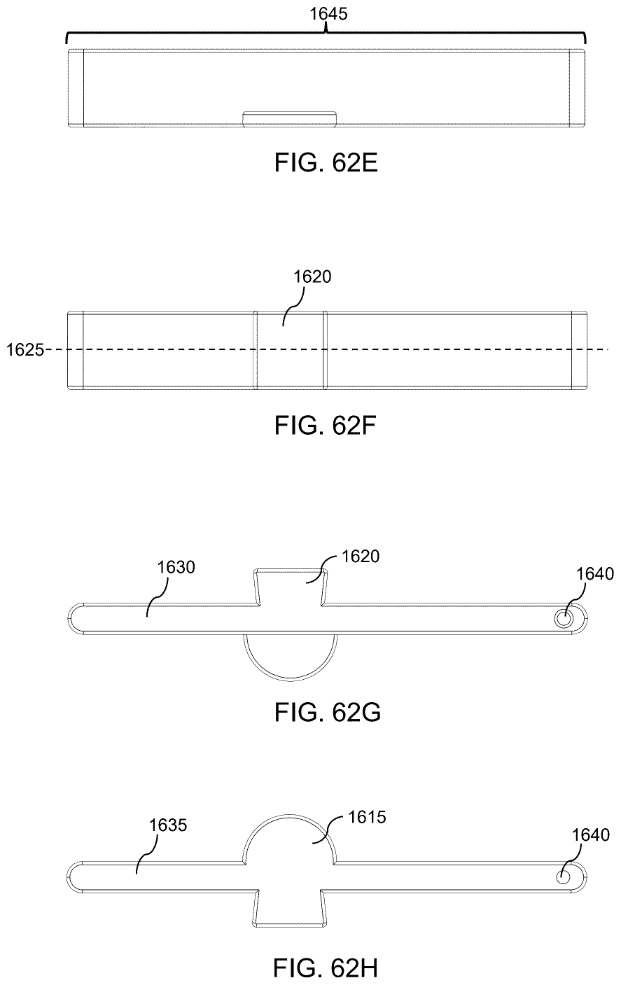

FIGS. 62A-62H depict various views of one embodiment of a second column 1550. The second column comprises a first end, a second end, a tab and a pin element. The second column further comprises an opening. The pin element is disposed between the first end and a second end. The pin element is sized and configured to be disposed within the pin socket of the first column to create a dove-tail joint. The tab is disposed between the first end and the second end. The opening is disposed at or adjacent to the second end. The opening extends from a first or front surface through the second or back surface. The opening is sized and configured to receive one or more drill sleeves or guide sleeves. The second column may slidably engage with the first column to create a dove-tail joint. The second column may be detached and removed from the first column to provide ease of access for the physician. Alternatively, the second column may be detached and rotated inferiorly to provide ease of access for the physician.

The bunion guide system, the first actuation mechanism, the second actuation mechanism, the third actuation mechanism, and the fourth actuation mechanism may comprise a material. The material may include metal or polymer materials. The polymer materials may comprise a translucent, radiolucent or radiopaque material. In one exemplary embodiment, the polymer material comprises a radiolucent polymer material. The radiolucent polymer materials are not dense enough to keep the x-rays from shining through them, so the materials appear “clear” on an x-ray or imaging, and prevent obscuring of crucial patient anatomy. The radiolucent polymer material may comprise carbon fiber or carbon fiber reinforced polymers.

One or more of the actuator mechanisms may comprise an indicator, the indicator may include a color, a symbol and/or a directionality. In one exemplary embodiment, the first actuator mechanism comprises a first indicator, the second actuator mechanism comprises a second indicator, the third actuator mechanism comprises a third indicator, and the fourth actuator mechanism comprises a fourth indicator. Each of the first, second, third and fourth indicators comprise a different colored indicator to differentiate the actuator mechanisms. Alternatively, each of the first, second, third and fourth indicators comprise a directionality. Each of the first, second, third and fourth indicators comprises a different color and directionality indicator.

### Methods of Correction or Use

The bunion correction guide system or the bunion guide system is desirably positioned on a portion of the superior surface of a patient's foot. The bunion guide system's positioning on the foot surface provides many advantages, including, but not limited to aiding in stability during the use or control of the various actuation mechanisms; reduction of forces during use or control of the actuation mechanisms and facilitates more accurate alignment of head of the first metatarsal; and/or reducing surgical workspace profile.

Accordingly, the bunion correction guide comprises a plurality of fixed or modular actuation mechanisms that may be controlled, adapted or directed at treating mild to moderate hallux valgus angle deformities by allowing successful correction of the deformity by: normalizing the tension of the tendons and muscles; establishing a congruous MTP joint, maintaining or increasing the first MTP joint range of motion; reducing the intermetatarsal angle (IMA); realigning the sesamoids underneath the metatarsal head to restore the ability to bear weight and distribute load properly; and/or realigning the hallux to a rectus position. Nevertheless, the ultimate goal is the elimination of pain, restoration of a congruous metatarsophalangeal joint, realignment of the hallux into its proper anatomical position (including the rotation of sesamoids), and preservation of joint motion.

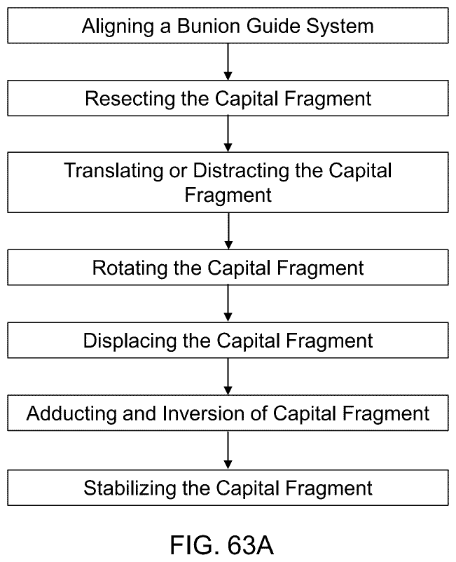

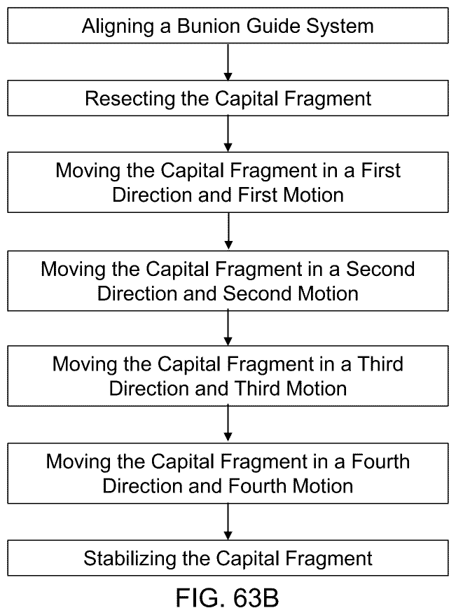

FIG. 63A depicts one embodiment of a method of correcting a bunion deformity. The method of correcting a bunion deformity comprises the steps of: aligning a bunion guide system; resecting the capital fragment; translating or distracting the capital fragment; rotating the capital fragment; displacing the capital fragment; adducting and inversion of the capital fragment; and stabilizing the capital fragment. The method may further comprise: a preoperative assessment of the bunion deformity. The method may further comprise the step of correcting the medial prominence of the body of the first metatarsal after final hallux valgus correction.

FIG. 63B depicts an alternative embodiment of a method of correcting a bunion deformity. The method of correcting a bunion deformity comprises the steps of: aligning a bunion guide system; resecting the capital fragment; moving the capital fragment in a first direction and a first motion; moving the capital fragment in a second direction and a second motion; moving the capital fragment in a third direction and a third motion; moving the capital fragment in a fourth direction and a fourth motion; and stabilizing the capital fragment. The method may further comprise: a preoperative assessment of the bunion deformity. The method may further comprise the step of correcting the medial prominence of the body of the first metatarsal after final hallux valgus correction.

In one embodiment, the method of correcting a bunion deformity further comprises obtaining a preoperative assessment (not shown). The preoperative assessment may comprise evaluation or assessment of a patient's history, laboratory studies, weight bearing and non-weight bearing radiographic evaluation, palpations of the bunion to localize the exact area of tenderness and/or gait analysis.

Proper radiographic evaluation of the hallux abductus valgus deformity requires standard preoperative weight-bearing views using one or more imaging techniques. Standard preoperative views should consist of weight-bearing anteroposterior (AP), lateral, sesamoid axial, and lateral oblique projections should be obtained in the angle and base of gait. The lateral views together will allow the practitioner to accurately measure traditional relationships and identify positional and structural components of the deformity. The oblique projections demonstrate hypermobility from lack of parallelity between the first and second metatarsals. The oblique projection will also act as a standard for assessment of pre- and postoperative correction of the deformity. The sesamoid axial projection will aid in evaluating degenerative changes noted within the sesamoid apparatus. The plantar crista of the first metatarsal head may also be viewed and evaluated for erosive changes that may accelerate the deformity. The collection of these radiographic images may be used to judge or grade the severity of the bunion deformity using different angular deformity measurements.

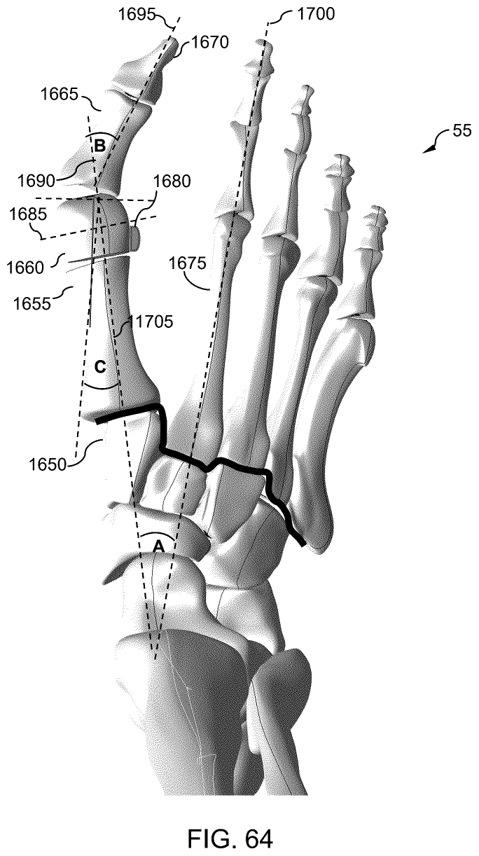

FIG. 64 depicts a top view of one embodiment of a hallux valgus deformity. The foot bones can be grouped into three sets: the tarsal bones, the metatarsals and the phalanges. The first metatarsal bone, the shortest, thickest and strongest metatarsal, links to the big toe. The big toe is made up of two phalanges, the proximal phalanx and the distal phalanx. Each metatarsal has a head (closest to the toes), body and base (closest to the ankle). They articulate proximally to the tarsal bones. The first metatarsal bone links to the medial and intermediate cuneiform, the second metatarsal to all three cuneiforms, the third metatarsal to the lateral cuneiform, the fourth metatarsal to the lateral cuneiform and cuboid foot bones and the fifth metatarsal bone to the cuboid.

The final bony anatomic considerations of the foot bones involve the sesamoids. They have an important function for weightbearing and reducing load-per-unit area on the first metatarsal head. The sesamoids are located in the flexor hallucis brevis (FHB) tendon and lie under the first metatarsal (MT) head or capital fragment. The plantar aspect of the first metatarsal head has a longitudinal intersesamoid ridge in its center, termed the crista. The sesamoids lie on either side of this ridge as they articulate with the plantar surface of the first metatarsal head. Normally, they should be centered under the first MT head on the standing anteroposterior (AP) radiograph of the foot.

As the great toe develops a valgus deformity, the first metatarsal may show dorsiflexion, adduction, and inversion resulting in the first metatarsal head to deviate medially, and rotation occurs at the metatarsophalangeal (MTP) joint. As the deformity progresses over time, the tibial sesamoidal ligament becomes functionally elongated as it adapts to stress placed on the medial side of the first metatarsophalangeal joint. The fibular sesamoidal ligament, conversely, functionally shortens along with the other lateral soft tissue structures. The first metatarsal rotates slightly at the metatarsal cuneiform articulation. In a pronated foot, this slight rotation of the metatarsal allows for an inversion or varus rotation of the first metatarsal head relative to the sesamoids. The hallux now moves in the opposite direction of the first metatarsal head, which accounts for the valgus or rotational component of the deformity. As the amount of hallux eversion increases over time, the tibial, intersesamoidal, and fibular sesamoidal ligaments continue to adapt functionally to the deformity.

The AP projection and other projections are used to determine the various angular deformity measurements. The angular deformity measurements include: intermetatarsal angle (IMA)(“A”), hallux abductus angle (HAA) or hallux valgus angle (HVA) (“B”), and proximal articular set angle (PASA)(“C”). The angular deformity measurements may further include metatarsus adductus angle (MAA), and hallux abductus interphalangeus angle (HAIA), metatarsophalangeal angle (MTPA), distal articular set angle (DASA), distal metatarsal articular angle (DMAA), as well as the first metatarsal length, sesamoid position, condition of the first metatarsophalangeal (MTP) joint, bone stock, first metatarsal base, hallux rotation, and medial metatarsal head enlargement (see the images below), and/or any combination thereof.

The IMA angle is a measurement used to assess hallux valgus. The IM angle is formed by the angle produced at the intersection of the longitudinal bisection of the shafts or body of the first and second metatarsal. Abnormally increased IM angle approaches values equal to or greater than 9 degrees. A mild abnormality is a range between 9 to 11 degrees; a moderate abnormality is range between 11 to 16 degrees; and/or a severe abnormality is a range of greater than 16 degrees.

The HVA angle is a measurement used to assess the presence and severity of the alignment of the first metatarsophalangeal joint. The hallux valgus angle, B, is formed by the intersection of a line drawn through the long axis of the first metatarsal and the long axis of the proximal phalanx. A normal hallux valgus angle is one that measures less than 15 degrees. Mild deformity is present when this angle measures between 15 degrees and 20 degrees. A moderate deformity is present when this angle measurement is between 20 to 40 degrees. The deformity is categorized as severe when the angle measures greater than 40 degrees.

The proximal articular set angle (PASA), C, is another valuable measurement of structural deformity within the metatarsal head. This angle is formed by the intersection of the perpendicular line to the line passing through the two medial and lateral end points of the articular surface of the head of the first metatarsal and the longitudinal axis of the first metatarsal. An abnormal increase in PASA may demonstrate the location of a structural deformity in the head of the metatarsal, and it may progressively increase secondarily to structural adaptation of the articular cartilage surface as the deformity progresses. A normal PASA is one that measures less than 8 degrees in the rectus foot, therefore, an abnormal PASA comprises equal to or greater than 8 degrees.

The distal metatarsal articular angle (DMAA) is measured between the distal articular surface and the perpendicular line to the longitudinal axis of the first metatarsal. The DMAA identifies MPT joint incongruity. It is considered non-pathological or normal at up to 10⁰ in angle. The correction of DMAA could alleviate the surgeon from performing an Akin Osteotomy or other appropriate osteotomies to further align the Great Toe.

The collected data is analyzed to assign the severity of the hallux valgus deformity to assign a stage or grade. The severity may be categorized into at least four stages, stages 1 through 4. Stage 1 is defined lateral displacement of the hallux at the MTP joint. Stage 2 is defined as hallux abduction progresses (hallux pressing against the second toe). Stage 3 is defined as further subluxation occurs at the first MTP joint, with increased IMA. Stage 4 is defined as the first MTP joint is partially or completely dislocated. Alternatively, the severity may be classified into normal, mild, moderate or severe based on the IMA and/or HVA. Normal may be considered as HVA less than 15 degrees and IMA less than 9 degrees. Mild may be considered as HVA between 15 to 30 degrees and IMA between 9 to 13 degrees. Moderate may be considered as HVA between 30 to 40 degrees and IMA between 13 to 20 degrees. Severe may be considered HVA over 40 degrees and IMA over 20 degrees.

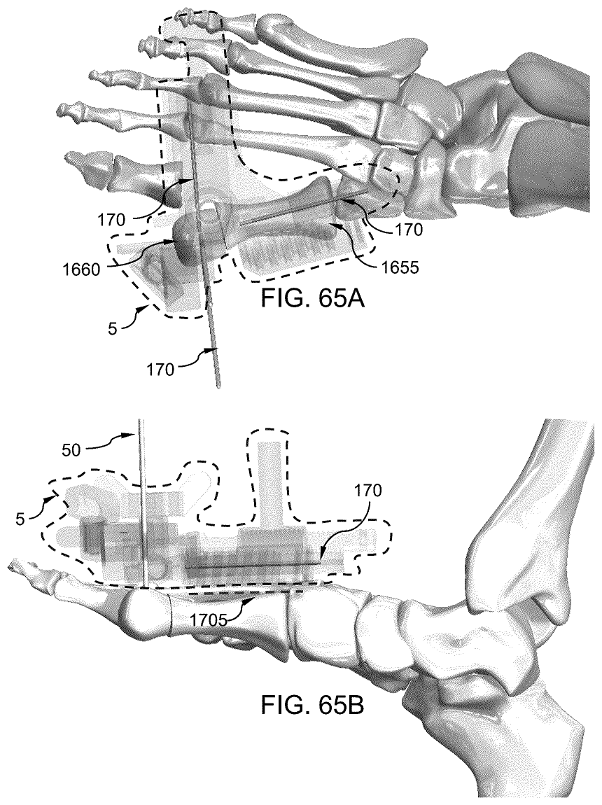

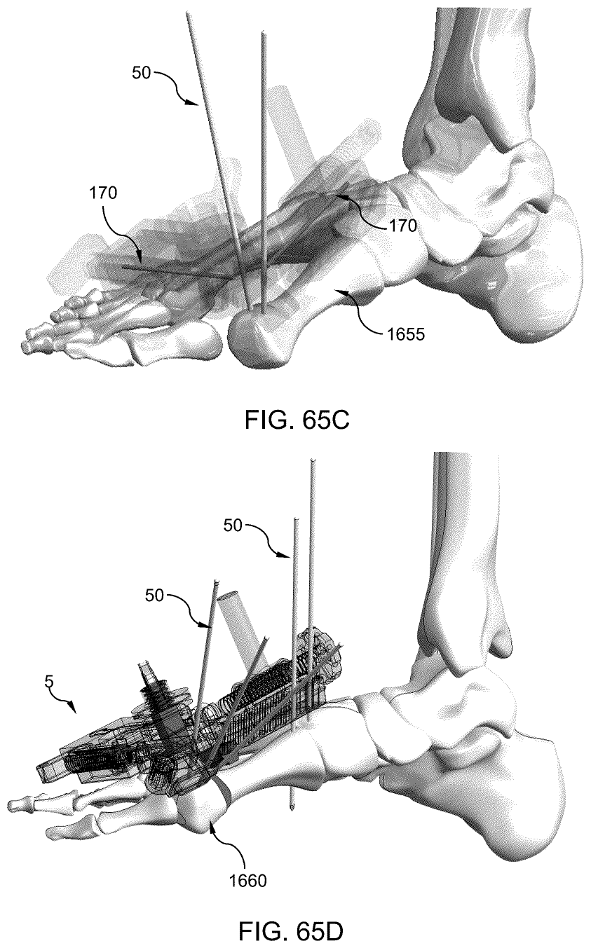

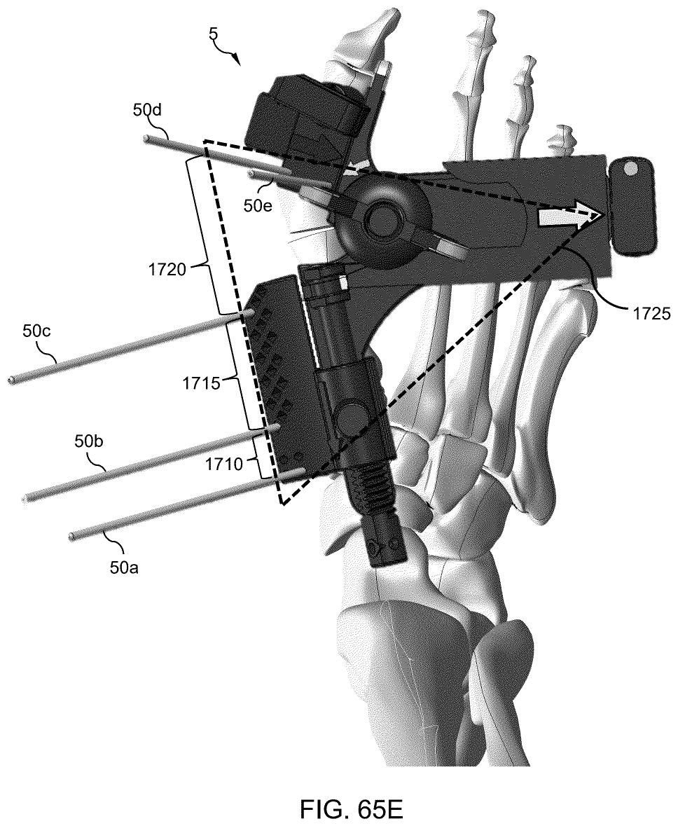

In one embodiment, the method of correcting a bunion deformity comprises aligning the bunion guide system as shown in FIGS. 65A-65E. The step of aligning the bunion guide system comprises the steps of: obtaining initial trajectory axis' of a patient's foot; positioning the bunion guide system; and/or anchoring or securing the bunion guide system.

The step of obtaining an initial trajectory axis' of a patient's foot requires marking a first and second trajectory axis on a patient's foot. The patient is brought to the OR and placed in the supine position on a regular radiolucent table. Under X-ray or imaging guidance, the physician should identify and mark the first trajectory axis and the second trajectory axis. The longitudinal axis of the first metatarsal is identified and marked out with a surgical marker, the longitudinal axis of the first metatarsal body is the first trajectory axis. The transverse axis or the second trajectory axis of the capital fragment or the head of the first metatarsal is identified and marked out with a surgical marker. Alternatively, the physician may mark a third trajectory axis with a surgical marker.

The step of positioning the bunion guide system allows the physician to position the bunion guide system relative to the first and second trajectory axis. Alternatively, the step of positioning the bunion guide system allows the physician to position the bunion guide system relative to the first, second and third trajectory axis. The physician should dispose or position the assembled bunion guide system onto the prepped skin surface of a patient's foot. The patient's foot may have different surface morphologies that may affect the balance and stability of the bunion guide system during operation of its actuators. It is desired that the physician ensures proper balancing of the bunion guide system onto the patient's foot to facilitate reduced or easier forces while in operation. In one embodiment, a stabilizing block may be introduced and/or removably coupled to a portion of the bunion guide system. More specifically, a stabilizing block may be removably coupled to a portion of the tarsal base and/or the anchor extension body of the tarsal base. At least a bottom portion of the stabilizing block will contact the uneven surface of the patient's foot providing a more balanced and/or planar working surface for the guide actuators. It can prevent toppling over, tipping sideways, and migration (e.g. making it stationary).

Accordingly, the physician should align or substantially align the first radiopaque marker disposed within the tarsal base or the radiopaque indicator within the tarsal base with the first trajectory axis on the body of the first metatarsal. The physician should align or substantially align the second radiopaque marker or radiopaque indicator disposed within a portion of third actuation system (e.g., within the displacement or translation screw housing) with the second trajectory axis. If a third trajectory axis is available, the physician should align or substantially the third radiopaque marker or radiopaque indicator disposed within the tarsal base transverse to the first radiopaque marker to the third trajectory axis. If desired, the guide slot of the assembled bunion guide system should or may be positioned over the desired osteotomy location to provide the physician a “viewing window or viewing boundary.”

Alternatively, the physician should position the marked first trajectory axis parallel or substantially parallel to the first trajectory axis. The first trajectory axis marker may match or substantially match or align with the longitudinal axis of the first metatarsal longitudinal axis in the dorsal and lateral views. The second trajectory axis should match, align or be parallel to and/or substantially match, align or be parallel marked second trajectory axis. Alternatively, the second trajectory axis should match the transverse axis of the two converging markers in the dorsal and lateral view.

The step of aligning the bunion guide system may further comprise the steps of securing, stabilizing or fixating the bunion guide system relative to the foot. Once or more anchoring pins may extend through a portion of the bunion guide and the foot to provide proper stabilization of the bunion guide system. In one embodiment, one or more anchoring pins may be desirably positioned along one or more locations on the foot. The one or more locations on the foot may include the metatarsals, the phalanges, the tarsals and/or any combination thereof. The tarsals may include the cuneiform bones, the navicular, the cuboid and/or any combination thereof. The metatarsals may include the first metatarsal, the second metatarsal, the third metatarsal, the fourth metatarsal, and/or the fifth metatarsal. The first metatarsal may include the capital fragment and the body of the first metatarsal. The cuneiform bones may include medial cuneiform, intermediate cuneiform and/or lateral cuneiform.

In another embodiment, a first one or more anchor pins may be positioned onto a portion of the medial cuneiform bone and through a proximal end of the anchor extension body of the tarsal base. Alternatively, the first one or more anchoring pins may be positioned on a portion of the medial cuneiform that is near or adjacent to the tarsometatarsal joint and through a first location on the anchor extension body of the tarsal base. A second one or more anchor pins may be positioned on the proximal portion of the first metatarsal and extend through a second location on the anchor extension body of the tarsal base to form a first distance, d1. A third one or more anchor pins may be positioned distal portion of the first metatarsal and proximal to the capital fragment and extend through a third location on the anchor extension body of the tarsal base to form a second distance, d2. A fourth one or more anchor pins may be positioned onto a portion of the capital fragment head and extends through the rotation block of the second actuator to form a third distance, d3. Each of the distances, d1, d2, d3, may comprise a distance of 8 mm to 35 mm.

In another embodiment, a first anchoring pin may be extended through the orthogonal registration opening on the tarsal base of the first actuation mechanism. This allows the physician to make the initial registration for alignments. The first anchoring pin may extend through the distal portion of the body of the first metatarsal. The first anchoring pin may extend perpendicular from the longitudinal axis of first metatarsal and/or the first trajectory axis marked by the physician. This allows the physician to desirably rotate the bunion guide system around the first anchoring pin to for positioning. This first anchoring pin may alternatively be placed during the step of positioning the bunion guide system as discussed above.

A second anchoring pin may extend through the capital fragment openings located on a portion of the 2^{nd} actuation mechanism to be secured on a region of the capital fragment of the first metatarsal. The region of the capital fragment may include a superior region and/or a dorsal region. The dorsal region may desirably help avoid puncturing or inserting through the tendon (e.g., extensor hallucis longus tendon) that runs parallel to the longitudinal axis of the first metatarsal and is centrally located on the superior surface. Otherwise, the physician may need to carefully straddle the tendon to avoid complications. More specifically, the second anchoring pin may extend through the capital fragment openings disposed onto the rotation block of the second actuation mechanism. If desired a third anchoring pin and/or fourth anchoring pin may be extended through the capital fragment openings located on the rotation block of the second actuation mechanism.

Alternatively, a second anchoring pin may extend through a first capital fragment opening onto a first region of the capital fragment and a third anchor pin may extend through a second capital fragment opening onto a second region of the capital fragment. The first region and second region may include a dorsal aspect region of the capital fragment. The second anchoring pin and the third anchoring pin may converge at capital fragment region. The second anchoring pin and third anchoring pin are extended obliquely or at an angled orientation relative to the bone.

A fourth anchoring pin may extend through an opening located on the tarsal base of the first actuation mechanism onto a proximal region of the body of the first metatarsal. A fifth anchoring pin may extend through an opening located on the tarsal base of the first actuation mechanism onto one or more cuneiform bones. The one or more cuneiform bones may include the medial cuneiform bone. The fourth and fifth anchoring pin extends obliquely and/or at an orientation angle relative to the bone. If desired, a sixth anchoring pin may extend through an opening located on the tarsal base of the first actuation mechanism onto a distal region of the body of the first metatarsal. The fourth, fifth and/or sixth anchoring pin extends obliquely and/or at an orientation angle relative to the bone. The fourth, fifth and/or sixth anchoring pins may extend orthogonal relative to the bone. Furthermore, if desired, the first anchoring pin could be removed to allow easier operation of the bunion guide system.

The anchoring pins may comprise K-wires or other pins or wires known in the art. The anchoring pins may be shorter than conventional anchoring pins due to the close contact of the bottom surfaces position relative to the bone surface. The anchoring pin length may comprise 150 mm to 300 mm; length may comprise 150 mm to 250 mm; length may comprise 150 mm to 200 mm; and/or 150 mm to 175 mm. The anchoring pins may comprise threads. The threads may be disposed at one end of the anchoring pins.

As shown in FIG. 65E, the positioning of the first, second, third and fourth one or more anchoring pins into the desired bones and through portions of the bunion guide create or form a stability triangle. The bunion guide should be properly balanced and leveled to eliminate a major incline angles. Incline angles may increase workload and parallel forces. As each of the actuators are used during the deformity correction procedure, higher loads or forces will be seen at the base of the stability triangle, which is d1, d2 and d3. This triangular stability achieved through the distribution of the anchoring pins minimizes the forces that the bunion guide transmits to the anchor pins and surrounding actuators, as well as eliminating any undesired inertia forces, rotation and/or angular momentum caused by any linked actuators of the bunion guide system. The forces observed on the base of the triangle and/or the anchoring pins are evenly distributed providing a force equilibrium, and prevent the surgeon from experiencing excessive forces during operation of the actuators.

In one embodiment, the method of correcting a bunion deformity comprises the step of resecting the head of the first metatarsal or capital fragment. The osteotomy site is confirmed and performed using distal osteotomy techniques. In one exemplary embodiment, the distal osteotomy techniques comprise transverse distal osteotomy techniques. Distal metatarsal osteotomies perform four advantageous basic functions. They can decrease the intermetatarsal angle; realign structural abnormalities in the transverse plane such as abnormal proximal articular set angles; and shorten or maintain the length of the metatarsal. The osteotomy may be performed by a manual or powered osteotomy tool, and/or any tool known in the art to sever bone. The physician should create an incision over the medial aspect of the first metatarsal neck in location of the planned transverse osteotomy just proximal to the level of the sesamoids.

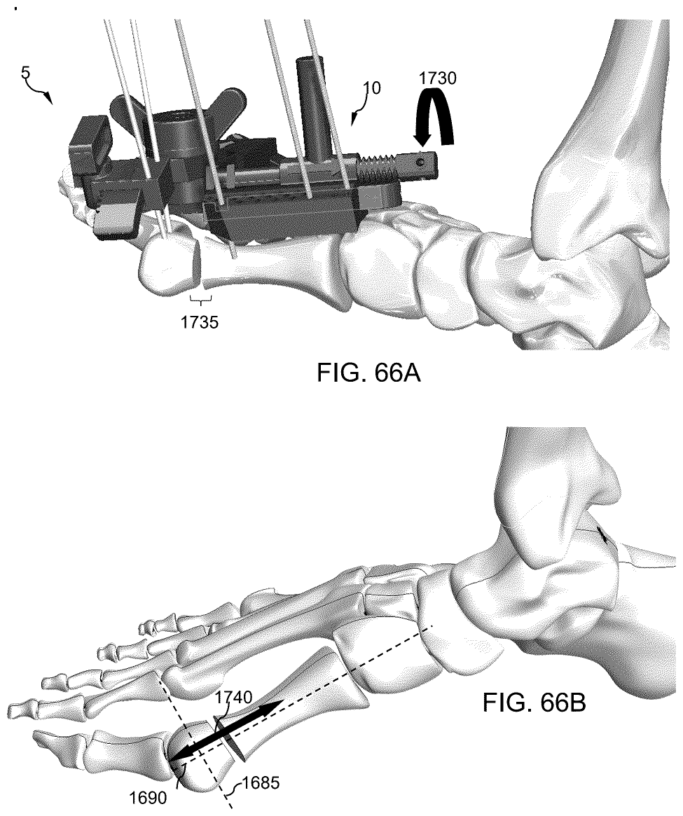

In one embodiment, the method of correcting a bunion deformity comprises the step of distracting the capital fragment or moving the capital fragment in a first direction and a first motion as shown in FIGS. 66A-66B. The first actuation mechanism or the “PINK” or “VIOLET” indicator will be used to distract the capital fragment (and adjoining bones and joints) away from the body of the first metatarsal. The capital fragment continues to be joined to the Great Toe (e.g., the proximal and distal phalanges) through the metaphalangeal joint. The first motion comprises distraction or translation of the body of the first metatarsal towards the posterior direction and/or towards the proximal end of the foot. The distraction or translation occurs along the longitudinal axis of the body of the first metatarsal. The distraction screw of the first actuation mechanism is rotated in the clockwise direction to enable the distraction screw to move, urge, pull relative to the tarsal base the body of the first metatarsal away from the capital fragment at a distraction distance. The distraction distance may comprise 1 to 5 mm; and/or 5 mm or less. The capital fragment distraction distance may be fixed to the desired location. The first direction or first position should be confirmed with imaging. If the desired first position is not reached, adjust using the first actuation mechanism.

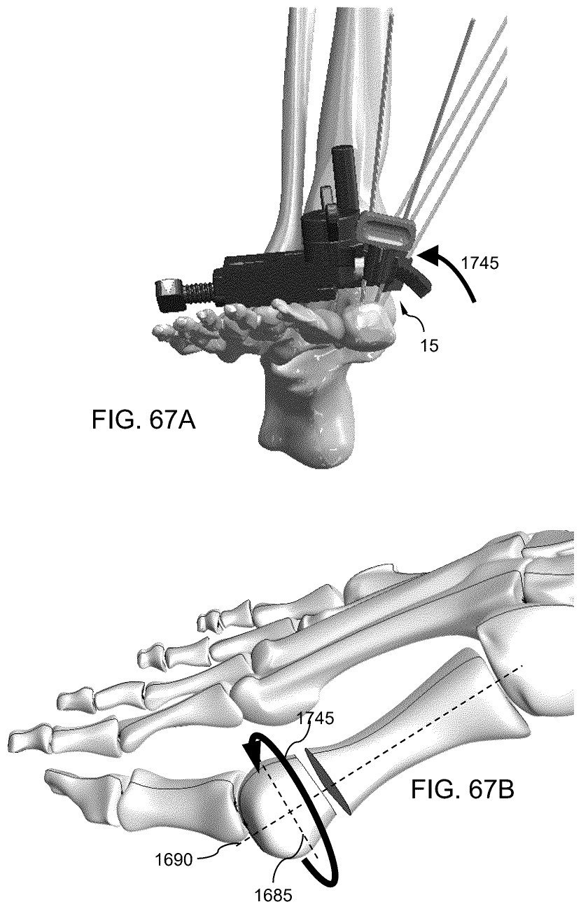

In one embodiment, the method of correcting a bunion deformity comprises the step of rotating the capital fragment or moving the capital fragment in a second direction and a second motion as shown in FIGS. 67A-67B. The second actuation mechanism or the “RED” indicator will be used to rotate the capital fragment while at least two anchoring pins are anchored through the second actuation mechanism to fix the capital fragment. Turning of the rotational gear knob of the second actuation mechanism causes the rotational knob to engage with the teeth of the rotational arc rack and rotate the capital fragment in a desired arc length. The second motion comprises rotation of the capital fragment in a clockwise direction up to an arc length up to 25 degrees and/or 25 degrees or less. The capital fragment will rotate clockwise around or substantially around the longitudinal axis of the body of the first metatarsal. The rotation may facilitate the turning of the sesamoids to face a more natural position or downward position or inferior position. The second direction or second position should be confirmed with imaging. If the desired second position is not reached, adjust using the second actuation mechanism.

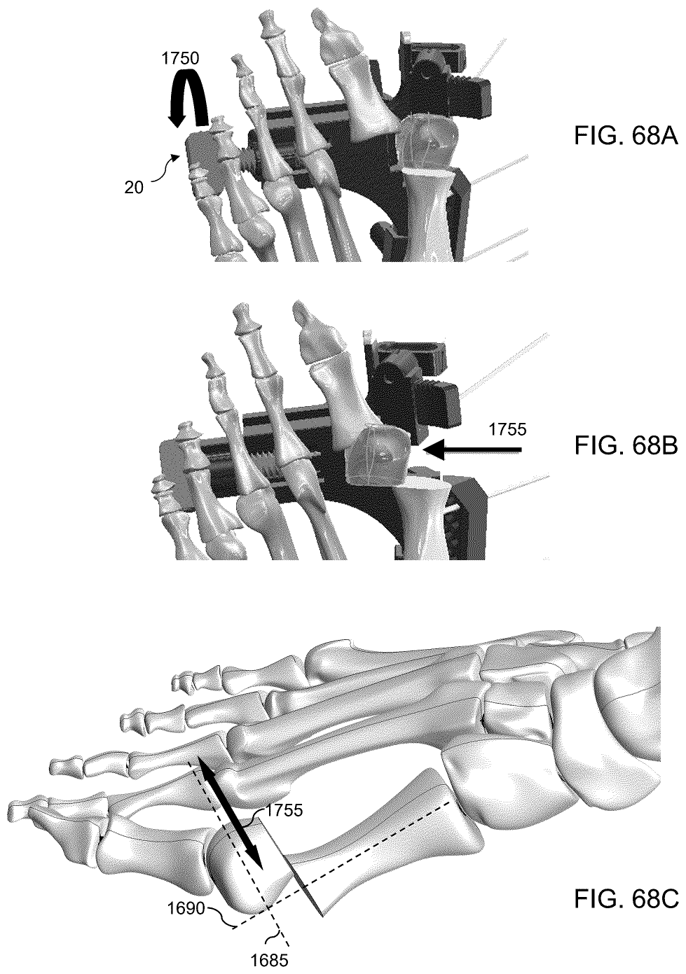

In one embodiment, the method of correcting a bunion deformity comprises the step of translating or displacing the capital fragment or moving the capital fragment in a third direction and a third motion as shown in FIGS. 68A-68C. The third actuation mechanism or the “YELLOW” indicator will be used to move or displace the capital fragment. The third motion comprises displacement or translation of the capital fragment from a lateral direction towards the medial direction if you are viewing in the frontal plane. Alternatively, the third motion comprises displacement or translation of the capital fragment from a medial direction towards the lateral direction. The third motion comprises displacement or translation of capital fragments from the current position of the first metatarsal towards the second metatarsal. The displacement or translation occurs transverse to the longitudinal axis of the body of the first metatarsal and/or follows the second trajectory axis. The displacement or translation screw of the third actuation mechanism is rotated in the clockwise direction to enable the displacement screw to move, urge, pull the capital fragment transversely relative to the tarsal base or the longitudinal axis of the body of the first metatarsal to a desired displacement distance. The translation or displacement distance may comprise 1 to 25 mm; and/or 25 mm or less. The capital fragment displacement distance may be fixed to the desired location. The third direction or third position should be confirmed with imaging. If the desired third position is not reached, adjust using the third actuation mechanism.

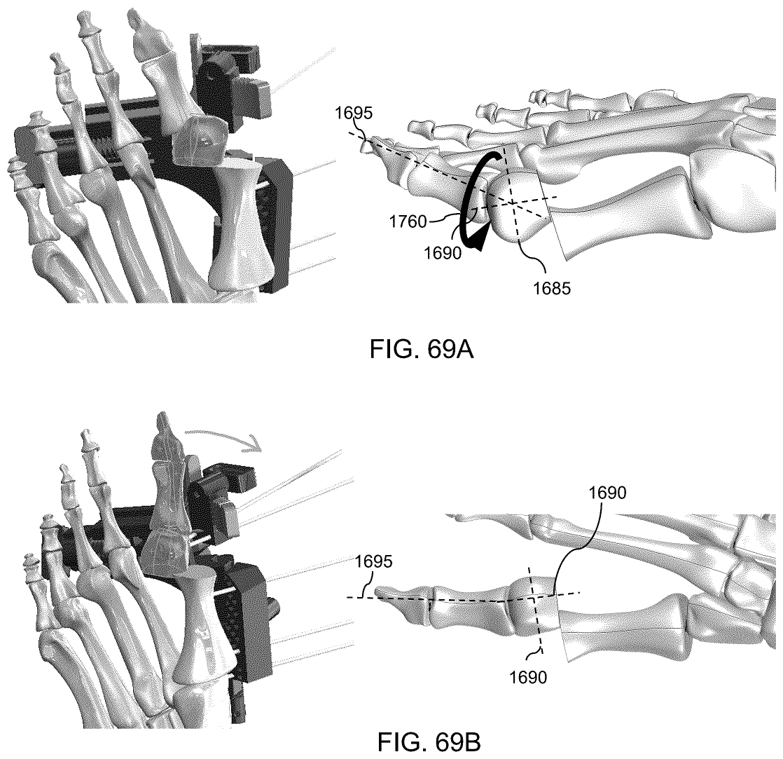

In one embodiment, the method of correcting a bunion deformity comprises the step of abducting and inversion of the capital fragment or moving the capital fragment in a fourth direction and a fourth motion as shown in FIGS. 6A-69B. The fourth actuation mechanism will be used to abduct and invert the capital fragment for final alignment of the Great Toe to correct the PASA or the DMAA. The fourth motion comprises the abduction and inversion of the capital fragment to ensure that the longitudinal axis of the capital fragment aligns or follows with the longitudinal axis of the Great Toe. The rotation of the PASA or DMAA pin allows the capital fragment to rotate to ensure alignment with the great toe and/or the second metatarsal. The adduction and/or inversion of the capital fragment may be up to 15 degrees; and/or 15 degrees or less. The fourth direction or fourth position should be confirmed with imaging. If the desired fourth position is not reached, adjust using the fourth actuation mechanism.

In one embodiment, the method of correcting a bunion deformity comprises the step of stabilizing or fixating the final position of the capital fragment relative to the body of the first metatarsal. The bunion guide system will hold the final positioning (fixed in space relative to desired final position) of the capital fragment, which the final positioning is in a more aligned anatomical trajectory. The fixation guide arm assembly will be used to stabilize or fixate the capital fragment in its final position. The step of stabilizing or fixating the bunion guide assembly includes the steps of: positioning the guide arm assembly onto the bunion guide system; obtaining a first screw trajectory at a first position or location; securing at least a portion of the guide arm assembly at the first location; deploying a first fixation screw at the first location to follow the first trajectory; obtaining a second screw trajectory at a second position or location; securing the at least a portion of the guide arm assembly at the second location; and/or deploying a second fixation screw at the second location to follow the second trajectory.

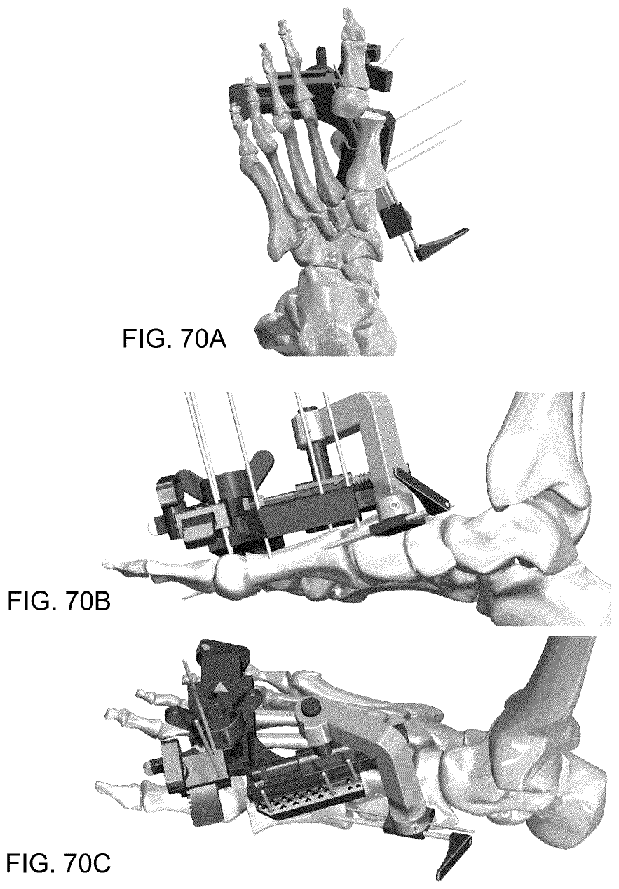

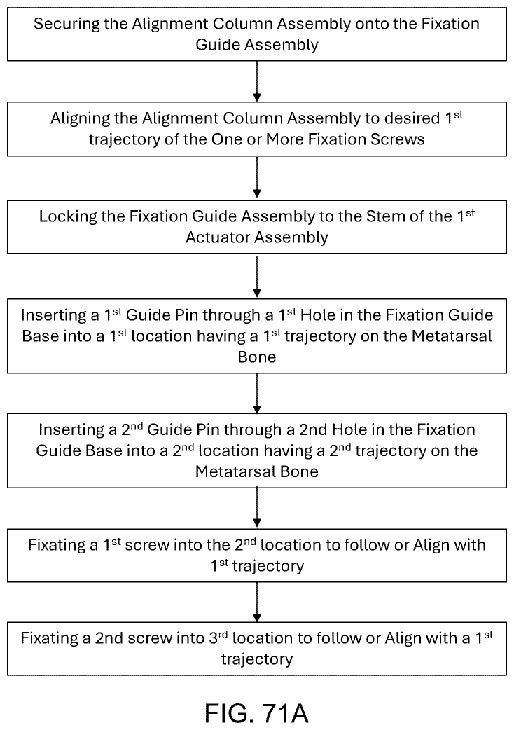

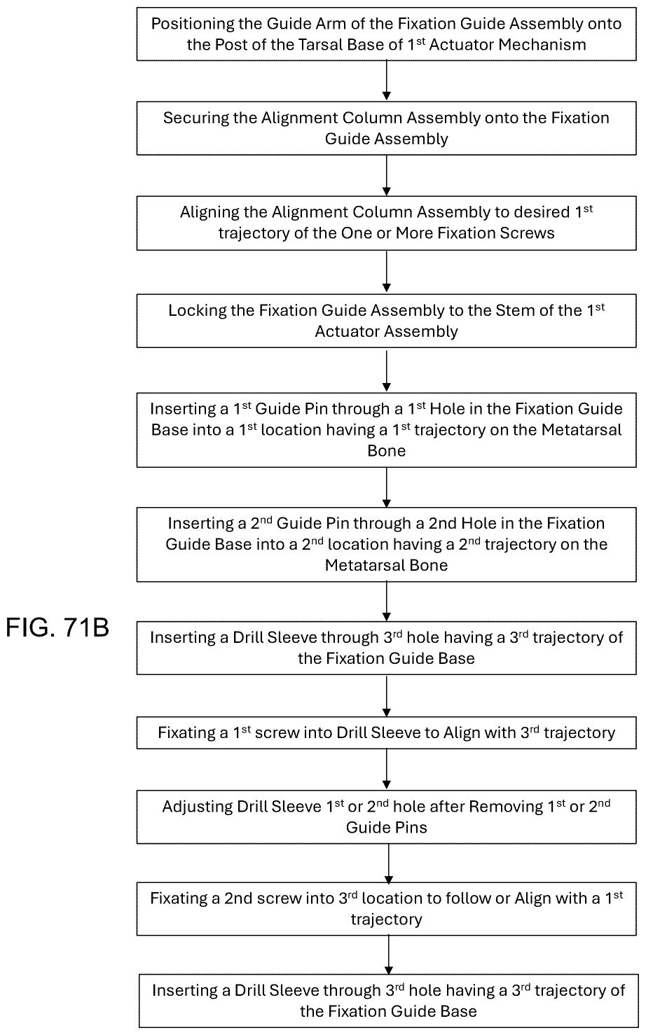

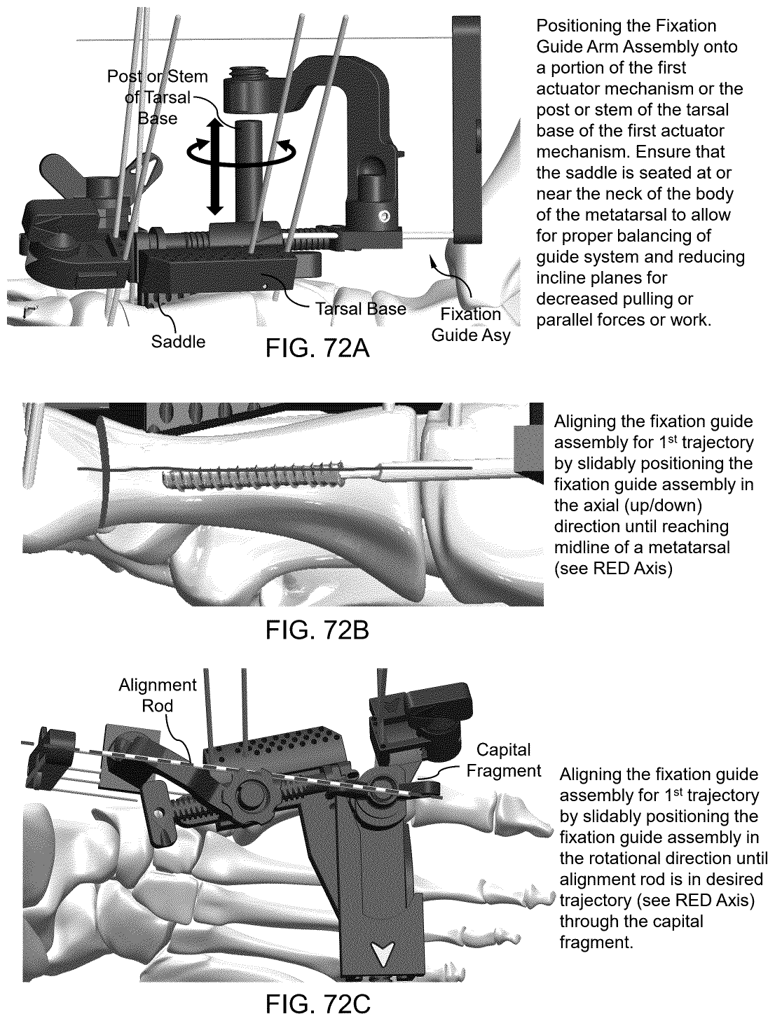

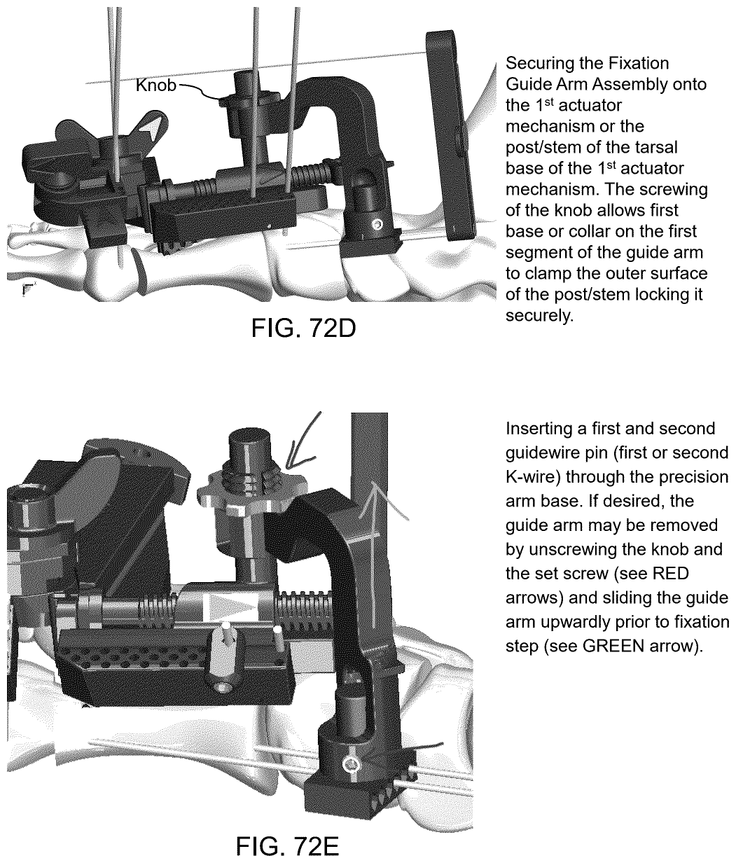

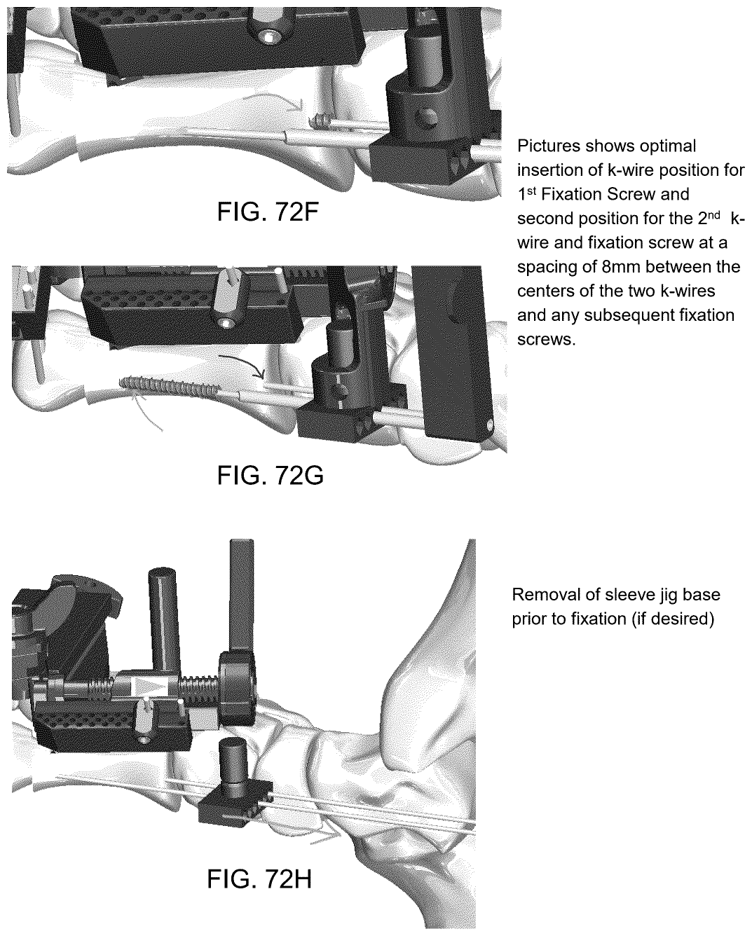

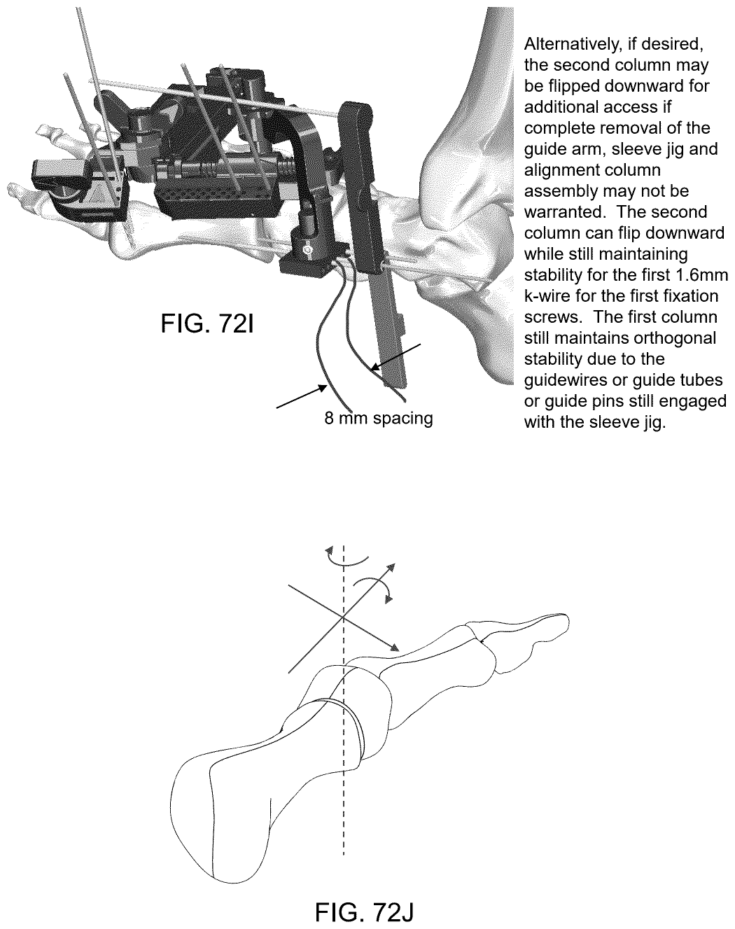

In another embodiment, FIGS. 70A-70C, 71A-71B and 72A-72J illustrates an alternate embodiment of a method of stabilizing or fixating the final position of the capital fragment. The alternate method of stabilizing or fixation the final position of the capital fragment comprises the steps of: positioning the Fixation Guide Assembly onto the tarsal base; aligning the fixation guide assembly to a desired first trajectory of a first fixation screw; securing the fixation guide assembly to the tarsal base; inserting a first guide pin though a first hole in the sleeve jig or sleeve base into a first location on the metatarsal bone; inserting a drill sleeve through a second hole in the sleeve jig or base into a second location on the metatarsal; fixating or securing or fastening a first fixation screw to follow or align with desired first trajectory; adjusting the drill sleeve through a third hole in the sleeve jig or base; and fixating or securing or fastening a second fixation screw into a third location to follow a desired second trajectory, the second trajectory being parallel to the first trajectory.

The positioning step allows the fixation guide arm assembly to be disposed onto a portion of the first actuator mechanism. Accordingly, the positioning step allows the fixation guide arm assembly to be disposed onto the stem or post of the tarsal base of the first actuator mechanism. The alignment step may comprise multi-axial adjustment to achieve the desired trajectory. The multi-axial adjustment includes axial positioning (up and down) and rotational positioning. The axial positioning comprises a travel distance of at least 20 mm or greater or a range of 0 to 40 mm. The rotational positioning comprises a range of 0 to 60 degrees.

The alignment step ensures that the desired trajectory is confirmed. The physician or surgeon should ensure that the first location of the desired first trajectory is aligned to a midline axis of a metatarsal body. The metatarsal body may include a first, second, third, fourth and/or fifth metatarsal. The midline axis may be confirmed through imaging. The physician or surgeon may also ensure that the alignment rod from a coronal view is in the desired first trajectory through a portion of the capital fragment. The post or stem of the tarsal base may include a textured surface, the textured surface provides friction during positioning and alignment step. The friction allows the guide arm to stay in axial and rotational position prior to locking the guide arm in its desired first trajectory. Once the final alignment and positioning is achieved, the guide arm may be locked to the first trajectory position.

The guide arm of the fixation guide assembly includes a first base or collar with a threaded shaft extending from the top surface of the collar or first base. The threaded shaft and/or the collar includes a cylindrical inner surface and a conical outer surface. While the knob is screwed onto the threaded shaft, the threaded shaft and/or the collar can be squeezed against such that its inner surface contracts to a slightly smaller diameter, squeezing or clamping the external surface or outer diameter of the stem/post of the tarsal base to hold it securely or lock it into place.

Inserting a first guidewire (or first K-wire) through a first hole in the precision screw arm base into a first location on the metatarsal bone to match or substantially match the midline metatarsal anatomic axis. The first guide pin allows the alignment column assembly to maintain balance and stability. The first guidewire may be inserted through the drill sleeve and/or sleeve jig of the fixation guide arm assembly to be introduced along the proximal aspect of the body of the first metatarsal to follow or align or substantially follow or align with the intended trajectory position. The intended trajectory position may follow or align with the lateral aspect of the capital fragment. The guidewire trajectory is confirmed with imaging to ensure that it matches or substantially matches the intended trajectory position.

If desired a second guidewire may be inserted through the drill sleeve and/or sleeve jig to enter distal to the proximal or first guidewire in a parallel fashion into a third hole. The second guidewire may be positioned at or near the central aspect of the capital fragment to ensure fixation. Optimal spacing between each fixation screw may comprise 8 mm. However, spacing between each fixation screw may comprise at least 4 mm. In another exemplary embodiment, the physician or surgeon may decide to fixate the first fixation screw into the first location prior to inserting the second guidewire at a second location to fixate a second fixation screw into the second location.

If desired, a third guidewire may be inserted into a third location. The third hole engagement with the third guidewire keeps the alignment rod of the alignment column assembly orthogonal to the sleeve jig Base and subsequently orthogonal to the entire bunion guide system, as well as the intended direction trajectory of the fixation screws through the capital fragment to maintain stability for deformity correction.

After the one or more guidewires are confirmed for proper positioning, the fixation screw lengths should be measured and selected. Screw lengths should be carefully measured to obtain as much length and bony fixation as possible. The selected fully threaded screws are placed over the one or more guidewires (e.g., the first and second guidewires) and across the osteotomy site. The surgeon or physician may elect to remove the guide arm assembly and/or the sleeve jig base after K-wires or guidewires are positioned. Alternatively, the second column of the alignment column assembly may be rotated inferiorly or downwardly for ease of access if warranted. If possible, the proximal fixation screw is placed first prior to the second screw into the capital fragment. The one or more fixation screws may comprise 3.5 mm or 4 mm full threaded, headless non-compression screws with beveled tips to secure the osteotomy site. Other one or more fixation screws known in the art may be used. The bicortical fixation without soft tissue disruption of the vascularized tissues secures the osteotomy and permits bony remodeling laterally.

In one embodiment, the method of correcting a bunion deformity comprises the step of obtaining final images to demonstrate hallux valgus correction. Final A/P and lateral images may be obtained. The A/P images may be used to demonstrate the final hallux valgus correction and secure fixation with restoration of a normal or substantially normal hallux valgus angle (HVA) and sesamoid coverage. The lateral images may be used to demonstrate appropriate screw placement, trajectory and alignment into the central aspect of the capital fragment.

### Bunion Guide Sterilized Kit

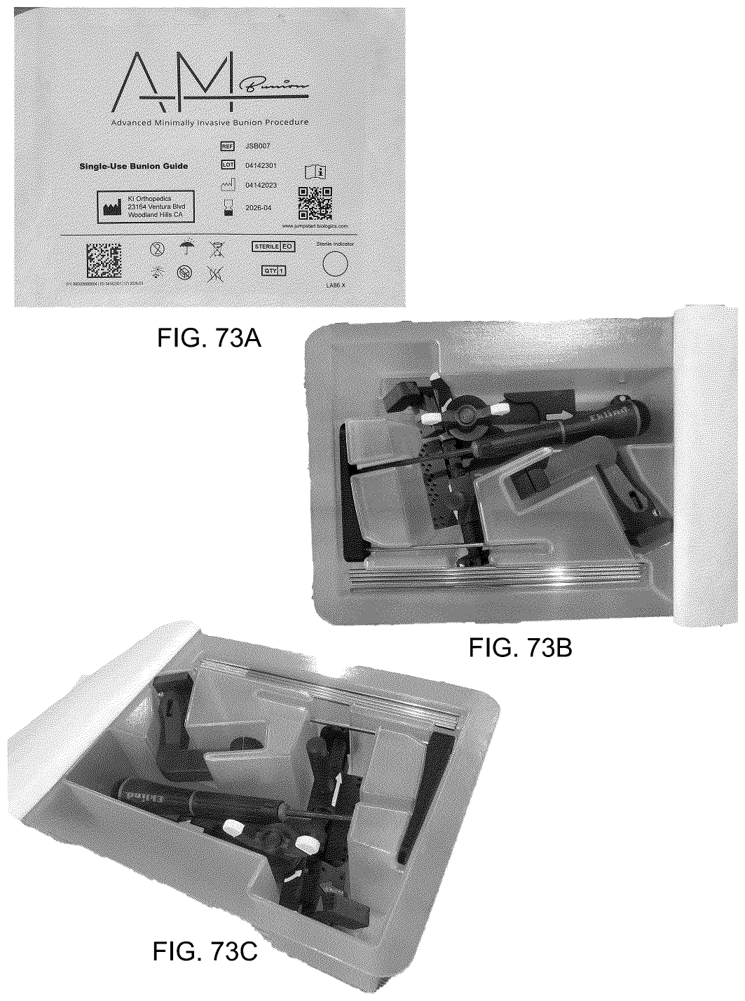

FIGS. 73A-73C illustrate different views of a bunion guide system that comprises a sterile packaged kit. The sterile packaged kit includes at least one bunion guide system assembly, the at least one bunion guide system assembly comprises a bunion guide system and bunion guide instruments. The bunion guide system includes a first actuator, a second actuator, a third actuator, a fourth actuator, a guide arm, and a drill sleeve. The bunion guide instruments include a driver, one or more anchoring pins, one or more fixation screws and a guide system package adapted to receive the at least one bunion guide system and related instruments. The guide system package maintains the at least one bunion guide system and the guide system instruments sterile after sterilization of the sterile packaged kit.

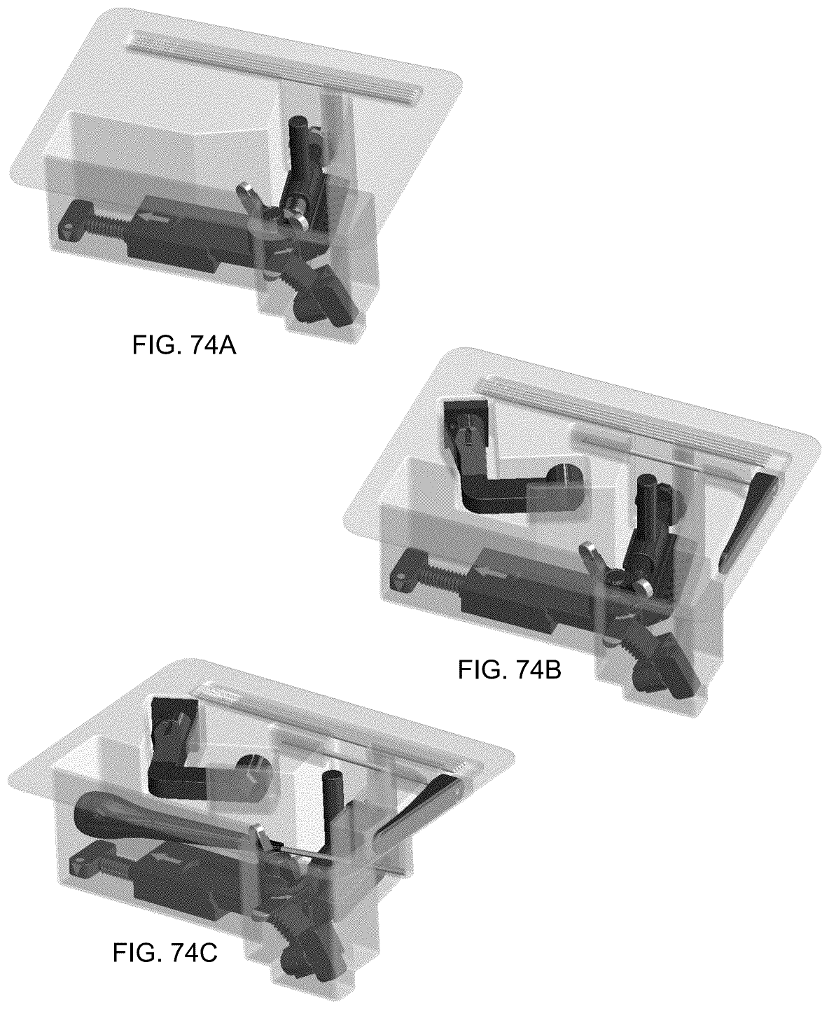

FIGS. 74A-74C illustrate different views of a method of packaging a bunion guide system assembly into a bunion guide system packaging. A method of packaging a bunion guide system assembly comprises the steps of: inserting at least one bunion guide system into a first compartment of the packaging tray; inserting or securing the guide arm and the drill sleeve into a second and third compartment of the packaging tray; securing the one or more anchor pins into a fourth compartment of the packaging tray; inserting the driver into a fifth compartment of the packaging tray. The packaging tray is enclosed by inserting an outer cover or lid over the packaging tray and sealing over the opening and flange of the packaging tray. The enclosed packaging tray and the bunion guide system assembly therein are sterilized. The at least one bunion guide system may be fully assembled and not requiring assembly once surgeon is ready to use the system during the procedure. Alternatively, the at least one bunion guide system may be separate into its modular components within the packaging tray, requiring assembly during the procedure.

I/We claim:

1. A bunion guide system, comprising:
an angled base plate;
a first actuator mechanism, the first actuator mechanism comprises a tarsal base and a distraction screw, at least a portion of the distraction screw being fixed to the angled base plate, at least a portion of the distraction screw extending through a portion of the base, the base being movable relative to the distraction screw;
a second actuator mechanism, the second actuator mechanism comprises an arc rack, a gear knob and a rotational block, at least a portion of the arc rack extends through the rotational block, the gear knob engages with the arc rack to allow the arc rack to be movable relative to the rotation knob;
a third actuator mechanism, the third actuator mechanism comprises a housing and a translation screw, at least a portion of the translation screw being fixed to the angled base plate, at least a portion of the translation screw extending through the housing, the housing being movable relative to the translation screw; and
a fourth actuator mechanism, the fourth actuator mechanism comprises a pin and knob, the pin is inserted through at least one opening on the housing of the third actuator mechanism, the pin engages with a portion of the arc rack of the second actuator mechanism to allow the arc rack to be movable relative to the angled base plate.

2. A method of correcting hallux valgus comprises the steps of:
Aligning a bunion guide system to a first axis and a second axis on a portion of the outer surface of the foot, the foot includes a metatarsal having a capital fragment and a metatarsal body, the metatarsal body including a metatarsal body longitudinal axis;
resecting the capital fragment from the metatarsal body;
Distracting the metatarsal body from the capital fragment in a first direction using a first actuation mechanism of the bunion guide system;
Rotating the capital fragment in a second direction using a second actuation mechanism of the bunion guide system;
Displacing the capital fragment in a third direction using a third actuation mechanism of the bunion guide system;
Rotating the capital fragment in a fourth direction using a fourth actuation mechanism of the bunion guide assembly to obtain a final corrected position; and
stabilizing the capital fragment in the final corrected position.

3. The method of claim 2, wherein the metatarsal can be selected from the group consisting of a first metatarsal, a second metatarsal, a third metatarsal, a fourth metatarsal, a fifth metatarsal, and any combination thereof.

4. The method of claim 2, wherein the portion of the outer surface of the foot comprises a superior outer surface.

5. The method of claim 2, wherein the first axis comprises aligning or being parallel to the metatarsal body longitudinal axis and the second axis comprises aligning substantially transverse to the metatarsal body longitudinal axis of the foot.

6. The method of claim 2, wherein the first axis comprises aligning or being parallel to the metatarsal body longitudinal axis and the second axis comprises aligning or being parallel to the slope of the deep transverse metatarsal ligament.

7. The method of claim 5, wherein the substantially transverse comprises less than 90 degrees and greater than 75 degrees.

8. The method of claim 2, wherein the method further comprises anchoring the aligned bunion guide system to the foot.

9. The method of claim 2, wherein the first direction is a translation along the metatarsal body longitudinal axis towards the posterior direction of the foot.

10. The method of claim 2, wherein the second direction is a rotation around the metatarsal body longitudinal axis towards the lateral direction or clockwise direction.

11. The method of claim 2, wherein the third direction is a translation substantially transverse to the metatarsal body longitudinal axis towards the lateral direction.
The method of claim 2, wherein the fourth direction is a rotation around the central vertical axis.

12. The method of claim 8, wherein the anchoring step comprises the step of:
Extending a first anchoring pin through a first portion of the bunion guide system and into first location on the metatarsal;
Extending a second anchoring pin through a second portion of the bunion guide system and into a second location on the metatarsal;
Extending a third anchoring pin through a third portion of the bunion guide system and into a third location on the metatarsal; and
Extending a fourth anchoring pin through a fourth portion of the bunion guide system and into a portion of the midfoot bones or tarsal bones.

13. The method of claim 12, wherein the midfoot bones or tarsal bones comprise cuneiform bones, navicular bone, cuboid bone and/or any combination thereof.

14. The method of claim 12, wherein the first location comprises a dorsal location on the capital fragment of the metatarsal.

15. The method of claim 12, wherein the second location comprises a distal portion of the metatarsal body that aligns on the metatarsal body longitudinal axis.

16. The method of claim 12, wherein the second location comprises a distal portion of the metatarsal body that is adjacent or parallel to the metatarsal body longitudinal axis.

17. The method of claim 12, wherein the third location comprises a proximal portion of the metatarsal body that is adjacent or parallel to the metatarsal body longitudinal axis.

18. The method of claim 2, wherein stabilizing the capital fragment comprises the steps of:
Aligning the fixation guide arm assembly to desired first trajectory of first fixation screw;
Locking the fixation guide arm assembly onto a portion of the first actuator mechanism;
Inserting a first guidewire through a first hole in the sleeve base of the fixation guide assembly into a first location into the metatarsal body;
Inserting a second guidewire through a second hole in the sleeve base of the fixation guide assembly into a second location into the metatarsal body, the second hole is spaced apart from the first hole at a set distance;
Fixating a first fixation screw into the first location to the desired first trajectory; and
Fixating a second fixation screw into the second location to be parallel to the desired first trajectory.

19. The method of claim 18, wherein the set distance comprises 4 mm or 8 mm.

20. The bunion guide system of 1, wherein the first motion comprises translation, the second motion comprises rotation, the third motion comprises translation and the fourth motion comprises rotation.
